SEQUENCE LISTING (74 sequences)

SEQ ID NO: 1 - DNA, 21 bp, Artificial Sequence
  Synthetic
gagcacaaauaauuguuuutt

SEQ ID NO: 2 - DNA, 21 bp, Artificial Sequence
  Synthetic
aaaacaauuauuugugcucta

SEQ ID NO: 3 - DNA, 58 bp, Homo sapiens
ccgggccatgggtgtgacactatacctcgaggtatagtgtcacacccatggctttttg

SEQ ID NO: 4 - DNA, 59 bp, Homo sapiens
ccggcccgatgcttctgtttcattcctcgaggaatgaaacagaagcatcgggttttttg

SEQ ID NO: 5 - DNA, 59 bp, Homo sapiens
ccgggcaaagaggacgcccataattctcgagaattatgggcgtcctctttgcttttttg

SEQ ID NO: 6 - DNA, 59 bp, Homo sapiens
ccggcctctcatccttgagcatccactcgagtggatgctcaaggatgagaggttttttg

SEQ ID NO: 7 - DNA, 58 bp, Homo sapiens
ccggatcgtcatctctggttatttgctcgagcaaataaccagagatgacgattttttg

SEQ ID NO: 8 - DNA, 58 bp, Homo sapiens
ccggtcgtcaagttggcctacaatgctcgagcattgtaggccaacttgacgatttttg

SEQ ID NO: 9 - DNA, 59 bp, Homo sapiens
ccggcaatacctactatgcaatgaactcgagttcattgcatagtaggtattgttttttg

SEQ ID NO: 10 - DNA, 57 bp, Homo sapiens
ccggcgagcggatcatgtgtttacactcgagtgtaaacacatgatccgctcgttttt

SEQ ID NO: 11 - DNA, 57 bp, Homo sapiens
ccggccgtttctacttccaggatctctcgagagatcctggaagtagaaacggttttt

SEQ ID NO: 12 - DNA, 57 bp, Homo sapiens
ccgggtgaagaccatgatacgtaaactcgagtttacgtatcatggtcttcacttttt

SEQ ID NO: 13 - DNA, 57 bp, Homo sapiens
ccggcgaccctttctactatgcattctcgagaatgcatagtagaaagggtcgttttt

SEQ ID NO: 14 - DNA, 57 bp, Homo sapiens
ccggagtcaaacacattcccagcttctcgagaagctgggaatgtgtttgactttttt

SEQ ID NO: 15 - DNA, 58 bp, Mus sp.
ccggccatgattcgaaagcgctcatctcgagatgagcgctttcgaatcatggtttttg

SEQ ID NO: 16 - DNA, 58 bp, Mus sp.
ccgggtatccacttgggcatggaatctcgagattccatgcccaagtggatactttttg

SEQ ID NO: 17 - DNA, 58 bp, Mus sp.
ccggtttcccgaccagcccgatatactcgagtatatcgggctggtcgggaaatttttg

SEQ ID NO: 18 - DNA, 57 bp, Mus sp.
ccggcggtgtaagcaacgagttcaactcgagttgaactcgttgcttacaccgttttt

SEQ ID NO: 19 - DNA, 57 bp, Mus sp.
ccgggctgaatcagtacaccctgaactcgagttcagggtgtactgattcagcttttt

SEQ ID NO: 20 - DNA, 57 bp, Mus sp.
ccggccatgattcgaaagcgctcatctcgagatgagcgctttcgaatcatggttttt

SEQ ID NO: 21 - DNA, 57 bp, Mus sp.
ccgggtatccacttgggcatggaatctcgagattccatgcccaagtggatacttttt

SEQ ID NO: 22 - DNA, 57 bp, Mus sp.
ccggccctttcatggatgaacgaatctcgagattcgttcatccatgaaagggttttt

SEQ ID NO: 23 - DNA, 58 bp, Mus sp.
ccgggactccatgtcgtcgactttgctcgagcaaagtcgacgacatggagtctttttg

SEQ ID NO: 24 - DNA, 58 bp, Mus sp.
ccggcggtgtaagcaacgagttcaactcgagttgaactcgttgcttacaccgtttttg

SEQ ID NO: 25 - DNA, 58 bp, Mus sp.
ccggttgttggatttccgtagtattctcgagaatactacggaaatccaacaatttttg

SEQ ID NO: 26 - DNA, 58 bp, Mus sp.
ccgggaatcctcatagaccttattactcgagtaataaggtctatgaggattctttttg

SEQ ID NO: 27 - DNA, 58 bp, Mus sp.
ccgggaccataaatttaccataaagctcgagctttatggtaaatttatggtctttttg

SEQ ID NO: 28 - DNA, 58 bp, Mus sp.
ccggcacgagttgaccggacataaactcgagtttatgtccggtcaactcgtgtttttg

SEQ ID NO: 29 - DNA, 57 bp, Mus sp.
ccggcccatcttatagttcaaccatctcgagatggttgaactataagatgggttttt

SEQ ID NO: 30 - DNA, 57 bp, Mus sp.
ccgggcaatcaagcagttggattatctcgagataatccaactgcttgattgcttttt

SEQ ID NO: 31 - DNA, 57 bp, Mus sp.
ccggcctgaatcgaaatgtgttcttctcgagaagaacacatttcgattcaggttttt

SEQ ID NO: 32 - DNA, 57 bp, Mus sp.
ccggcgtagtattgatgatgagattctcgagaatctcatcatcaatactacgttttt

SEQ ID NO: 33 - DNA, 57 bp, Mus sp.
ccggccacagaaatccaaacaccaactcgagttggtgtttggatttctgtggttttt

SEQ ID NO: 34 - DNA, 59 bp, Mus sp.
ccgggtagctgtgaagatactcaatctcgagattgagtatcttcacagctacttttttg

SEQ ID NO: 35 - DNA, 58 bp, Mus sp.
ccgggaaggttgtaaacccatattactcgagtaatatgggtttacaaccttctttttg

SEQ ID NO: 36 - DNA, 59 bp, Mus sp.
ccgggtgacctcacttgactcttctctcgagagaagagtcaagtgaggtcacttttttg

SEQ ID NO: 37 - DNA, 59 bp, Mus sp.
ccgggcacagacaattgcagtaaatctcgagatttactgcaattgtctgtgcttttttg

SEQ ID NO: 38 - DNA, 57 bp, Mus sp.
ccgggcataataagtcacagccaaactcgagtttggctgtgacttattatgcttttt

SEQ ID NO: 39 - DNA, 57 bp, Mus sp.
ccggccatcctgaaagagtaccattctcgagaatggtactctttcaggatggttttt

SEQ ID NO: 40 - DNA, 57 bp, Mus sp.
ccggtgattgatgatgaagccttaactcgagttaaggcttcatcatcaatcattttt

SEQ ID NO: 41 - DNA, 57 bp, Mus sp.
ccggcctggaagtcacacaatagaactcgagttctattgtgtgacttccaggttttt

SEQ ID NO: 42 - DNA, 57 bp, Mus sp.
ccgggttgcctaccatctcataatactcgagtattatgagatggtaggcaacttttt

SEQ ID NO: 43 - DNA, 58 bp, Mus sp.
ccggctactggatttccgtagtattctcgagaatactacggaaatccagtagtttttg

SEQ ID NO: 44 - DNA, 59 bp, Homo sapiens; identical to SEQ ID NO: 34

SEQ ID NO: 45 - DNA, 58 bp, Homo sapiens; identical to SEQ ID NO: 35

SEQ ID NO: 46 - DNA, 59 bp, Homo sapiens; identical to SEQ ID NO: 36

SEQ ID NO: 47 - DNA, 59 bp, Homo sapiens; identical to SEQ ID NO: 37

SEQ ID NO: 48 - DNA, 57 bp, Homo sapiens; identical to SEQ ID NO: 38

SEQ ID NO: 49 - DNA, 57 bp, Homo sapiens; identical to SEQ ID NO: 39

SEQ ID NO: 50 - DNA, 57 bp, Homo sapiens; identical to SEQ ID NO: 40

SEQ ID NO: 51 - DNA, 57 bp, Homo sapiens; identical to SEQ ID NO: 41

SEQ ID NO: 52 - DNA, 57 bp, Homo sapiens; identical to SEQ ID NO: 42

SEQ ID NO: 53 - DNA, 58 bp, Homo sapiens; identical to SEQ ID NO: 43

SEQ ID NO: 54 - DNA, 57 bp, Mus sp.
ccgggcccagatgaacgctaagatactcgagtatcttagcgttcatctgggcttttt

SEQ ID NO: 55 - DNA, 57 bp, Mus sp.
ccggcaggccataaagtggcagttactcgagtaactgccactttatggcctgttttt

SEQ ID NO: 56 - DNA, 57 bp, Mus sp.
ccggcccaagtctctagctgtgaaactcgagtttcacagctagagacttgggttttt

SEQ ID NO: 57 - DNA, 57 bp, Mus sp.
ccggcgccagtcttatcactgctttctcgagaaagcagtgataagactggcgttttt

SEQ ID NO: 58 - DNA, 57 bp, Mus sp.
ccggcgagcgactatcaaagacatactcgagtatgtctttgatagtcgctcgttttt

SEQ ID NO: 59 - DNA, 58 bp, Mus sp.
ccggtattatctcactcggtattacctcgaggtaataccgagtgagataatatttttg

SEQ ID NO: 60 - DNA, 58 bp, Mus sp.
ccggaccaattgacaggccataaagctcgagctttatggcctgtcaattggttttttg

SEQ ID NO: 61 - DNA, 58 bp, Mus sp.
ccggatttgcccagctacctatttcctcgaggaaataggtagctgggcaaattttttg

SEQ ID NO: 62 - DNA, 58 bp, Mus sp.
ccggctagctgtggatcgccaaattctcgagaatttggcgatccacagctagtttttg

SEQ ID NO: 63 - DNA, 57 bp, Homo sapiens
ccggcgcagtttagatgttgttggactcgagtccaacaacatctaaactgcgttttt

SEQ ID NO: 64 - DNA, 57 bp, Homo sapiens
ccgggtggcttatcatcttatcattctcgagaatgataagatgataagccacttttt

SEQ ID NO: 65 - DNA, 57 bp, Homo sapiens
ccggcccactgaaacgagcaactatctcgagatagttgctcgtttcagtgggttttt

SEQ ID NO: 66 - DNA, 58 bp, Homo sapiens
ccggggtgtaggtaaatctagtttactcgagtaaactagatttacctacacctttttg

SEQ ID NO: 67 - DNA, 57 bp, Homo sapiens
ccgggctgtgtttatcgcccaatttctcgagaaattgggcgataaacacagcttttt

SEQ ID NO: 68 - DNA, 57 bp, Homo sapiens
ccgggctgtgaaagaagtgtgtgaactcgagttcacacacttctttcacagcttttt

SEQ ID NO: 69 - DNA, 59 bp, Homo sapiens
ccggcaactttacctggttgataacctcgaggttatcaaccaggtaaagttgttttttg

SEQ ID NO: 70 - DNA, 59 bp, Homo sapiens
ccgggtcatcctcatattatcaaacctcgaggtttgataatatgaggatgacttttttg

SEQ ID NO: 71 - DNA, 58 bp, Homo sapiens
ccggagatagccgatttcggattatctcgagataatccgaaatcggctatcttttttg

SEQ ID NO: 72 - DNA, 58 bp, Homo sapiens
ccgggagcatgtacctacgttatttctcgagaaataacgtaggtacatgctctttttg

SEQ ID NO: 73 - protein, 15 aa, Artificial Sequence
  Glycine linker
GGGGGGGGGGGGGGG

SEQ ID NO: 74 - protein, 15 aa, Artificial Sequence
  Glycine Serine Linker
GGGGSGGGGSGGGGS

(19) United States
(12) Patent Application Publication
(10) Pub. No.: US 2023/0022117 A1
(43) Pub. Date: Jan. 26, 2023

(54) RNAi-BASED TARGETING COMPOUNDS AND USES THEREOF TO PREVENT ACQUIRED HEARING LOSS

(71) Applicant: MUSC FOUNDATION FOR RESEARCH DEVELOPMENT, CHARLESTON, SC (US)

(72) Inventor: Suhua SHA, Mt. Pleasant, SC (US)

(21) Appl. No.: 17/782,611

(22) PCT Filed: Dec. 4, 2020
(86) PCT No.: PCT/US2020/063478

§ 371 Date: Jun. 3, 2022

Related U.S. Application Data
(60) Provisional application No. 62/943,691, filed on Dec. 4, 2019.
(60) Provisional application No. 62/951,944, filed on Dec. 20, 2019.

(51) Int. Cl. C12N 15/113; A61P 27/16

(52) U.S. Cl. CPC C12N 15/1137; A61P 27/16

(57) ABSTRACT

Described herein are compositions capable of targeting CaMKKβ and/or AMPK alpha and formulations thereof. Also described herein are methods of using the compositions and formulations thereof. In some embodiments, the compositions capable of targeting CaMKKβ and/or AMPK alpha and formulations thereof can prevent or treat outer hair cell loss, such as that which can occur as a result of auditory or chemical insult. In some embodiments, the compositions capable of targeting CaMKKβ and/or AMPK alpha and formulations thereof can treat or prevent acquired hearing loss.

### CROSS-REFERENCE TO RELATED APPLICATIONS

This application claims the benefit of and priority to U.S. Provisional Patent Application No. 62/943,961, filed on Dec. 4, 2019, entitled “CAMKK BETA TARGETING COMPOUNDS, FORMULATIONS, AND USES THEREOF,” and U.S. Provisional Patent Application No. 62/951,944, filed on Dec. 20, 2019, entitled “AMPK TARGETING COMPOUNDS, FORMULATIONS, AND USES THEREOF,” the contents of which are incorporated by reference herein in their entirety.

### STATEMENT REGARDING FEDERALLY SPONSORED RESEARCH

This invention was made with government support under Grant No.(s) DC009222 granted by the National Institutes of Health. The government has certain rights in the invention.

### SEQUENCE LISTING

This application contains a sequence listing filed in electronic form as an ASCII.txt file entitled MUSC-0100WP_ST25.txt, created on Nov. 30, 2020 and has the size of 11,000 bytes. The content of the sequence listing is incorporated herein in its entirety.

### TECHNICAL FIELD

The subject matter disclosed herein is generally directed to therapies for hearing loss.

### BACKGROUND

Acquired hearing loss is caused by numerous etiologies accumulated over a lifetime, such as exposure to excessive noise, the use of ototoxic medications including aminoglycoside antibiotics and the anti-cancer drug cisplatin, bacterial or viral ear infections, head injuries, and the aging process. The common pathology of acquired hearing loss is loss of cochlear sensory hair cells with outer hair cells (OHCs) being more sensitive than inner hair cells (IHCs). This common pathology has been well documented both in humans and in animal models. Since cochlear sensory hair cells in mammals do not regenerate, the loss is irreversible and causes permanent hearing loss. Currently, there are no clinical established pharmaceutical therapies for prevention and treatment of acquired hearing loss.

As such, there exists a need for compositions, techniques, and other technologies for prevention and treatment of hearing loss.

Citation or identification of any document in this application is not an admission that such a document is available as prior art to the present invention.

### SUMMARY

Described in several embodiments herein are methods of treating or preventing an acquired (i) hearing loss, (ii) ototoxicity, (iii) outer hair cell damage or death, (iv) loss of an outer hair cell function, or a combination thereof comprising:
a. administering an amount of an AMPK alpha targeting RNAi molecule to the subject in need thereof,
b. administering an amount of a CaMKKβ targeting RNAi molecule to the subject in need thereof, or
c. a combination thereof.

In certain example embodiments, the AMPK alpha targeting RNAi molecule is capable of specifically binding a target sequence in an AMPK alpha gene product, the CaMKKβ targeting RNAi molecule is capable of specifically binding a CaMKKβ gene product, or both.

In certain example embodiments, the target sequence in the CaMKKβ gene product
a. is 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

In certain example embodiments, the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

In certain example embodiments, the AMPK alpha gene product is an mRNA, wherein the CaMKKβ gene product is an mRNA, or both.

In certain example embodiments, the AMPK alpha gene product is an AMPK alpha isoform 1 encoding polynucleotide or an AMPK alpha isoform 2 encoding polynucleotide.

In certain example embodiments, the AMPK alpha targeting RNAi molecule is a polynucleotide having a sequence that
a. is 50-100 percent identical to a sequence selected from the group consisting of SEQ ID NOs: 25-72; or
b. is 50-100 percent identical to a sequence that is complementary to a sequence selected from the group consisting of SEQ ID NOs: 25-72.

In certain example embodiments, administering the AMPK alpha targeting RNAi molecule, the CaMKKβ targeting RNAi molecule, or both occurs
a. prior to exposure of the subject to an ototoxic agent;
b. after exposure of the subject to an ototoxic agent; or
c. both.

In certain example embodiments, the ototoxic agent is a selected from the group consisting of: an antibiotic, a chemotherapeutic agent, a diuretic, a non-steroidal anti-inflammatory agent, an anti-malarial agent, an industrial solvent, an anticonvulsant agent, a psychopharmacologic agent, a cardiac or blood pressure therapeutic agent, an anti-allergy agent, a quinine-based agent, a glucocorticosteroid, an anesthetic, or a combination thereof.

In certain example embodiments, the ototoxic agent is an aminoglycoside antibiotic.

In certain example embodiments, the ototoxic agent is gentamicin, neomycin, kanamycin, amikacin, streptomycin, tobramycin, netilmicin, vancomycin, erythromycin, or a combination thereof.

In certain example embodiments, the ototoxic agent is a platinum-based chemotherapeutic agent.

In certain example embodiments, the ototoxic agent is cisplatin, carboplatin, oxaliplatin, nitrogen mustard, methotrexate, vincristine, dactinomycin, bleomycin, or any combination thereof.

In certain example embodiments, the diuretic is furosemide, bumetanide, ethacrynic acid, torsemide, chlor-thalidone, or any combination thereof.

In certain example embodiments, the non-steroidal anti-inflammatory agent is an acetic acid-based NSAID, a COX-2 inhibitor, a fenamate, an oxicam, a propionic acid, a salicylate, or a miscellaneous NSAID, or any combination thereof.

In certain example embodiments, the quinine-based agent is chloroquine phosphate, auinacrine hydrochloride, quinine sulfate, or any combination thereof.

In certain example embodiments, the anti-malarial agent is chloroquine and hydroxychloroquine.

In certain example embodiments, the cardiac or blood pressure therapeutic agent is celiprolol, flecaninide, lidocaine, metoprolol, procainamide, propranolol, or quinidine.

In certain example embodiments, the industrial solvent is cyclohexane, dichloromethane, hexane, indane, methyl-chloride, methyl-n-butyl-ketone, precholor-ethylene, styrene, tetrachlor-ethane, toluol, tricholorethylene, or any combination thereof.

In certain example embodiments, the anesthetic is bupivacaine, tetracaine, lidocaine, or a combination thereof.

In certain example embodiments, the glucocorticosteroid is prednisone, prednisolone, adrenocorticotrophic hormone, or any combination thereof.

In certain example embodiments, the psychopharmacologic agent is amitriptyline, a benzodiazepine, bupropion, carbamazepine, diclofensine, doxepin, desipramine, fluoxetine, imipramine, lithium, melitracen, molindone, paroxetine, phenelzine, protriptyline, trazodone, zimeldine, or any combination thereof.

In certain example embodiments, the chemical ototoxic agent is alcohol, arsenic or arsenic-based compound, caffeine, lead, marijuana, nicotine, mercury, or auranofin, or any combination thereof.

In certain example embodiments, the AMPK alpha targeting RNAi molecule, the CaMKKβ targeting RNAi molecule, or both is/are administered directly to an ear of a subject.

In certain example embodiments, the AMPK alpha targeting RNAi molecule, the CaMKKβ targeting RNAi molecule, or both is/are administered via intra-tympanic delivery, intracochlear delivery, semicircular canal delivery, or a combination thereof.

In certain example embodiments, the AMPK alpha targeting RNAi molecule, the CaMKKβ targeting RNAi molecule, or both is/are administered via intra-tympanic delivery on the round window membrane.

In certain example embodiments, the AMPK alpha targeting RNAi molecule, the CaMKKβ targeting RNAi molecule, or both is/are administered via posterior semicircular canal delivery.

Described in certain example embodiments are methods of reducing an amount of an AMPK alpha gene product, a CaMKKβ gene product, or both in a subject in need thereof, the method comprising:
a. administering an amount of an AMPK alpha targeting RNAi molecule to the subject in need thereof,
b. administering an amount of a CaMKKβ targeting RNAi molecule to the subject in need thereof, or
c. a combination thereof.

In certain example embodiments herein, the AMPK alpha targeting RNAi molecule is capable of specifically binding a target sequence in an AMPK alpha gene product, the CaMKKβ targeting RNAi molecule is capable of specifically binding a CaMKKβ gene product, or both.

In certain example embodiments, target sequence in the CaMKKβ gene product is
a. 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

In certain example embodiments, the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

In certain example embodiments, the AMPK alpha gene product is an mRNA, wherein the CaMKKβ gene product is an mRNA, or both.

In certain example embodiments, the AMPK alpha gene product is an AMPK alpha isoform 1 encoding polynucleotide or an AMPK alpha isoform 2 encoding polynucleotide.

In certain example embodiments, the AMPK alpha targeting RNAi molecule is a polynucleotide having a sequence that
a. is 50-100 percent identical to a sequence selected from the group consisting of SEQ ID NOs: 25-72; or
b. is 50-100 percent identical to a sequence that is complementary to a sequence selected from the group consisting of SEQ ID NOs: 25-72.

In certain example embodiments, the AMPK alpha targeting RNAi molecule, the CaMKKβ targeting RNAi molecule, or both is/are administered directly to an ear of a subject.

In certain example embodiments, the AMPK alpha targeting RNAi molecule, the CaMKKβ targeting RNAi molecule, or both is/are administered via intra-tympanic delivery, intracochlear delivery, semicircular canal delivery, or a combination thereof.

In certain example embodiments, the AMPK alpha targeting RNAi molecule, the CaMKKβ targeting RNAi molecule, or both is/are administered via intra-tympanic delivery on the round window membrane.

In certain example embodiments, the AMPK alpha targeting RNAi molecule, the CaMKKβ targeting RNAi molecule, or both is/are administered via posterior semicircular canal delivery.

Described in certain example embodiments herein are pharmaceutical formulations capable of treating or preventing an acquired (i) hearing loss, (ii) ototoxicity, (iii) outer hair cell damage or death, (iv) loss of an outer hair cell function, or a combination thereof comprising:
a. therapeutically effective amount of
d. an AMPK alpha targeting RNAi molecule;
e. a CaMKKβ targeting RNAi molecule; or
f. both; and

a pharmaceutically acceptable carrier.

In certain example embodiments, the AMPK alpha targeting RNAi molecule is capable of specifically binding a target sequence in an AMPK alpha gene product, the CaMKKβ targeting RNAi molecule is capable of specifically binding a CaMKKβ gene product, or both.

In certain example embodiments, the target sequence in the CaMKKβ gene product
a. is 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

In certain example embodiments, the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

In certain example embodiments, the AMPK alpha gene product is an mRNA, wherein the CaMKKβ gene product is an mRNA, or both.

In certain example embodiments, the AMPK alpha gene product is an AMPK alpha isoform 1 encoding polynucleotide or an AMPK alpha isoform 2 encoding polynucleotide.

In certain example embodiments, the AMPK alpha targeting RNAi molecule is a polynucleotide having a sequence that
a. is 50-100 percent identical to a sequence selected from the group consisting of SEQ ID NOs: 25-72; or is
b. 50-100 percent identical to a sequence that is complementary to a sequence selected from the group consisting of SEQ ID NOs: 25-72.

In certain example embodiments, wherein the AMPK alpha targeting RNAi molecule, the CaMKKβ targeting RNAi molecule, or both are contained in one or more vectors of a vector system.

In certain example embodiments, AMPK alpha targeting RNAi molecule, the CaMKKβ targeting RNAi molecule, or both are effective to decrease or eliminate
a. the amount of an AMPK alpha gene product;
b. the amount of an CaMKKβ gene product;
c. gene expression, protein expression, or both of AMPK alpha isoform 1, AMPK alpha isoform 2, or both;
d. gene expression, protein expression, or both of CaMKKβ; or
e. a combination thereof.

In certain example embodiments, the pharmaceutical formulation is adapted for direct delivery to an ear of the subject.

In certain example embodiments, the pharmaceutical formulation is adapted for intra-tympanic delivery, intracochlear delivery, semicircular canal delivery, or a combination thereof.

In certain example embodiments, the pharmaceutical formulation is adapted for intra-tympanic delivery on the round window membrane, posterior semicircular canal delivery, or both.

Described in several embodiments herein are kits composed of

an AMPK alpha targeting RNAi molecule or a pharmaceutical formulation thereof, a CaMKKβ targeting RNAi molecule or a pharmaceutical formulation thereof, or both; and

instructions fixed in a tangible medium of expression, wherein the instructions provide direction to administer the AMPK alpha targeting RNAi molecule or a pharmaceutical formulation thereof, the CaMKKβ targeting RNAi molecule or a pharmaceutical formulation thereof, or both to a subject in need thereof to
a. treat or prevent an acquired
   i. hearing loss;
   ii. ototoxicity;
   iii. outer hair cell damage or death;
   iv loss of an outer hair cell; or
   v. a combination thereof;
b. decrease or eliminate
   i. the amount of an AMPK alpha gene product;
   ii. the amount of an CaMKKβ gene product;
   iii gene expression, protein expression, or both of AMPK alpha isoform 1, AMPK alpha isoform 2, or both;
   iv. gene expression, protein expression, or both of CaMKKβ; or
   v. a combination thereof, or
c. both

in the subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1D—Traumatic noise activates CaMKI in OHCs of the basal turn. (FIG. 1A) The p-CaMKI (Thr177, red) immunolabeling in OHCs of the basal turn was stronger examined 1 h after completion of 101-dB broad band noise (BBN) exposure than in unexposed controls. Phalloidin (green) staining for F-actin shows OHC structure. For better visualization of punctate labeling for p-CaMKI, one OHC was enlarged from the merged panel as indicated by the inset white box. These images were taken from the basal turn corresponding to the 22-32 kHz region and are representative of six mice in each group; scale bar=10 μm. (FIG. 1B) Semi-quantification of the p-CaMKI (Thr177) immunolabeling in OHCs in the 22-32 kHz region confirmed a significant increase. Data are presented as means+SD, n=6, ***p<0.001. (FIG. 1C) The basal region of the sensory epithelium displayed a tonotopic gradient with increasing numbers of OHCs positive for p-CaMKI (Thr177, red) immunolabeling farther from the apex 1 h after completion of exposure to 106-dB noise. The representative images were taken from the basal turn (lower base: 45-kHz, middle base: 32-kHz, and upper base: 22-kHz regions) and are representative of four mice for each group; scale bar=10 μm. (FIG. 1D) Counting the number of OHCs displaying p-CaMKI (Thr177) immunolabeling along the basal turn of the cochlear duct (3-5 mm from the apex) confirms a significant increase 1 h after completion of 106-dB noise exposure. Data are presented as means+SD, 106 dB: n=4; Control: n=6, *p<0.05.

FIGS. 2A-2B—Noise increases CaMKKβ expression in the cochlear outer hair cells. (FIG. 2A) Immunolabeling for CaMKKβ (red) in OHCs of the basal turn corresponding to sensitivity to 22-32 kHz was strong when processed 24 h after completion of the noise exposure compared to unexposed controls. Enlarged OHCs are shown for better visualization of punctate labeling for CaMKKβ; green: phalloidin-stained OHCs, scale bar=10 μm. (FIG. 2B) Semi-quantitative analysis of grayscale CaMKKβ labeling in OHCs confirmed a significant increase when examined at 24 h, but not at 1 h after the completion of the exposure. Data are presented as means+SD. The number of animals in each group is indicated in the bar graph, *p<0.05.

FIGS. 3A-3B—Treatment with KM plus furosemide increases CaMKKβ expression in the cochlear outer hair cells. (FIG. 3A) Immunolabeling for CaMKKβ (red) in OHCs of the basal turn corresponding to sensitivity to 22-32 kHz was strong when processed 24 h after KM plus furosemide treatment compared to untreated mice. KM alone or furosemide treatment alone showed similar CaMKKβ immunolabeling as those of control mice. Green: phalloidin-stained OHCs, scale bar=10 μm. (FIG. 3B) Semi-quantitative analysis of grayscale CaMKKβ labeling in OHCs confirmed a significant increase. Data are presented as means+SD; n=5 mice, *p<0.05.

FIGS. 4A-4D—Silencing efficiency between siCaMKKβ via intra-tympanic delivery onto the RWM and PSC delivery in OHCs. (FIG. 4A) Representative images show reduction of CaMKKβ-associated immunoreactivity in OHCs of the sensory epithelium 72 h after intra-tympanic delivery of siCaMKKβ onto the RWM of the left ears in mice when compared to those of scrambled RNA (siControl) mice. Images are representative of five individual mice for each condition. (FIG. 4B) Semi-quantification of CaMKKβ immunolabeling in grayscale in OHCs in the basal turn, corresponding to sensitivity to 22-32 kHz, confirms a significant decrease of about 40% in siCaMKKβ-treated cochleae compared to siControls. (FIG. 4C) Representative images show reduction of CaMKKβ-associated immunoreactivity in OHCs of the sensory epithelium 72 h after PSC delivery of siCaMKKβ to the left ears of mice when compared to those of siControl mice. Images are representative of five individual mice for each condition. Red: CaMKKβ; green: phalloidin-stained OHCs; scale bar=10 μm. (FIG. 4D) Semi-quantification of CaMKKβ immunolabeling in grayscale in OHCs in the basal turn, corresponding to sensitivity to 22-32 kHz, confirms a significant decrease of about 90% in siCaMKKβ-treated cochleae compared to siControls; siCaMKKβ: CaMKKβ siRNA, siControl: scrambled siRNA. Data are presented as means+SD, n=5 for each condition; **p<0.01, ****p<0.0001.

FIGS. 5A-5D—Pretreatment with CaMKKβ siRNA via intra-tympanic delivery on RWM reduces NIHL assessed 14 d after noise exposure. (FIG. 5A) Pretreatment with siCaMKKβ significantly decreases threshold shifts induced by exposure to 101-dB noise at 8, 16, and 32 kHz. Data are presented as means+SD, n=10 mice in each group. (FIG. 5B) Pretreatment with siCaMKKβ significantly attenuates 101-dB-noise-increased DPOAE thresholds from 12-40 kHz, n=5 mice in each group. (FIG. 5C) Representative images revealed Myosin-VIIA-immunolabeled OHCs (brown) 14 d after 101-dB-noise exposure in groups pretreated with siControl or siCaMKKβ. Images were taken at 4 mm from the apex. Scale bar=10 μm. (FIG. 5D) Quantification of OHC numbers along the cochlear duct showed that pretreatment with siCaMKKβ significantly reduced noise-induced OHC loss. siControl+101 dB: n=5; siCaMKKβ+101 dB noise: n=7. Data are presented as means±SD, *p<0.05, **p<0.01, ****p<0.001, ****p<0.0001.

FIGS. 6A-6B—Pretreatment with siCaMKKβ via intra-tympanic delivery onto the RWM attenuates noise-induced loss of synapses. (FIG. 6A) Representative images of immunolabeling for CtBP2 (red)/GluA2 (green) in the 22-kHz region of cochlear epithelia 14 d after completion of 101-dB-noise exposure. Images were compressed from 30 Z-stack projections. Scale bar=10 μm. (FIG. 6B) Quantification of CtBP2/GluA2-immunolabeled synapse particles in IHCs corresponding to 6, 8, 16, 22, and 32 kHz showed that broad band noise exposure reduced loss of synapses at 22 and 32 kHz, while pretreatment with siCaMKKβ prevented noise-induced loss of synapses. The frequency correlation along the cochlear epithelium is indicated at the bottom. Data are presented as means±SD, n=5 in each noise-exposed group with siControl and siCaMKKβ, n=10 control mice without noise exposure, ****p<0.001.

FIGS. 7A-7C—A mouse model of kanamycin-plus-furosemide-induced hearing loss in CBA/J mice at the age of 6-7 weeks. A single treatment of kanamycin (KM) at 800 mg/kg subcutaneously (SQ) followed by furosemide (Fu) at 200 mg/kg via intraperitoneal (IP) injection resulted in permanent auditory threshold shifts at 8, 16, and 32 kHz when measured 14 d after the injections. Data are presented as mean±SD, ****p<0.0001. One-time injection of KM or Fu alone does not induce hearing loss at any measured frequency.

FIGS. 8A-8D—Pretreatment with CaMKKβ siRNA via intra-tympanic delivery onto the RWM attenuates auditory threshold shifted by co-treatment of KM with furosemide; pretreatment via PSC application completely prevents KM-furosemide-induced auditory threshold shifts and damage to OHC function when measured 14 d after the injection. (FIGS. 8A-8C) Pre-application of siCaMKKβ through delivery onto the RWM resulted in significant reduction of KM-furosemide-induced auditory threshold shifts at 8, 16, and 32 kHz. Surprisingly, delivery of siCaMKKβ through the PSC completely prevented KM-furosemide-induced-auditory threshold shifts at all measured frequencies (8, 16, and 32 kHz). (FIG. 8D) OHC functional measurement by DPOAE showed that pretreatment with siCaMKKβ via the PSC also completely prevented reduction of DPOAE by co-treatment of KM-furosemide. Data are presented as means+SEM, Naive: n=13, siControl+KM-furosemide: n=8, siCaMKKβ+KM-furosemide: n=7. All ARBs and DPOAEs were measured on the left ears of mice. Statistical analysis between siCtrl+KM+Fu and siCaMKKβ+KM+Fu were analyzed by repeated measures ANOVA. F 1, 10=87.831, p<0.0001.

FIGS. 9A-9C—Pretreatment with CaMKKβ siRNA via PSC application completely prevents KM-furosemide-induced OHC loss when measured 14 d after the lesions. (FIGS. 9A-9B) Representative images of the apex, middle, and basal turns display myosin-VIIa-labeled sensory hair cells in siCtrl+KM+Fu and siCaMKKβ+KM+Fu groups by 10× magnification (FIG. 9A) or 63× magnifications, scale bar=10 μm (FIG. 9B). (FIG. 9C) Hair cells were counted along the entire length of the cochlear spiral. The distances along the cochlear duct that correlate with the frequencies of 8, 16, and 32 kHz are indicated. Data are presented as means±SEM and analyzed by repeated measures ANOVA. F_{1, 13}=742.84, p<0.0001. One cochlea was used per mouse.

FIGS. 10A-10F—Mouse model of kanamycin-plus-furosemide-induced hearing loss in CBA/J mice at the age of 6-7 weeks. (FIGS. 1A-10C) A single treatment of kanamycin (KM) at 800 mg/kg SQ plus furosemide (Fu) at 100 mg/kg via IP injection resulted in auditory threshold shifts of 20-30 dB at 8, 16, and 32 kHz when measured 14 d after the injections. (FIGS. 10D-10F) A single treatment of KM at 800 mg/kg SQ plus furosemide at 200 mg/kg via IP injection resulted in auditory threshold shifts of 50-60 dB at 8, 16, and 32 kHz when measured 14 d after the treatment. Data are presented as individual points with means+SD. These results can demonstrate that the dose of KM at 800 mg/kg plus furosemide at 200 mg/kg is suitable for our investigation.

FIGS. 11A-11B—Treatment with KM plus furosemide increases activation of AMPKα in cochlear outer hair cells. (FIG. 11A) Immunolabeling for p-AMPKα (red) in OHCs of the basal turn corresponding to sensitivity to 22-32 kHz was strong when processed 24 h after KM plus furosemide treatment compared to untreated mice. Green: phalloidin-stained OHCs, scale bar=10 μm. (FIG. 11B) Semi-quantitative analysis of immunolabeling for p-AMPKα (converted in grayscale) in OHCs confirmed a significant increase. Data are presented as means+SD; n=5 mice, **p<0.01. These results indicate that KM-furosemide treatment induces activation of AMPKα in OHCs.

FIGS. 12A-12F—Pretreatment with AMPKα siRNA via intra-tympanic delivery onto the RWM attenuates auditory threshold shifts from treatment of KM followed by furosemide with large variation, significant at 32 kHz (FIGS. 12A-12C). Pretreatment with AMPKα siRNA via PSC application completely prevents KM-furosemide-induced auditory threshold shifts at all three measured frequencies when measured 14 d after the injection (FIGS. 12D-12F). All ARBs were measured on the left ears of mice. Data are presented as individual points with means±SD, **p<0.01, ****p<0.0001.

FIG. 13—Delivery of AMPKα siRNA via PSC application completely prevents KM-furosemide-induced damage to OHC function measured by DPOAE 14 d after the injection. Data are presented as means±SD; Naive: n=13, siControl+KM-furosemide: n=6, siAMPKα+KM-furosemide: n=6. DPOAEs were measured on the left ears of the mice. Statistical analysis between siCtrl+KM+Fu and siAMPKα+KM+Fu were performed by repeated measures ANOVA. F 1, 9=389.18, p<0.0001.

FIGS. 14A-14C—Pretreatment with AMPKα siRNA via PSC application completely prevents KM-furosemide-induced OHC loss when measured 14 d after the lesions. (FIG. 14A-14B) Representative images of the apex, middle, and basal turns display myosin-VIIa-labeled sensory hair cells in siCtrl+KM+Fu and siAMPKα+KM+Fu groups by 10× (A) and 63× magnifications (FIG. 14B). (FIG. 14C) Hair cells were counted along the entire length of the cochlear spiral. The distances along the cochlear duct that correlate with the frequencies of 8, 16, and 32 kHz are indicated. Data are presented as means±SD and analyzed by repeated measures ANOVA. F1, 14=1279.06, p<0.0001. One cochlea was used per mouse.

FIGS. 15A-15F—Mouse model of cisplatin-plus-furosemide-induced hearing loss in CBA/J mice at the age of 6-7 weeks. (FIGS. 15A-15C) Treatment with cisplatin (CDDP) at doses from 2-4 mg/kg plus furosemide at 200 mg/kg via IP injection for 3 consecutive days resulted in auditory threshold shifts measured at 8, 16, 32 kHz when measured on day 7 after treatment. (FIGS. 15D-15F) Auditory threshold shifts were maintained at 8, 16, 32 kHz when measured on day 14 after the treatment. Data are presented as individual points with means SD.

FIGS. 16A-16B—Treatment with CDDP/furosemide causes massive loss of outer hair cells (OHCs) in mice when examined 14 d after the treatment. (FIG. 16A) Representative images of myosin-VIIa-labeled and DAB-stained apical, middle, and basal turns of surface preparations treated with 2.5 mg/kg CDDP. Scale bar=20 μm. (FIG. 16B) Quantitative analysis of OHC loss revealed complete loss of OHCs along the entire length of cochlear spiral with CDDP at 4 mg/kg plus furosemide at 200 mg/kg treatment and complete loss of OHCs in middle and basal turns with CDDP doses from 2.5-3 mg/kg. Data are presented as means SD; CDDP 2.5 mg/kg: n=5, CDDP 3 mg/kg: n=2, CDDP 4 mg/kg: n=3.

FIG. 17—Changes in body weight with difference doses of CDDP treatment. Body weight was measured before treatment, on treatment days, and 7 and 14 d post treatment. There are no significant changes in body weight with treatment of CDDP at doses below or equal 2.5 mg/kg, but loss of body weight appeared on day 7 post CDDP treatment at and above 3 mg/kg doses compared with beginning of the treatment. Data are presented as means±SD. CDDP2 mg/kg: n=2, CDDP 2.5 mg/kg: n=5, CDDP 3 mg/kg: n=2, CDDP 4 mg/kg: n=3.

FIGS. 18A-18C—Pretreatment with AMPKα siRNA (0.6 μg) via PSC application completely prevented CDDP-induced auditory threshold shifts at 8 and 16 kHz when measured on day 3 post (FIG. 18A) day 7 post (FIG. 18B) as well as day 14 post (FIG. 18C) compared with scrambled (siControl) mice. CDDP-induced auditory threshold shifts were partially attenuated at 32 kHz with huge variations between each mouse. CDDP was used at 2.5 mg/kg plus 200 mg/kg furosemide. Data are presented as individual points with means±SD, **p<0.01, ****p<0.0001.

FIGS. 19A-19B—Pretreatment with AMPKα siRNA via PSC application significantly attenuated CDDP/furosemide-induced outer hair cell loss when measured 14 d after the treatment. (A) Representative images from basal turn of surface preparations corresponding to sensitivity at 32 kHz region show myosin-VIIa-labeled and DAB-stained sensory hair cells in siControl+CDDP and siAMPKα+CDDP. CDDP: 2.5 mg/kg; furosemide: 200 mg/kg; Scale bar=20 μm. (B) Outer hair cells were counted along the entire length of the cochlear spiral revealing significant protection from hair cell damage with AMPKα siRNA treatment. Data are presented as means±SD, n=5 in each group.

FIGS. 20A-20B—Pretreatment with siCaMKKβ via the PSC does not change FM1-43 uptake into OHCs compared with that of the siControl mice. FIG. 20A shows images of green fluorescence that reveal uptake of FM1-43 in OHCs. Scale bar=10 μm. FIG. 20B demonstrates a semi-quantitative analysis of the grayscales of green fluorescence intensity that shows no significant difference between siControl and siCaMKKβ groups. Data are presented as means+SD; ns: not significant.

FIGS. 21A-21B—Pretreatment with siAMPKα1 via the PSC does not change FM1-43 uptake into OHCs compared with that of the siControl mice. FIG. 21A shows Images of green fluorescence reveal uptake of FM1-43 in OHCs. Scale bar=10 μm. FIG. 21B shows a semi-quantitative analysis of the grayscales of green fluorescence intensity shows no significant difference between siControl and siAMPKα groups. Data are presented as means+SD; ns: not significant.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” refers to an administration that is auricular (otic), oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “agent” refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “antibody” can refer to a glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies.

As used herein, As used herein, “anti-infective” refers to compounds or molecules that can either kill an infectious agent or inhibit it from spreading. Anti-infectives include, but are not limited to, antibiotics, antibacterials, antifungals, antivirals, and antiprotozoans.

As used herein, “aptamer” can refer to single-stranded DNA or RNA molecules that can bind to pre-selected targets including proteins with high affinity and specificity. Their specificity and characteristics are not directly determined by their primary sequence, but instead by their tertiary structure.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “dose,” “unit dose,” or “dosage” can refer to physically discrete units suitable for use in a subject, each unit containing a predetermined quantity of the compounds of the present invention and/or a pharmaceutical formulation thereof calculated to produce the desired response or responses in association with its administration.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also reflect the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, the terms “Fc portion,” “Fc region,” and the like are used interchangeable herein and can refer to the fragment crystallizable region of an antibody that interacts with cell surface receptors called Fc receptors and some proteins of the complement system. The IgG Fc region is composed of two identical protein fragments that are derived from the second and third constant domains of the IgG antibody's two heavy chains.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (MW) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphonothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “ototoxic,” “ototoxicity” and the like refers to the state or property of being toxic the ear or a component thereof, including but not limited to the cochlea, auditory nerve, vestibular system. Ototoxicity can be reversible and temporary or irreversible and permanent.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “preventative” and “prevent” refers to hindering or stopping a disease or condition before it occurs, even if undiagnosed, or while the disease or condition is still in the sub-clinical phase.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as, but not limited to, reduction of CMKKbeta gene product amount and/or translation, hair cell (e.g. an outer hair cell) damage and/or death, hearing loss, and/or auditory nerve damage and/or death. The effect can be therapeutic in terms of a partial or complete cure of a disease, condition, symptom or adverse effect attributed to the disease, disorder, or condition. The term “treatment” as used herein covers any treatment of auditory cell, hair cell (e.g. an outer hair cell) damage and/or death, hearing loss, and/or auditory nerve damage and/or death in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt. %,” and “wt. %,” which can be used interchangeably, indicate the percent by weight of a given component based on the total weight of a composition of which it is a component, unless otherwise specified. That is, unless otherwise specified, all wt. % values are based on the total weight of the composition. It should be understood that the sum of wt. % values for all components in a disclosed composition or formulation are equal to 100. Alternatively, if the wt. % value is based on the total weight of a subset of components in a composition, it should be understood that the sum of wt. % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Acquired hearing loss is caused by numerous etiologies accumulated over a lifetime, such as exposure to excessive noise, exposure to ototoxic agents (e.g., medications such as aminoglycoside antibiotics and the anti-cancer drug cisplatin), bacterial or viral ear infections, head injuries, and the aging process. Ototoxicity by ototoxic agents can also be referred to herein as “chemically-induced hearing loss” when the ototoxic agent is a chemical compound or agent.

The common pathology of acquired hearing loss is loss of cochlear sensory hair cells with outer hair cells (OHCs) being more sensitive than inner hair cells (IHCs). This common pathology has been well documented both in humans and in animal models. Since cochlear sensory hair cells in mammals do not regenerate, the loss is irreversible and causes permanent hearing loss.

The Cartcalmodulin-dependent protein kinase kinases (CaMKKs) are serine/threonine-directed protein kinases that are activated following increases in intracellular Ca2+ concentration. CaMKKα and CaMKKβ are the two isoforms, of which CaMKKβ is the main isoform in brain tissues. Noise- and aminoglycoside-induced calcium (Ca2+) influx in outer hair cells (OHCs) has been well-documented in the pathogenesis of noise-induced hearing loss (NIHL) and aminoglycoside-induced hearing loss. Loss of sensory hair cells is a common pathological feature of inner ear damage, including from noise trauma and aminoglycoside exposure, with OHCs being more vulnerable than inner hair cells and following base-to-apex gradient of sensory hair cell loss (Forge & Schacht, 2000; Sha & Schacht, 2017). Noise exposure can result in activation of AMPKα in OHCs (Chen et al., 2012; Hill et al., 2016). CaMKKβ is one of the major upstream activators of the AMP-dependent protein kinase (AMPK). AMPK is a protein that contains alpha, beta, and gamma subunits. The alpha subunit has at least two isoforms, isoform 1 and 2, that are expressed in sensory hair cells (Hill et al. 2016).

Cisplatin (CDDP), a widely-used cancer treatment, causes major toxic effects, such as nephrotoxicity, liver toxicity, and ototoxicity (Hong, Hwang et al. 2005, Barabas, Milner et al. 2008, Karasawa and Steyger 2015, Crona, Faso et al. 2017). Among these toxic effects, CDDP-induced ototoxicity is irreversible resulting in sensorineural deafness in both ears (Knight, Kraemer et al. 2005, Barabas, Milner et al. 2008). Clinically, although older people are more prone to ototoxicity and severe hearing impairment; children are more sensitive to CDDP-induced ototoxicity (MURAKAMI, INOUE et al. 1990, Paken, Govender et al. 2016). The main pathological feature of CDDP-induced hearing loss is loss of cochlear sensory hair cells in a base-to-apex gradient with loss beginning at the basal turn of the cochlear epithelium (Alam, Ikeda et al. 2000, Frisina, Wheeler et al. 2016, Paken, Govender et al. 2016). Since mammalian auditory sensory hair cells lack the ability to regenerate, loss of hair cells is permanent. Currently, there is no pharmaceutical drug available to prevent or alleviate CDDP-induced hair cell loss and hearing loss. In order to prevent cisplatin-induced ototoxicity, many potential mechanisms have been investigated in CDDP-induced hearing loss, finding an increase in intracellular reactive oxygen species (ROS) and decrease in the antioxidant defense system (Kopke, Liu et al. 1997, Pabla and Dong 2008, Chirino and Pedraza-Chaverri 2009). Some apoptosis related signaling pathways have also been shown to be activated in CDDP-induced apoptosis, such as caspase and TNFα pathways (Benkafadar, Menardo et al. 2017, Xu, Ma et al. 2017, Ruhl, Du et al. 2019).

There is currently no clinically pharmaceutical therapies for prevention and treatment of acquired hearing loss, let alone any that are capable of targeting CaMKKβ and/or AMPK.

With this in mind, embodiments disclosed herein can provide compositions, pharmaceutical formulations that can target one or more genes and/or gene products involved in the development of acquired hearing loss. In some embodiments, the gene that can be targeted is CaMKKβ and/or AMPKα. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### CAMKK Beta Targeting Polynucleotides

As is also demonstrated in the Examples herein, noise-exposure-increased CaMKKβ expression in OHCs can result in increased phosphorylated CaMKI (p-CaMKI)-positive OHCs in the basal region of the cochlea following a base-to-apex gradient as soon as 1 hour after exposure. Pretreatment with CaMKKβ siRNA (siCaMKKβ) via RWM application reduced CaMKKβ in OHCs and significantly attenuated noise-induced losses of synapses and OHCs and NIHL. As such CaMKKβ can be a target for prevention and treatment of acquired hearing loss.

Described herein are various embodiments of CaMKKβ targeting molecules (e.g. targeting polynucleotides) that are, in some embodiments, capable of targeting CaMKKβ and inhibiting its expression and/or reducing a CaMKKβ gene product (e.g. an RNA transcript and/or protein). Reduction of CaMKKβ expression and/or a CaMKKβ gene product can prevent, treat or otherwise mitigate acquired hearing loss. Also described elsewhere herein are vectors, formulations, such as pharmaceutical formulations, that can contain one or more CaMKKβ targeting compositions described herein.

Described herein are various embodiments of CaMKKβ targeting molecules (such as CaMKKβ targeting RNAi molecules) that include or be composed only of a targeting sequence capable of specifically targeting the targeting molecule to a CaMKKβ gene product. In some embodiments, the CaMKKβ targeting molecule (such as a CaMKKβ targeting RNAi molecule) can be a polynucleotide. The polynucleotide can be RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), piRNA (piwi-interacting RNA), or ribozymes, aptamers, or mRNA (messenger RNA). In some embodiments, the CaMKKβ gene product (which is targeted for example) is an RNA molecule transcribed from the CaMKKβ gene. In some embodiments, CaMKKβ gene product is mRNA.

In certain example embodiments, the CaMKKβ targeting molecule (such as a CaMKKβ targeting RNAi molecule) is capable of reducing or eliminating the amount and/or translation of the CaMKKβ gene product. In some embodiments, reduction or elimination can occur via RNAi machinery within a cell to which the CaMKKβ targeting molecule is be delivered. RNAi machinery and mechanisms of action which lead to a reduction or elimination of the presence or expression of a gene or gene product are generally known in the art. See e.g., Han H. 2018. Methods Mol Biol. 1706:293-302; Chen et al. 2018. Cancer Metastasis Rev. 37(1):107-124; Dong et al., 2019. Adv. Drug Deliv Rev. 144:133-147; and Bhattacharjee et al. 2019. RNA Biol. 16(9)1133-1146). Methods and techniques of determining and measuring the amount of CaMKKβ gene product are generally known to one of ordinary skill in the art and can include, but are not limited to, PCR-based techniques (e.g. RT-PCR, RT-qPCR, RNA Seq, and the like). In some embodiments, the polynucleotide is capable of reducing or eliminating the amount and/or a function of a CaMKKβ polypeptide. Methods and techniques of measuring protein expression and function are generally known to one of ordinary skill in the art and can include, but are not limited to, Western analysis, immunohistochemistry, ELISA, mass-spectrometry analysis, and protein sequencing.

CaMKKβ (calcium/calmodulin-dependent protein kinase kinase beta) plays a role in calcium/calmodulin dependent (CaM) kinase cascade by phosphorylating the downstream kinases CaMK1 and CaMK4. It also regulates the production of neuropeptide Y and can function as an AMPK kinase. At least seven transcript variants that encode six isoforms have been identified. Several splice variants have also been identified (Hsu L S, Chen G D, Lee L S, Chi C W, Cheng J F, Chen J Y August 2001). “Human Ca²⁺/calmodulin-dependent protein kinase kinase beta gene encodes multiple isoforms that display distinct kinase activity”. J. Biol. Chem. 276 (33): 31113-23. A reference hCaMKKβ gene can be Entrez Gene ID No.: 10645 reference hCaMKKβ gene product can be GenBank Accession No. NM_001270485, NM_001270486, NM 006549, NM_153499, or NM_153500. A reference mCaMKKβ gene can be Entrez Gene ID No.: 207565. A reference mCaMKKβ gene product can be GenBank Accession No. NM_001199676 or NM_145358.

In some embodiments, the targeting sequence can be 50-100% identical to a sequence that is complementary to a target sequence in the CaMKKβ gene product. In some embodiments, the targeting sequence can be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% identical to a sequence that is complementary to a target sequence in the CaMKKβ gene product. As used herein, “identity,” can refer to a relationship between two or more nucleotide or polypeptide sequences, as determined by comparing the sequences. In the art, “identity” refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides or polynucleotides of the present disclosure, unless stated otherwise.

In some embodiments, the targeting sequence is capable of specifically binding a target sequence in the CaMKKβ gene product.

In some embodiments, the target sequence is 50-100% identical to any 4-500 consecutive nucleotides in a CAMKKβ gene product. In some embodiments, the target sequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% identical to any 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 consecutive nucleotides in a CAMKKβ gene product. In some embodiments, the gene product can be 50-100% identical to a reference CAMKKβ gene product. Exemplary reference CAMKKβ gene products are previously described. In some embodiments, the target sequence can be selected from the group comprising any one or more of those provided in Table 1 or a combination thereof. In some embodiments, the CaMKKβ-targeting polynucleotide has a sequence according to any of those provided in Table 1 or a combination thereof. In some embodiments,

TABLE 1

| TRC | | SEQ ID | | |
|---|---|---|---|---|
| Number | Region | NO: | Sequence | Species |
| TRCN0000363121 | CDS | 3 | CCGGGCCATGGGTGTGACACTATACCTCGAGG | Human |
| | | | TATAGTGTCACACCCATGGCTTTTTG | |
| TRCN0000195473 | 3UTR | 4 | CCGGCCCGATGCTTCTGTTTCATTCCTCGAGG | Human |
| | | | AATGAAACAGAAGCATCGGGTTTTTTG | |
| TRCN0000196992 | 3UTR | 5 | CCGGGCAAAGAGGACGCCCATAATTCTCGAGA | Human |
| | | | ATTATGGGCGTCCTCTTTGCTTTTTTG | |
| TRCN0000199090 | CDS | 6 | CCGGCCTCTCATCCTTGAGCATCCACTCGAGT | Human |
| | | | GGATGCTCAAGGATGAGAGGTTTTTTG | |
| TRCN0000363122 | 3UTR | 7 | CCGGATCGTCATCTCTGGTTATTTGCTCGAGC | Human |
| | | | AAATAACCAGAGATGACGATTTTTTG | |
| TRCN0000363096 | CDS | 8 | CCGGTCGTCAAGTTGGCCTACAATGCTCGAGC | Human |
| | | | ATTGTAGGCCAACTTGACGATTTTTG | |
| TRCN0000194684 | CDS | 9 | CCGGCAATACCTACTATGCAATGAACTCGAGT | Human |
| | | | TCATTGCATAGTAGGTATTGTTTTTTG | |
| TRCN0000002297 | CDS | 10 | CCGGCGAGCGGATCATGTGTTTACACTCGAGT | Human |
| | | | GTAAACACATGATCCGCTCGTTTTT | |
| TRCN0000002298 | CDS | 11 | CCGGCCGTTTCTACTTCCAGGATCTCTCGAGA | Human |
| | | | GATCCTGGAAGTAGAAACGGTTTTT | |
| TRCN0000002299 | CDS | 12 | CCGGGTGAAGACCATGATACGTAAACTCGAGT | Human |
| | | | TTACGTATCATGGTCTTCACTTTTT | |
| TRCN0000002301 | 3UTR | 13 | CCGGCGACCCTTTCTACTATGCATTCTCGAGA | Human |
| | | | ATGCATAGTAGAAAGGGTCGTTTTT | |
| TRCN0000002300 | CDS | 14 | CCGGAGTCAAACACATTCCCAGCTTCTCGAGA | Human |
| | | | AGCTGGGAATGTGTTTGACTTTTTT | |
| TRCN0000276712 | CDS | 15 | CCGGCCATGATTCGAAAGCGCTCATCTCGAGA | Mouse |
| | | | TGAGCGCTTTCGAATCATGGTTTTTG | |
| TRCN0000276649 | CDS | 16 | CCGGGTATCCACTTGGGCATGGAATCTCGAGA | Mouse |
| | | | TTCCATGCCCAAGTGGATACTTTTTG | |
| TRCN0000276714 | CDS | 17 | CCGGTTTCCCGACCAGCCCGATATACTCGAGT | Mouse |
| | | | ATATCGGGCTGGTCGGGAAATTTTTG | |
| TRCN0000028815 | CDS | 18 | CCGGCGGTGTAAGCAACGAGTTCAACTCGAGT | Mouse |
| | | | TGAACTCGTTGCTTACACCGTTTTT | |
| TRCN0000028809 | CDS | 19 | CCGGGCTGAATCAGTACACCCTGAACTCGAGT | Mouse |
| | | | TCAGGGTGTACTGATTCAGCTTTTT | |
| TRCN0000028776 | CDS | 20 | CCGGCCATGATTCGAAAGCGCTCATCTCGAGA | Mouse |
| | | | TGAGCGCTTTCGAATCATGGTTTTT | |
| TRCN0000028764 | CDS | 21 | CCGGGTATCCACTTGGGCATGGAATCTCGAGA | Mouse |
| | | | TTCCATGCCCAAGTGGATACTTTTT | |
| TRCN0000028761 | CDS | 22 | CCGGCCCTTTCATGGATGAACGAATCTCGAGA | Mouse |
| | | | TTCGTTCATCCATGAAAGGGTTTTT | |
| TRCN0000281992 | 3UTR | 23 | CCGGGACTCCATGTCGTCGACTTTGCTCGAGC | Mouse |
| | | | AAAGTCGACGACATGGAGTCTTTTTG | |
| TRCN0000276713 | CDS | 24 | CCGGCGGTGTAAGCAACGAGTTCAACTCGAGT | Mouse |
| | | | TGAACTCGTTGCTTACACCGTTTTTG | |

The targeting polynucleotide can have 4 to 500 or more nucleotides. In some embodiments, the targeting polynucleotide can have 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

Also described herein are polynucleotides capable of encoding the CaMKKβ targeting molecule (such as a polynucleotide). As used herein, the term “encode” refers to principle that DNA can be transcribed into RNA, which can then be translated into amino acid sequences that can form proteins.

### AMPK Alpha Targeting Polynucleotides

Described herein are various embodiments of AMPKα targeting polynucleotides that are, in some embodiments, capable of targeting an AMPKα isoform (referred to collectively herein as “an AMPKα”) (e.g. AMPKα-1 and/or AMPKα-2) and inhibiting the expression of and/or reducing the amount of an AMPKα gene product (e.g. an RNA transcript and/or protein). Reduction of AMPKα and/or it's gene product can prevent, treat or otherwise mitigate acquired hearing loss. Also described herein are vectors, formulations, such as pharmaceutical formulations, that can contain one or more AMPKα targeting compositions described herein.

Described herein are various embodiments of AMPKα targeting molecules (such as AMPKα targeting RNAi molecules) that include or be composed only of a targeting sequence capable of specifically targeting the targeting molecule to an AMPKα gene product. In some embodiments, the AMPKα targeting molecule (such as an AMPKα targeting RNAi molecule) can be a polynucleotide. The polynucleotide can be RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), piRNA (piwi-interacting RNA), or ribozymes, aptamers, or mRNA (messenger RNA). In some embodiments, the AMPKα gene product is an RNA molecule transcribed from the AMPKα gene. In some embodiments, the AMPKα gene product is mRNA.

In certain example embodiments, the AMPKα targeting molecule (such as an AMPKα targeting RNAi molecule) is capable of reducing or eliminating the amount and/or translation of the AMPKα gene product. In some embodiments, this can occur via RNAi machinery within a cell to which the AMPKα targeting molecule is be delivered. RNAi machinery and mechanisms of action which lead to a reduction or elimination of the presence or expression of a gene or gene product are generally known in the art. See e.g., Han H. 2018. Methods Mol Biol. 1706:293-302; Chen et al. 2018. Cancer Metastasis Rev. 37(1):107-124; Dong et al., 2019. Adv. Drug Deliv Rev. 144:133-147; and Bhattacharjee et al. 2019. RNA Biol. 16(9)1133-1146). Methods and techniques of determining and measuring the amount of AMPKα gene product are generally known to one of ordinary skill in the art and can include, but are not limited to, PCR-based techniques (e.g. RT-PCR, RT-qPCR, RNA Seq, and the like). In some embodiments, the polynucleotide is capable of reducing or eliminating the amount and/or a function of an AMPKα polypeptide. Methods and techniques of measuring protein expression and function are generally known to one of ordinary skill in the art and can include, but are not limited to, Western analysis, immunohistochemistry, ELISA, mass-spectrometry analysis, and protein sequencing.

AMPKα (5′ AMP-activated protein kinase) plays a role in cellular energy homeostasis and can be phosphorylated by CaMKKβ. AMPK exists as composed of a catalytic alpha subunit, and regulatory beta and gamma subunits. In humans, each of these occurs as multiple isoforms encoded by distinct genes. AMPKα genes are discussed in greater detail elsewhere herein. Genes encoding the three subunits can also be sound in the genomes of almost all eukaryotes, form single celled protozoa to humans.

In some embodiments, the targeting sequence can be 50-100% identical to a sequence that is complementary to a target sequence in the AMPKα gene product. In some embodiments, the targeting sequence can be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% identical to a sequence that is complementary to a target sequence in the AMPKα gene product. As used herein, “identity,” can refer to a relationship between two or more nucleotide or polypeptide sequences, as determined by comparing the sequences. In the art, “identity” refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides or polynucleotides of the present disclosure, unless stated otherwise.

In some embodiments, the targeting sequence is capable of specifically binding a target sequence in the AMPKα gene product.

In some embodiments, the target sequence is 50-100% identical to any 4-500 consecutive nucleotides in an AMPKα gene product. In some embodiments, the target sequence is 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% identical to any 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 consecutive nucleotides in an AMPKα gene product. In some embodiments, the gene product can be 50-100% identical to a reference AMPKα gene product or 50-100% identical to a sequence that is complementary to a sequence in a reference AMPKα gene product. Exemplary reference AMPKα gene products are previously described and include but are not limited to AMPKα-1 and AMPKα-2 DNA, RNA, and/or polypeptides. In some embodiments, the target sequence can be selected from the group comprising any one or more of those sequences of those provided in Table 2 or a combination thereof. In some embodiments, the AMPKα-targeting molecule has a sequence according to any of those provided in Table 2 or a combination thereof.

TABLE 2

| | SEQ | | | |
|---|---|---|---|---|
| TRC | ID | | | |
| Number | NO: | Region | Sequence | SPECIES |
| PRKAA1 | | | | |
| TRCN0000360841 | 25 | CDS | CCGGTTGTTGGATTTCCGTAGTATTCTCGAGAATA | Mouse |
| | | | CTACGGAAATCCAACAATTTTTG | |
| TRCN0000360770 | 26 | 3UTR | CCGGGAATCCTCATAGACCTTATTACTCGAGTAAT | Mouse |
| | | | AAGGTCTATGAGGATTCTTTTTG | |
| TRCN0000360769 | 27 | 3UTR | CCGGGACCATAAATTTACCATAAAGCTCGAGCTTT | Mouse |
| | | | ATGGTAAATTTATGGTCTTTTTG | |
| TRCN0000360842 | 28 | CDS | CCGGCACGAGTTGACCGGACATAAACTCGAGTTTA | Mouse |
| | | | TGTCCGGTCAACTCGTGTTTTTG | |
| TRCN0000023999 | 29 | CDS | CCGGCCCATCTTATAGTTCAACCATCTCGAGATGG | Mouse |
| | | | TTGAACTATAAGATGGGTTTTT | |
| TRCN0000024000 | 30 | CDS | CCGGGCAATCAAGCAGTTGGATTATCTCGAGATAA | Mouse |
| | | | TCCAACTGCTTGATTGCTTTTT | |
| TRCN0000024001 | 31 | CDS | CCGGCCTGAATCGAAATGTGTTCTTCTCGAGAAGA | Mouse |
| | | | ACACATTTCGATTCAGGTTTTT | |
| TRCN0000024002 | 32 | CDS | CCGGCGTAGTATTGATGATGAGATTCTCGAGAATC | Mouse |
| | | | TCATCATCAATACTACGTTTTT | |
| TRCN0000024003 | 33 | CDS | CCGGCCACAGAAATCCAAACACCAACTCGAGTTGG | Mouse |
| | | | TGTTTGGATTTCTGTGGTTTTT | |
| TRCN0000196482 | 34 | CDS | CCGGGTAGCTGTGAAGATACTCAATCTCGAGATTG | Mouse |
| | | | AGTATCTTCACAGCTACTTTTTTG | |
| TRCN0000219690 | 35 | CDS | CCGGGAAGGTTGTAAACCCATATTACTCGAGTAAT | Mouse |
| | | | ATGGGTTTACAACCTTCTTTTTG | |
| TRCN0000199831 | 36 | CDS | CCGGGTGACCTCACTTGACTCTTCTCTCGAGAGAA | Mouse |
| | | | GAGTCAAGTGAGGTCACTTTTTTG | |
| TRCN0000196831 | 37 | 3UTR | CCGGGCACAGACAATTGCAGTAAATCTCGAGATTT | Mouse |
| | | | ACTGCAATTGTCTGTGCTTTTTTG | |
| TRCN0000000857 | 38 | 3UTR | CCGGGCATAATAAGTCACAGCCAAACTCGAGTTTG | Mouse |
| | | | GCTGTGACTTATTATGCTTTTT | |
| TRCN0000000858 | 39 | CDS | CCGGCCATCCTGAAAGAGTACCATTCTCGAGAATG | Mouse |
| | | | GTACTCTTTCAGGATGGTTTTT | |
| TRCN0000000860 | 40 | CDS | CCGGTGATTGATGATGAAGCCTTAACTCGAGTTAA | Mouse |
| | | | GGCTTCATCATCAATCATTTTT | |
| TRCN0000000859 | 41 | CDS | CCGGCCTGGAAGTCACACAATAGAACTCGAGTTCT | Mouse |
| | | | ATTGTGTGACTTCCAGGTTTTT | |
| TRCN0000000861 | 42 | CDS | CCGGGTTGCCTACCATCTCATAATACTCGAGTATT | Mouse |
| | | | ATGAGATGGTAGGCAACTTTTT | |
| TRCN0000219691 | 43 | CDS | CCGGCTACTGGATTTCCGTAGTATTCTCGAGAATA | Mouse |
| | | | CTACGGAAATCCAGTAGTTTTTG | |
| TRCN0000196482 | 44 | CDS | CCGGGTAGCTGTGAAGATACTCAATCTCGAGATTG | Human |
| | | | AGTATCTTCACAGCTACTTTTTTG | |
| TRCN0000219690 | 45 | CDS | CCGGGAAGGTTGTAAACCCATATTACTCGAGTAAT | Human |
| | | | ATGGGTTTACAACCTTCTTTTTG | |
| TRCN0000199831 | 46 | CDS | CCGGGTGACCTCACTTGACTCTTCTCTCGAGAGAA | Human |
| | | | GAGTCAAGTGAGGTCACTTTTTTG | |
| TRCN0000196831 | 47 | 3UTR | CCGGGCACAGACAATTGCAGTAAATCTCGAGATTT | Human |
| | | | ACTGCAATTGTCTGTGCTTTTTTG | |
| TRCN0000000857 | 48 | 3UTR | CCGGGCATAATAAGTCACAGCCAAACTCGAGTTTG | Human |
| | | | GCTGTGACTTATTATGCTTTTT | |
| TRCN0000000858 | 49 | CDS | CCGGCCATCCTGAAAGAGTACCATTCTCGAGAATG | Human |
| | | | GTACTCTTTCAGGATGGTTTTT | |
| TRCN0000000860 | 50 | CDS | CCGGTGATTGATGATGAAGCCTTAACTCGAGTTAA | Human |
| | | | GGCTTCATCATCAATCATTTTT | |
| TRCN0000000859 | 51 | CDS | CCGGCCTGGAAGTCACACAATAGAACTCGAGTTCT | Human |
| | | | ATTGTGTGACTTCCAGGTTTTT | |
| TRCN0000000861 | 52 | CDS | CCGGGTTGCCTACCATCTCATAATACTCGAGTATT | Human |
| | | | ATGAGATGGTAGGCAACTTTTT | |
| TRCN0000219691 | 53 | CDS | CCGGCTACTGGATTTCCGTAGTATTCTCGAGAATA | Human |
| | | | CTACGGAAATCCAGTAGTTTTTG | |
| PRKAA2 | | | | |
| TRCN0000024044 | 54 | CDS | CCGGGCCCAGATGAACGCTAAGATACTCGAGTATC | Mouse |
| | | | TTAGCGTTCATCTGGGCTTTTT | |
| TRCN0000024047 | 55 | CDS | CCGGCAGGCCATAAAGTGGCAGTTACTCGAGTAAC | Mouse |
| | | | TGCCACTTTATGGCCTGTTTTT | |
| TRCN0000024048 | 56 | CDS | CCGGCCCAAGTCTCTAGCTGTGAAACTCGAGTTTC | Mouse |
| | | | ACAGCTAGAGACTTGGGTTTTT | |
| TRCN0000024046 | 57 | CDS | CCGGCGCCAGTCTTATCACTGCTTTCTCGAGAAAG | Mouse |
| | | | CAGTGATAAGACTGGCGTTTTT | |
| TRCN0000024045 | 58 | CDS | CCGGCGAGCGACTATCAAAGACATACTCGAGTATG | Mouse |
| | | | TCTTTGATAGTCGCTCGTTTTT | |
| TRCN0000368697 | 59 | 3UTR | CCGGTATTATCTCACTCGGTATTACCTCGAGGTAA | Mouse |
| | | | TACCGAGTGAGATAATATTTTTG | |
| TRCN0000360775 | 60 | CDS | CCGGACCAATTGACAGGCCATAAAGCTCGAGCTTT | Mouse |
| | | | ATGGCCTGTCAATTGGTTTTTTG | |
| TRCN0000360846 | 61 | CDS | CCGGATTTGCCCAGCTACCTATTTCCTCGAGGAAA | Mouse |
| | | | TAGGTAGCTGGGCAAATTTTTTG | |
| TRCN0000360848 | 62 | CDS | CCGGCTAGCTGTGGATCGCCAAATTCTCGAGAATT | Mouse |
| | | | TGGCGATCCACAGCTAGTTTTTG | |
| TRCN0000002169 | 63 | CDS | CCGGCGCAGTTTAGATGTTGTTGGACTCGAGTCCA | Human |
| | | | ACAACATCTAAACTGCGTTTTT | |
| TRCN0000002170 | 64 | CDS | CCGGGTGGCTTATCATCTTATCATTCTCGAGAATG | Human |
| | | | ATAAGATGATAAGCCACTTTTT | |
| TRCN0000002171 | 65 | CDS | CCGGCCCACTGAAACGAGCAACTATCTCGAGATAG | Human |
| | | | TTGCTCGTTTCAGTGGGTTTTT | |
| TRCN0000355740 | 66 | 3UTR | CCGGGGTGTAGGTAAATCTAGTTTACTCGAGTAAA | Human |
| | | | CTAGATTTACCTACACCTTTTTG | |
| TRCN0000002172 | 67 | 3UTR | CCGGGCTGTGTTTATCGCCCAATTTCTCGAGAAAT | Human |
| | | | TGGGCGATAAACACAGCTTTTT | |
| TRCN0000002168 | 68 | CDS | CCGGGCTGTGAAAGAAGTGTGTGAACTCGAGTTCA | Human |
| | | | CACACTTCTTTCACAGCTTTTT | |
| TRCN0000194959 | 69 | CDS | CCGGCAACTTTACCTGGTTGATAACCTCGAGGTTA | Human |
| | | | TCAACCAGGTAAAGTTGTTTTTTG | |
| TRCN0000196523 | 70 | CDS | CCGGGTCATCCTCATATTATCAAACCTCGAGGTTT | Human |
| | | | GATAATATGAGGATGACTTTTTTG | |
| TRCN0000355741 | 71 | CDS | CCGGAGATAGCCGATTTCGGATTATCTCGAGATAA | Human |
| | | | TCCGAAATCGGCTATCTTTTTTG | |
| TRCN0000355739 | 72 | CDS | CCGGGAGCATGTACCTACGTTATTTCTCGAGAAAT | Human |
| | | | AACGTAGGTACATGCTCTTTTTG | |

The targeting polynucleotide can have 4 to 500 or more nucleotides. In some embodiments, the targeting polynucleotide can have 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

Also described herein are polynucleotides capable of encoding the AMPKα targeting molecule (such as a polynucleotide). As used herein, the term “encode” refers to principle that DNA can be transcribed into RNA, which can then be translated into amino acid sequences that can form proteins.

### Vectors and Vector Systems

Also provided herein are vectors and vector systems that includes one or more of the CaMKKβ and/or AMPKα targeting molecule(s) and/or polynucleotides capable of the encoding CaMKKβ and/or AMPKα targeting molecule(s) described herein. In some embodiments, the vector can contain one or more polynucleotides encoding one or more elements of one or more CaMKKβ and/or AMPKα targeting molecule(s) described herein. The vectors can be useful in producing bacterial, fungal, yeast, plant cells, animal cells, and transgenic animals that can express one or more components of the CaMKKβ and/or AMPKα targeting molecule(s) described herein. Within the scope of this disclosure are vectors containing one or more of the polynucleotide sequences described herein. The vectors and/or vector systems can be used, for example, to express one or more of the polynucleotides in a cell, such as a producer cell, to produce virus particles containing a CaMKKβ and/or AMPKα targeting molecule(s) or polynucleotide capable of encoding a CaMKKβ and/or AMPKα targeting molecule(s) described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g. a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

In some embodiments, the vector can be a bicistronic vector. In some embodiments, a bicistronic vector can be used for one or more elements of the CaMKKβ and/or AMPKα targeting molecule(s) and/or encoding polynucleotides described herein. In some embodiments, expression of elements of the CaMKKβ and/or AMPKα targeting molecule(s) or encoding polynucleotides described herein can be driven by the CBh promoter. Where the CaMKKβ targeting and/or encoding polynucleotide(s) is an RNA, its expression can be driven by a Pol III promoter, such as a U6 promoter. In some embodiments, the two are combined.

### Cell-Based Vector Amplification and Expression

Vectors can be designed for expression of one or more elements of a CaMKKβ and/or AMPKα targeting molecule(s) and/or encoding polynucleotides described herein (e.g. nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more of the CaMKKβ and/or AMPKα targeting molecule(s) and/or encoding polynucleotides so as to drive expression of the one or more CaMKKβ and/or AMPKα targeting molecule(s) and/or encoding polynucleotides described herein.

Vectors can be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some aspects, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more CaMKKβ and/or AMPKα targeting molecule(s) or encoding polynucleotides described herein are introduced into a host cell such that expression of the CaMKKβ and/or AMPKα targeting molecule(s) and/or encoding polynucleotides described herein direct formation of CaMKKβ and/or AMPKα targeting molecule(s) described herein. For example, different CaMKKβ and/or AMPKα targeting molecule(s) and/or encoding polynucleotides described herein can each be operably linked to separate regulatory elements on separate vectors. Different CaMKKβ and/or AMPKα targeting molecule(s) and/or encoding polynucleotides described herein can be delivered to an animal or mammal or cell thereof to produce an animal or mammal or cell thereof that constitutively or inducibly or conditionally expresses different CaMKKβ and/or AMPKα targeting molecule(s) and/or encoding polynucleotides described herein that incorporates one or more CaMKKβ and/or AMPKα targeting molecule(s) and/or encoding polynucleotides described herein or contains one or more cells that incorporates and/or expresses one or more CaMKKβ and/or AMPKα targeting molecule(s) and/or encoding polynucleotides described herein.

In some embodiments, two or more of the elements expressed from the same or different regulatory element(s) are combined in a single vector, with one or more additional vectors providing any components of the system not included in the first vector. CaMKKβ and/or AMPKα targeting molecule(s) and/or encoding polynucleotides described herein that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more CaMKKβ and/or AMPKα targeting molecule(s) described herein, embedded within one or more intron sequences (e.g., each in a different intron, two or more in at least one intron, or all in a single intron). In some embodiments, the CaMKKβ and/or AMPKα targeting molecule(s) or encoding polynucleotides described herein can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g. molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In aspects, the polynucleotides and/or vectors thereof described herein (such as the CaMKKβ and/or AMPKα targeting molecule(s) and/or encoding polynucleotides described herein) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g. promoters, that direct the transcription of the gene and/or translation of the encoded protein in a cell. In some embodiments, a constitutive promoter may be employed. Suitable constitutive promoters for mammalian cells are generally known in the art and include, but are not limited to SV40, CAG, CMV, EF-1α, O-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Ferl14), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the CaMKKβ and/or AMPKα targeting molecule(s) and/or encoding polynucleotides described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the CaMKKβ and/or AMPKα targeting molecule(s) and/or encoding polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the CaMKKβ and/or AMPKα targeting molecule(s) and/or encoding polynucleotides are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more CaMKKβ and/or AMPKα targeting molecule(s) and/or encoding polynucleotides described herein, a light-responsive cytochrome heterodimer (e.g. from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g. aspects of inducible DNA binding proteins and methods of use and can be adapted for use with the present invention.

In some embodiments, transient or inducible expression can be achieved by including, for example, chemical-regulated promotors, i.e. whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing an CaMKKβ and/or AMPKα targeting molecule(s) and/or encoding polynucleotides to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the CaMKKβ and/or AMPKα targeting molecule(s) (e.g. such as a polynucleotide) and/or encoding polynucleotides can be operably linked, fused to, or otherwise modified to include a polynucleotide that encodes or is a selectable marker or tag, which can be a polynucleotide or polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more CaMKKβ and/or AMPKU targeting molecule(s) and/or encoding polynucleotides described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more CaMKKβ and/or AMPKα targeting molecule(s) and/or encoding polynucleotides described herein via suitable linker, such as a glycine or glycine serine linkers as short as GS or GG up to (GGGGG)₃ (SEQ ID NO: 73) or (GGGGS)₃ (SEQ ID NO: 74). Other suitable linkers are generally known in the art and/or are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the CaMKKβ and/or AMPKα targeting molecule(s) and/or encoding polynucleotides and/or products expressed therefrom include the targeting moiety and can be targeted to specific cells, tissues, organs, etc. In some embodiments, such as non-viral carriers, the targeting moiety can be attached to the carrier (e.g. polymer, lipid, inorganic molecule etc.) and can be capable of targeting the carrier and any attached or associated CaMKKβ and/or AMPKα targeting molecule(s) and/or encoding polynucleotides to specific cells, tissues, organs, etc.

### Cell-Free Vector and Polynucleotide Expression

In some embodiments, the CaMKKβ and/or AMPKα targeting molecule(s) and/or encoding polynucleotides can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g. translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g. 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g. reticulocyte lysates and wheat germ extracts). Some translation systems can utilize a DNA template as a starting material (e.g. E. coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more aspects of the vectors or vector systems described herein can be codon optimized. In some embodiments, one or more polynucleotides contained in a vector (“vector polynucleotides”) described herein that are in addition to an optionally codon optimized polynucleotide encoding CaMKKβ and/or AMPKα targeting molecule(s) described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gown, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some aspects, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an CaMKKβ and/or AMPKU targeting molecule(s) and/or encoding polynucleotide of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as a CaMKKβ and/or AMPKα targeting molecule(s) and/or encoding polynucleotides of the present invention.

### Naked Polynucleotides

In some embodiments, one or more CaMKKβ and/or AMPKα targeting molecule(s) and/or encoding polynucleotides described elsewhere herein can be included in a naked polynucleotide. The term of art “naked polynucleotide” as used herein refers to polynucleotides that are not associated with another molecule (e.g. proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the CaMKKβ and/or AMPKα targeting molecule(s) and/or encoding polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g. plasmids and artificial chromosomes), molecules that contain portions that are single stranded and portions that are double stranded (e.g. ribozymes), and the like. In some embodiments, the naked polynucleotide contains only the CaMKKβ and/or AMPKα targeting molecule(s) and/or encoding polynucleotides of the present invention. In some embodiments, the naked polynucleotide can contain other nucleic acids and/or polynucleotides in addition to the CaMKKβ and/or AMPKα targeting molecule(s) and/or encoding polynucleotides of the present invention. The naked polynucleotides can include one or more elements of a transposon system. Transposons and system thereof are described in greater detail elsewhere herein.

### Non-Viral Polynucleotide Vectors

In some embodiments, one or more of the CaMKKβ and/or AMPKα targeting molecule(s) and/or encoding polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g. Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g. Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In aspects, the S/MAR sequence is located downstream of an actively transcribed polynucleotide (e.g. one or more CaMKKβ and/or AMPKα targeting molecule(s) and/or encoding polynucleotides of the present invention) included in the non-viral polynucleotide vector. In some embodiments, the S/MAR can be a S/MAR from the beta-interferon gene cluster. See e.g. Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these aspects, the transposon vector lacks one or more polynucleotide sequences encoding proteins required for transposition. In some embodiments, the non-autonomous transposon vectors lack one or more Ac elements.

In some aspects a non-viral polynucleotide transposon vector system can include a first polynucleotide vector that contains the CaMKKβ and/or AMPKα targeting molecule(s) and/or encoding polynucleotides of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g. the CaMKKβ and/or AMPKα targeting molecule(s) and/or encoding polynucleotides of the present invention) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g. one or more of the CaMKKβ and/or AMPKα targeting molecule(s) and/or encoding polynucleotides of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, Sleeping Beauty transposon system (Tcl/mariner superfamily) (see e.g. Ivics et al. 1997. Cell. 91(4): 501-510), piggyBac (piggyBac superfamily) (see e.g. Li et al. 2013 110(25): E2279-E2287 and Yusa et al. 2011. PNAS. 108(4): 1531-1536), Tol2 (superfamily hAT), Frog Prince (Tcl/mariner superfamily) (see e.g. Miskey et al. 2003 Nucleic Acid Res. 31(23):6873-6881) and variants thereof.

### Chemical Carriers

In some embodiments, the CaMKKβ and/or AMPKα targeting molecule(s) and/or encoding polynucleotides can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the CaMKKβ and/or AMPKα targeting molecule(s) and/or encoding polynucleotides of the present invention), (2) those capable of targeting specific cells, (3) those capable of increasing delivery of the polynucleotide (such as the CaMKKβ and/or AMPKα targeting molecule(s) and/or encoding polynucleotides of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the CaMKKβ and/or AMPKα targeting molecule(s) and/or encoding polynucleotides described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some aspects, the inorganic particles can be optimized to protect an entrapped molecule from degradation, the Suitable inorganic particles that can be used as non-viral carriers in this context can include, but are not limited to, calcium phosphate, silica, metals (e.g. gold, platinum, silver, palladium, rhodium, osmium, iridium, ruthenium, mercury, copper, rhenium, titanium, niobium, tantalum, and combinations thereof), magnetic compounds, particles, and materials, (e.g. supermagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g. such as a CaMKKβ and/or AMPKα targeting molecule(s) and/or encoding polynucleotide of the present invention). In some embodiments, chemical non-viral carrier systems can include a polynucleotide such as the CaMKKβ and/or AMPKα targeting molecule(s) and/or encoding polynucleotides of the present invention) and a lipid (such as a cationic lipid). These are also referred to in the art as lipoplexes. Other aspects of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g. the CaMKKβ and/or AMPKα targeting molecule(s) and/or encoding polynucleotides of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g. polymer-based carriers and lipid-based carriers to functionalize these carriers). In some embodiments, the functionalization is targeting a host cell. Suitable polymers that can be included in the polymer-based non-viral carrier can include, but are not limited to, polyethyleneimine (PEI), chitosan, poly (DL-lactide) (PLA), poly (DL-Lactide-co-glycoside) (PLGA), dendrimers (see e.g. US Pat. Pub. 2017/0079916 whose techniques and compositions can be adapted for use with the CaMKKβ and/or AMPKU targeting molecule(s) and/or encoding polynucleotides of the present invention), polymethacrylate, and combinations thereof.

In some aspects, the non-viral carrier can be configured to release a CaMKKβ and/or AMPKα targeting molecule(s) and/or encoding polynucleotides that is associated with or attached to the non-viral carrier in response to an external stimulus, such as pH, temperature, osmolarity, concentration of a specific molecule or composition (e.g. calcium, NaCl, and the like), pressure and the like. In some embodiments, the non-viral carrier can be a particle that is configured includes one or more of the CaMKKβ and/or AMPKα targeting molecule(s) and/or encoding polynucleotides describe herein and an environmental triggering agent response element, and optionally a triggering agent. In some embodiments, the particle can include a polymer that can be selected from the group of polymethacrylates and polyacrylates. In some embodiments, the non-viral particle can include one or more aspects of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the CaMKKβ and/or AMPKα targeting molecule(s) and/or encoding polynucleotides of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a CaMKKβ and/or AMPKα targeting molecule(s) and/or encoding polynucleotide of the present invention, into a virus particle and producing said virus particle when used alone or with one or more other viral vectors (such as in a viral vector system). Viral vectors and systems thereof can be used for producing viral particles for delivery of and/or expression of one or more CaMKKβ and/or AMPKα targeting molecule(s) and/or encoding polynucleotides described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other aspects of viral vectors and viral particles produce therefrom are described elsewhere herein. In some embodiments, the viral vectors are configured to produce replication incompetent viral particles for improved safety of these systems.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the CaMKKβ and/or AMPKα targeting molecule(s) and/or encoding polynucleotides can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the CaMKKβ and/or AMPKα targeting molecule(s) and/or encoding polynucleotides described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g. VSV-G) and other accessory genes (e.g. vif, vprm vpu, nef, and combinations thereof), regulatory genes (e.g. tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g. vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g. VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In some aspects, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In aspects, the third-generation lentiviral vector system can include at least two packaging vectors, with the gag-pol being present on a different vector than the rev gene.

In some aspects, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the CaMKKβ targeting or encoding polynucleotides of the present invention.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g. Hanawa et al. Molec. Ther. 2002 5(3) 242-251), modified Sindbis virus envelope proteins (see e.g. Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g. Girard-Gagnepain et al. 2014. Blood. 124: 1221-1231); Tupaia paramyxovirus glycoproteins (see e.g. Enkirch T. et al., 2013. Gene Ther. 20:16-23); measles virus glycoproteins (see e.g. Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g. Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g. Chamoun-Emaneulli et al. 2015. Biotechnol. Bioeng. 112:2611-2617, Ramirez et al. 2013. Protein. Eng. Des. Sel. 26:215-233. In these aspects, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g. Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g. a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver a CaMKKβ and/or AMPKα targeting molecule(s) and/or encoding polynucleotides described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g. antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

### Adenoviral Vectors, Helper-dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g. Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments, the vector can be a helper-dependent adenoviral vector or system thereof. These are also referred to in the art as “gutless” or “gutted” vectors and are a modified generation of adenoviral vectors (see e.g. Thrasher et al. 2006. Nature. 443:E5-7). In aspects of the helper-dependent adenoviral vector system one vector (the helper) can contain all the viral genes required for replication but contains a conditional gene defect in the packaging domain. The second vector of the system can contain only the ends of the viral genome, one or more CaMKKβ and/or AMPKα targeting molecule(s) and/or encoding polynucleotides, and the native packaging recognition signal, which can allow selective packaged release from the cells (see e.g. Cideciyan et al. 2009. N Engl J Med. 361:725-727). Helper-dependent adenoviral vector systems have been successful for gene delivery in several contexts (see e.g. Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the CaMKKβ and/or AMPKα targeting molecule(s) and/or encoding polynucleotides described herein. In some embodiments, the polynucleotide to be delivered via the viral particle produced from a helper-dependent adenoviral vector or system thereof can be up to about 37 kb. Thus, in some embodiments, a adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g. Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use with the CaMKKβ and/or AMPKα targeting molecule(s) and/or encoding polynucleotides of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g. Ehrhardt et al. 2007. Mol. Ther. 15:146-156 and Liu et al. 2007. Mol. Ther. 15:1834-1841, whose techniques and vectors described therein can be modified and adapted for use with the CaMKKβ and/or AMPKα targeting molecule(s) and/or encoding polynucleotides of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g. Ehrhardt et al. 2007. Mol. Ther. 156:146-156 and Shuji et al. 2011. Mol. Ther. 19:76-82, whose techniques and vectors described therein can be modified and adapted for use with the CaMKKβ and/or AMPKα targeting molecule(s) and/or encoding polynucleotides described herein.

### Adeno Associated Viral (AAV) Vectors

In an embodiment, the vector can be an adeno-associated virus (AAV) vector. See e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008) and is recapitulated below in Table 3.

TABLE 3

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g. the CaMKKβ and/or AMPKα targeting molecule(s) and/or encoding polynucleotide(s)).

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g. 2009. Trobridge. Exp. Opin. Biol. Ther. 9:1427-1436, whose techniques and vectors described therein can be modified and adapted for use with the CaMKKβ targeting or encoding polynucleotides of the present invention. In some embodiments, where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments, the CaMKKβ and/or AMPKα targeting molecule(s) and/or encoding polynucleotides included in the HSV-based viral vector or system thereof can sum from about 0.001 to about 150 kb. HSV-based vectors and systems thereof have been successfully used in several contexts including various models of neurologic disorders. See e.g. Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the CaMKKβ and/or AMPKα targeting molecule(s) and/or encoding polynucleotides described herein.

### Poxvirus Vectors

In some embodiments, the vector can be a poxvirus vector or system thereof. In some embodiments, the poxvirus vector can result in cytoplasmic expression of one or more CaMKKβ and/or AMPKα targeting molecule(s) and/or encoding polynucleotides described herein. In some embodiments, the capacity of a poxvirus vector or system thereof can be about 25 kb or more. In some embodiments, a poxivirus vector or system thereof can include a CaMKKβ and/or AMPKα targeting molecule(s) and/or encoding polynucleotides described herein.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more CaMKKβ and/or AMPKα targeting molecule(s) and/or encoding polynucleotides described herein are as used in the foregoing documents, such as WO 2014/093622 (PCT/US2013/074667) and are discussed in greater detail herein.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g. pol, gag, and/or VSV-G) and/or other supporting genes.

In some aspects, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g. an CaMKKβ and/or AMPKα targeting molecule(s) and/or encoding polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g. the CaMKKβ and/or AMPKα targeting molecule(s) and/or encoding polynucleotide(s)). In some aspects, a method of producing AAV particles from AAV vectors and systems thereof can be a “helper free” method, which includes co-transfection of an appropriate producing cell line with three vectors (e.g. plasmid vectors): (1) an AAV vector that contains a polynucleotide of interest (e.g. the CaMKKβ and/or AMPKα targeting molecule(s) and/or encoding polynucleotide(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., CaMKKβ and/or AMPKα targeting molecule(s) and/or encoding polynucleotides, proteins, enzymes, mutant forms thereof, fusion proteins thereof, etc.), and virus particles (such as from viral vectors and systems thereof).

One or more CaMKKβ and/or AMPKα targeting molecule(s) and/or encoding polynucleotides can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g. injections), ballistic polynucleotides (e.g. particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of CaMKKβ and/or AMPKα targeting molecule(s) and/or encoding polynucleotides or vector(s) described herein to cells via particles. The term “particle” as used herein, refers to any suitable sized particles for delivery of the CaMKKβ and/or AMPKU targeting molecule(s) and/or encoding polynucleotides and/or vector(s) described herein. Suitable sizes include macro-, micro-, and nano-sized particles. In some embodiments, any of the of the CaMKKβ and/or AMPKα targeting molecule(s) and/or encoding polynucleotides, vector(s) and combinations thereof described herein can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in aspects, particle delivery can also be advantageous for other CaMKKβ and/or AMPKU targeting molecule(s) and/or encoding polynucleotides and formulations described elsewhere herein.

### Cells

Also described herein are various embodiments of cells that contain one or more CaMKKβ and/or AMPKα targeting molecule(s) and/or encoding polynucleotides, vectors, and/or vector systems described herein and methods of delivering one or more CaMKKβ and/or AMPKα targeting molecule(s) and/or encoding polynucleotides, vectors, and/or vector systems described herein to one or more cells. Delivery can be in vitro, ex vivo, or in vivo. In some embodiments, the cells are eukaryotic cells. In some embodiments, the cells are prokaryotic cells. The cell can be a mammalian cell, yeast cell, bacterial cell, fungal cell, or a plant cell. In some embodiments, the cell is an auditory neuron, hair cell (e.g. an outer hair cell), or other otic cell or neuron. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some embodiments, the cells can be or form a disease model, such as a hearing loss disease model. In some methods, the disease model can be used to study the effects of mutations on the animal or cell and development and/or progression of the disease using measures commonly used in the study of the disease. In some other embodiments, such a disease model is useful for studying the effect of a pharmaceutically active compound on the disease, such as the CaMKKβ and/or AMPKα targeting molecule(s) and/or encoding polynucleotides of the present invention.

In some embodiments, the cells are capable of packaging one or more CaMKKβ and/or AMPKα targeting molecule(s) and/or encoding polynucleotides into virus particles. These cells are also referred to herein as producer cells. In some embodiments, the CaMKKβ and/or AMPKα targeting molecule(s) and/or encoding polynucleotide(s) can be delivered to a cell via a viral particle. As previously described, the viral particle containing a packaged CaMKKβ and/or AMPKα targeting molecule(s) and/or encoding polynucleotide(s) can be generated and harvested from a producer cell. Producer cells can express one or more viral vectors or vector systems described elsewhere herein. In some embodiments, viral particle delivery can be via a single dose or include one or more booster doses. In some embodiments the doses can be or contain at least 1×10⁵ particles (also referred to as particle units, pu) of a viral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁰ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. In other embodiments, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of a viral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of a viral vector. In some embodiments, the delivery to the cell is via an AAV.

In some embodiments, the dosage of the AAV is for in vivo delivery to a human. In some embodiments, the dosage can contain about 0.5 to 5 mL of a saline solution that can contain about 1×10¹⁰ AAV/ml solution. The dosage can be balanced to adjust the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

The CaMKKβ and/or AMPKα targeting molecule(s) and/or encoding polynucleotides, vectors, and/or vector systems described herein can also be delivered to a cell using non-viral delivery of the CaMKKβ and/or AMPKα targeting molecule(s) and/or encoding polynucleotides, vectors, and/or vector systems described herein. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target cells and/or tissues (e.g. in vivo administration).

Cells to which the CaMKKβ and/or AMPKα targeting molecule(s) and/or encoding polynucleotides, vectors, and/or vector systems described herein are delivered can also be referred to herein as host cells. In some embodiments, a host cell is transiently or non-transiently transfected with one or more vectors described herein. In some embodiments, a cell is transfected as it naturally occurs in a subject optionally to be reintroduced therein. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, ClR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-IOT1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/ARI, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences.

In some embodiments the RNA molecules of the invention are delivered in liposome or lipofectin formulations and the like and can be prepared by methods well known to those skilled in the art. Such methods are described, for example, in U.S. Pat. Nos. 5,593,972, 5,589,466, and 5,580,859, which are herein incorporated by reference. Delivery systems aimed specifically at the enhanced and improved delivery of siRNA into mammalian cells have been developed, (see, for example, Shen et al FEBS Let. 2003, 539:111-114; Xia et al., Nat. Biotech. 2002, 20:1006-1010; Reich et al., Mol. Vision. 2003, 9: 210-216; Sorensen et al., J. Mol. Biol. 2003, 327: 761-766; Lewis et al., Nat. Gen. 2002, 32: 107-108 and Simeoni et al., NAR 2003, 31, 11: 2717-2724) and may be applied to the present invention. siRNA has recently been successfully used for inhibition of gene expression in primates (see for example. Tolentino et al., Retina 24(4):660 which may also be applied to the present invention. In some embodiments, the liposome can be a stable nucleic-acid-lipid particle (SNALP) (see, e.g., Morrissey et al., Nature Biotechnology, Vol. 23, No. 8, August 2005).

Means of delivery of RNA also preferred include delivery of RNA via particles (Cho, S., Goldberg, M., Son, S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 3112-3118, 2010) or exosomes (Schroeder, A., Levins, C., Cortez, C., Langer, R., and Anderson, D., Lipid-based nanotherapeutics for siRNA delivery, Journal of Internal Medicine, 267: 9-21, 2010, PMID: 20059641). In some embodiments, the particle can be a nanoparticle. In some embodiments, the nanoparticle is a dendrimer nanoparticle, which can be optionally modified (see e.g. US 20170079916). In some embodiments, the nanoparticles can be configured to their payload when exposed to acidic conditions, wherein the microparticles comprise at least one agent to be delivered, a pH triggering agent, and a polymer, wherein the polymer is selected from the group of polymethacrylates and polyacrylates (see e.g. US US20050123596). In some embodiments, the particles can be lipid-protein-sugar particles, where the polynucleotide is encapsulated in a lipid-protein-sugar matrix by contacting the polynucleotide with a lipid, a protein, and a sugar; and spray drying mixture of the polynucleotide, the lipid, the protein, and the sugar to make microparticles (see e.g. US 20020150626).

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

A particle in accordance with the present invention is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (e.g. the polynucleotides, vectors, and vector systems described herein).

In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, concerning particles, methods of making and using them and measurements thereof.

In some embodiments, the particle is a lipid nanoparticle (LNP). LNP compositions and methods of preparing and LNPs are generally known in the art. See e.g. Coelho et al., N Engl J Med 2013; 369:819-29, Tabernero et al., Cancer Discovery, April 2013, Vol. 3, No. 4, pages 363-470, Rosin et al, Molecular Therapy, vol. 19, no. 12, pages 1286-2200, December 2011, Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014).

Self-assembling nanoparticles with RNA (such as those of the present invention) can be constructed with polyethyleneimine (PEI) that is PEGylated with an Arg-Gly-Asp (RGD) peptide ligand attached at the distal end of the polyethylene glycol (PEG) and used to deliver the RNA to a cell (see, e.g., Schiffelers et al., Nucleic Acids Research, 2004, Vol. 32, No. 19).

Nanoplexes can be used to deliver the polynucleotides described herein to a cell. See e.g. PNAS, Sep. 25, 2007, vol. 104, no. 39, which can be adapted for sue with the present invention.

In some embodiments, cell penetrating peptides can be used to deliver the polynucleotides, vectors, or vector system of the present invention to a cell. CPPs are short peptides that facilitate cellular uptake of various molecular cargo (from nanosize particles to small chemical molecules and large fragments of DNA). See e.g. U.S. Pat. Nos. 8,372,951, 8,575,305; 8,614,194 and 8,044,019, which can be adapted for use with the present invention.

Other techniques and compositions useful for delivering the polynucleotides, vectors, and vector systems of the present invention will be appreciated by those of ordinary skill in the art in view of this disclosure.

### Pharmaceutical Formulations

Also described herein are pharmaceutical formulations that can contain an amount, effective amount, and/or least effective amount, of one or more CaMKKβ and/or AMPKU targeting molecule(s) and/or encoding polynucleotides, vectors, vector systems, cells, or a combination thereof as described in greater detail elsewhere herein a pharmaceutically acceptable carrier. In some embodiments, the compound can be present in the pharmaceutical formulation as a pharmaceutically acceptable salt.

The compounds described herein can be provided to a subject in need thereof as an ingredient, such as an active ingredient, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered via any suitable method to a subject in need thereof. In some embodiments, the subject in need thereof has or is susceptible to or will be exposed to an acoustic and/or chemical insult. The acoustic or chemical insult can be acute or chronic. The acoustic insult can be noise that, without treatment or prevention, such as the polynucleotides, vectors, cells, and formulations thereof described herein, is such that it can cause noise-induced-hearing loss, damage and/or death to one or more hair cells (such as outer hair cells), damage and/or death of cells of the stria vascularis, and/or auditory nerve cell and/or spiral ganglion neuron damage and/or death (also referred to as excitotoxicity). In some embodiments, the acoustic insult is a noise that is or is greater than about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 dB.

In some embodiments, the chemical insult is an ototoxic agent that is delivered to the subject. There are many agents, including prescription and non-prescription, pharmaceutical agents as well as environmental agents that are known to be ototoxic. These generally include some antibiotic, loop diuretics, chemotherapeutic agents, antiseptics and disinfectants, and others. Exemplary ototoxic agents, include but are not limited to aminoglycosides (e.g. gentamicin and tobramycin), macrolides (e.g. erythromycin), furosemide, ethacrynic acid, bumetanide, Pt-containing chemotherapeutic agents (e.g. cisplatin, carboplatin, and oxaliplatin), vinca alkaloids (e.g. vincristine), chlorhexidine, ethyl alcohol, acetic acid, propylene glycol, and quaternary ammonium compounds (particularly if they enter the inner ear), quinine, aspirin, other siacylates, erectile dysfunction medications (e.g. Viagra), pesticides, solvents (e.g. toluene, styrene, and xylene), asphyxiants, and heavy metals, carbon monoxide and combinations thereof.

### Pharmaceutically Acceptable Carriers and Auxiliary Ingredients and Agents

The pharmaceutical formulations containing an amount, such as an effective amount, least effective amount, and/or pharmaceutically effective amount of a CaMKKβ and/or AMPKα targeting molecule(s) and/or encoding polynucleotides, vectors, vector systems, cells, or a combination thereof described herein or a derivative thereof can further include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In addition to the amount of CaMKKβ and/or AMPKα targeting molecule(s) and/or encoding polynucleotides, vectors, vector systems, cells, or a combination thereof the pharmaceutical formulations can also include an effective amount of auxiliary active agents, including but not limited to, antisense or RNA interference molecules, anti-infectives, chemotherapeutics, or antineoplastic agents, hormones, antibiotics, antivirals, immunomodulating agents, antinausea, pain modifying compounds (such as opiates), anti-inflammatory agents, antipyretics, antibiotics, and/or antibodies or fragments thereof. In some embodiments, the auxiliary active agent is an ototoxic agent, which are previously discussed herein.

Effective Amounts of the CaMKKβ and/or AMPKα Targeting Compositions and Auxiliary Active Agents

In some embodiments, the effective amount, least effective amount, and/or pharmaceutically effective amount of the CaMKKβ and/or AMPKα targeting molecule(s) and/or encoding polynucleotides, vectors, vector systems, cells, or a combination thereof can be effective to treat or prevent hearing loss, (e.g. acquired hearing loss as a result of an acoustic and/or chemical insult), treat or prevent hair cell damage or death (e.g. outer hair cell damage or death), auditory nerve cell damage or death, stria vascularis cell damage or death, spiral ganglion neuron damage and/or death (also referred to as excitotoxicity), or a combination thereof in the subject. The effective amount, least effective amount, and/or pharmaceutically effective amount of the CaMKKβ and/or AMPKα targeting molecule(s) and/or encoding polynucleotides, vectors, vector systems, cells, or a combination thereof described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges. In some embodiments, the effective concentration of the CaMKKβ and/or AMPKα targeting molecule(s) and/or encoding polynucleotides, vectors, vector systems, cells, or a combination thereof can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In further embodiments, the effective of the CaMKKβ and/or AMPKα targeting molecule(s) and/or encoding polynucleotides, vectors, vector systems, cells, or a combination thereof amount can range from 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pL, nL, μL, mL, or L or be any numerical value with any of these ranges.

In yet other embodiments, the effective amount of the CaMKKβ and/or AMPKU targeting molecule(s) and/or encoding polynucleotides, vectors, vector systems, cells, or a combination thereof can range from about 0, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

In embodiments where there is an auxiliary active agent contained in the pharmaceutical formulation, the effective amount of the auxiliary active agent will vary depending on the auxiliary active agent. Auxiliary active agents are described in greater detail elsewhere herein. In some embodiments, the auxiliary active agent can be an ototoxic agent.

In some embodiments, the effective amount of the auxiliary active agent can range from 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges. In other embodiments, the effective amount of the auxiliary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges. In further embodiments, the effective amount of the auxiliary active agent can range from 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pL, nL, μL, mL, or L or be any numerical value with any of these ranges. In some embodiments, the concentration of the auxiliary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges. In yet other embodiments, the effective amount of the auxiliary active agent can range from about 0, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

The auxiliary active agent can be included in the pharmaceutical formulation or can exist as a stand-alone compound or pharmaceutical formulation that can be administered contemporaneously or sequentially with the compound, derivative thereof, or pharmaceutical formulation thereof. In embodiments where the auxiliary active agent is a stand-alone compound or pharmaceutical formulation, the effective amount of the auxiliary active agent can vary depending on the auxiliary active agent used.

### Dosage Forms

In some embodiments, the pharmaceutical formulations described herein can be in a dosage form. The dosage form can be administered to a subject in need thereof. The dosage form can be effective generate specific concentration, such as an effective concentration, at a given site in the subject in need thereof. In some cases, the dosage form contains a greater amount of the active ingredient than the final intended amount needed to reach a specific region or location within the subject to account for loss of the active components such as via first and second pass metabolism.

The dosage forms can be adapted for administration by any appropriate route. Appropriate routes include, but are not limited to, otic (e.g. via contact or placement on the round window membrane or via a posterior semi-circular canal method), oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for otic administration can be discrete dosage units such as powders, solutions, gels, or suspensions in aqueous or non-aqueous liquids, foams, oil-in-water liquid emulsions, water-in-oil liquid emulsions. Dosage forms adapted for otic administration can be delivered as a powder, gel, foam, spray, or liquid solution. Other appropriate dosage forms are discussed with respect to topical dosage forms. Otic dosage forms can be adapted for delivery to the inner ear via placement or contact with the round window membrane of an ear. Otic dosage forms can be adapted for delivery to the inner ear via a posterior semi-circular canal method. Exemplary formulations for round window membrane delivery of a compound or cell described herein is discussed in e.g. Oishi et al., 2013. J Neurosci. 32:12421-12430, which can be adapted for use with the present invention. Gou et al., 2018. J Vis. Exp. describes an example of drug delivery to the inner ear, which can be adapted in view of the present description for delivery of a compound or cell described herein to the inner ear. Dosage forms suitable for intratympanic delivery, intracochlear delivery, semicircular canal delivery, are also described in, e.g., Swan et al., 2008. Adv Drug Deliv Rev. 60(15): 1583-1599; Rybak et al., 2019. Front Cell Neurosci. https://doi.org/10.3389/fncel.2019.00300; Liu et al. 2018. Drug Dev and Industr. Pharmacy. 44(9): 1395-1408; and Isgrig and Chien. 2018. J Vis Exp. 133:56648 doi: 10.3791/56648.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution.

The oral dosage form can be administered to a subject in need thereof.

Where appropriate, the dosage forms described herein can be microencapsulated.

The dosage form can also be prepared to prolong or sustain the release of any ingredient. In some embodiments, a compound described herein (e.g. a CaMKKβ and/or AMPKα targeting molecule(s) and/or encoding polynucleotides, vectors, vector systems, cells, or a combination thereof), auxiliary active ingredient, and/or pharmaceutically acceptable salt thereof can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, compound, derivative thereof, auxiliary active ingredient, and/or pharmaceutically acceptable salt thereof are incorporated into a liposome. In some embodiments, a compound described herein (e.g. a CaMKKβ and/or AMPKα targeting molecule(s) and/or encoding polynucleotides, vectors, vector systems, cells, or a combination thereof), auxiliary active ingredient, and/or pharmaceutically acceptable salt thereof can be integrated into the lipid membrane of the liposome. In other embodiments, a compound described herein (e.g. a CaMKKβ and/or AMPKα targeting molecule(s) and/or encoding polynucleotides, vectors, vector systems, cells, or a combination thereof), derivative thereof, auxiliary active ingredient, and/or pharmaceutically acceptable salt thereof are contained in the aqueous phase of the liposome. In embodiments where the dosage form is a liposome, the pharmaceutical formulation is thus a liposomal formulation.

The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a compound described herein (e.g. CaMKKβ and/or AMPKU targeting molecule(s) and/or encoding polynucleotides, vectors, vector systems, cells, or a combination thereof), auxiliary active ingredient, and/or pharmaceutically acceptable salt thereof can be formulated with a paraffinic or water-miscible ointment base. In other embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the compound, derivative thereof, auxiliary active ingredient, and/or pharmaceutically acceptable salt thereof in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient, which may be generated by various types of metered dose pressurized aerosols, nebulizers, or insufflators.

The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a compound described herein (e.g. a CaMKKβ and/or AMPKU targeting molecule(s) and/or encoding polynucleotides, vectors, vector systems, cells, or a combination thereof) or a structural analogue thereof, auxiliary active ingredient, and/or pharmaceutically acceptable salt thereof and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a compound, derivative thereof, auxiliary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time. The aerosol formulations can be administered to a subject in need thereof.

For some dosage forms suitable and/or adapted for inhaled administration, the pharmaceutical formulation is a dry powder inhalable-formulation. In addition to the compound described herein (e.g. a CaMKKβ and/or AMPKα targeting molecule(s) and/or encoding polynucleotides, vectors, vector systems, cells, or a combination thereof), auxiliary active ingredient, and/or pharmaceutically acceptable salt thereof, such a dosage form can contain a powder base such as lactose, glucose, trehalose, mannitol, and/or starch. In some of these embodiments, the compound, derivative thereof, auxiliary active ingredient, and/or pharmaceutically acceptable salt thereof is in a particle-size reduced form. In further embodiments, a performance modifier, such as L-leucine or another amino acid, cellobiose oxoacetate, and/or metals salts of stearic acid, such as magnesium or calcium stearate.

In some embodiments, the aerosol formulations are arranged so that each metered dose of aerosol contains a predetermined amount of an active ingredient, such as the one or more of the compounds described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriocytes, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets.

The parenteral formulations can be administered to a subject in need thereof.

For some embodiments, the dosage form contains a predetermined amount of a compound described herein (e.g. a CaMKKβ and/or AMPKα targeting molecule(s) and/or encoding polynucleotides, vectors, vector systems, cells, or a combination thereof) or a structural analogue thereof, auxiliary active ingredient, and/or pharmaceutically acceptable salt thereof per unit dose. In an embodiment, the predetermined amount of the compound described herein (e.g. a CaMKKβ and/or AMPKα targeting molecule(s) and/or encoding polynucleotides, vectors, vector systems, cells, or a combination thereof), auxiliary active ingredient, and/or pharmaceutically acceptable salt thereof can be an effective amount, a least effect amount, and/or a pharmaceutically effective amount.

In other embodiments, the predetermined amount of the compound and/or derivative thereof can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

### Kits

The pharmaceutical formulations provided herein can be presented as a combination kit. As used herein, the terms “combination kit” or “kit of parts” refers to the compounds, or pharmaceutical formulations and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

When the agents are not administered simultaneously, the combination kit can contain each agent in separate pharmaceutical formulations. The separate pharmaceutical formulations can be contained in a single package or in separate packages within the kit.

In some embodiments, the combination kit also includes instructions printed on or otherwise contained in a tangible medium of expression. The instructions can provide information regarding the content of the compound or pharmaceutical formulations contained therein, safety information regarding the content of the compound(s) or pharmaceutical formulation(s) contained therein, information regarding the dosages, indications for use, and/or recommended treatment regimen(s) for the compound(s) and/or pharmaceutical formulations contained therein. In some embodiments, the instructions can provide directions for administering the compositions, compounds, pharmaceutical formulations described herein to a subject in need thereof. In some embodiments, the subject in need thereof can in need of a treatment or prevention for hearing loss, (e.g. acquired hearing loss as a result of an acoustic and/or chemical insult), hair cell damage or death (e.g. outer hair cell damage or death), auditory nerve cell damage or death, stria vascularis cell damage or death, spiral ganglion neuron damage and/or death (also referred to as excitotoxicity), or a combination thereof. In some embodiments, the subject in need thereof can have received an ototoxic agent. In some embodiments, the subject in need thereof can be currently receiving an ototoxic agent. In some embodiments, the subject in need thereof will be receiving an ototoxic agent within about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more minutes, hours, days, or months from the time of administration of a compound of the CaMKKβ and/or AMPKα targeting molecule(s) and/or encoding polynucleotides, vectors, vector systems, cells, or a combination thereof of the present invention. In some embodiments, the subject in need thereof has been or will be exposed to an acute or chronic acoustic insult.

### Methods of Use

Any amount of the compounds (e.g. the CaMKKβ and/or AMPKα targeting molecule(s) and/or encoding polynucleotides, vectors, vector systems, cells, pharmaceutical formulations, or a combination thereof described herein can be administered to a subject in need thereof one or more times per day, week, month, or year. In some embodiments, the pharmaceutical formulation administered contains an effective amount, a least effective amount, amount and/or a pharmaceutically effective amount of the CaMKKβ and/or AMPKU targeting molecule(s) and/or encoding polynucleotides, vectors, vector systems, cells, or a combination thereof of the present invention. For example, the pharmaceutical formulations can be administered in a daily dose. This amount may be given in a single dose per day. In other embodiments, the daily dose may be administered over multiple doses per day, in which each containing a fraction of the total daily dose to be administered (sub-doses). In some embodiments, the number of doses delivered per day is 2, 3, 4, 5, or 6. In further embodiments, the compounds, formulations, or salts thereof are administered one or more times per week, such as 1, 2, 3, 4, 5, or 6 times per week. In other embodiments, the compounds, formulations, or salts thereof are administered one or more times per month, such as 1 to 5 times per month. In still further embodiments, the compounds, formulations, or salts thereof are administered one or more times per year, such as 1 to 11 times per year.

In some embodiments, a method of treating or preventing hearing loss in a subject can include administering an amount of a CaMKKβ and/or AMPKα targeting molecule(s) and/or encoding polynucleotide, vector, vector systems, cells, a pharmaceutical formulation thereof, or a combination thereof of the present invention, or a combination thereof to the subject.

In some a method of treating or preventing hair cell (e.g. outer hair cell) damage or death in a subject can include administering an amount of a CaMKKβ and/or AMPKα targeting molecule(s) and/or encoding polynucleotide, vector, vector systems, cells, a pharmaceutical formulation thereof, or a combination thereof, to the subject.

In some a method of treating or preventing auditory nerve cell damage and/or death in a subject can include administering an amount of a CaMKKβ and/or AMPKα targeting molecule(s) or encoding polynucleotide, vector, vector systems, cells, a pharmaceutical formulation thereof, or a combination thereof to the subject.

In some a method of treating or preventing stria vascularis cell damage and/or death in a subject can include administering an amount of a CaMKKβ and/or AMPKα targeting molecule(s) or encoding polynucleotide, vector, vector systems, cells, a pharmaceutical formulation thereof, or a combination thereof to the subject.

In some a method of treating or preventing spiral ganglion neuron damage and/or death in a subject can include administering an amount of a CaMKKβ and/or AMPKα targeting molecule(s) or encoding polynucleotide, vector, vector systems, cells, a pharmaceutical formulation thereof, or a combination thereof to the subject.

In some embodiments, administering occurs before, during, and/or after an acoustic and/or chemical insult to the ear of a subject. In some embodiments, the acoustic and/or chemical insult is an ototoxic agent. In some embodiments, administering occurs 1-60, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days prior to the subject being exposed to an acoustic and/or chemical insult. In some embodiments, administering occurs 1-120 or more days after an acoustic and/or chemical insult to the ear of a subject, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 days post insult.

In some embodiments, a method of treating or preventing an acquired (i) hearing loss, (ii) ototoxicity, (iii) outer hair cell damage or death, (iv) loss of an outer hair cell function, or a combination thereof can include
a. administering an amount of an AMPK alpha targeting RNAi molecule to the subject in need thereof,
b. administering an amount of a CaMKKβ targeting RNAi molecule to the subject in need thereof, or
c. a combination thereof.

In some embodiments, the AMPK alpha targeting RNAi molecule is capable of specifically binding a target sequence in an AMPK alpha gene product, the CaMKKβ targeting RNAi molecule is capable of specifically binding a CaMKKβ gene product, or both.

In some embodiments, the target sequence in the CaMKKβ gene product is
a. 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

In some embodiments, the target sequence in the AMPK alpha gene product is
a. 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

In some embodiments, the AMPK alpha gene product is an mRNA, wherein the CaMKKβ gene product is an mRNA, or both.

In some embodiments, the AMPK alpha gene product is an AMPK alpha isoform 1 encoding polynucleotide or an AMPK alpha isoform 2 encoding polynucleotide.

In some embodiments, the AMPK alpha targeting RNAi molecule is a polynucleotide having a sequence that
a. is 50-100 percent identical to a sequence selected from the group consisting of SEQ ID NOs: 25-72; or
b. is 50-100 percent identical to a sequence that is complementary to a sequence selected from the group consisting of SEQ ID NOs: 25-72.

In some embodiments, administering the AMPK alpha targeting RNAi molecule, the CaMKKβ targeting RNAi molecule, or both occurs
a. prior to exposure of the subject to a chemical insult, acoustic insult, or a combination thereof;
b. after exposure of the subject to a chemical insult, acoustic insult, or a combination thereof; or
c. both.

In some embodiments, administering the AMPK alpha targeting RNAi molecule, the CaMKKβ targeting RNAi molecule, or both occurs
a. prior to exposure of the subject to an ototoxic agent;
b. after exposure of the subject to an ototoxic agent; or
c. both.

In some embodiments, the ototoxic agent is a selected from the group consisting of: an antibiotic, a chemotherapeutic agent, a diuretic, a non-steroidal anti-inflammatory agent, an anti-malarial agent, an industrial solvent, an anticonvulsant agent, a psychopharmacologic agent, a cardiac or blood pressure therapeutic agent, an anti-allergy agent, a quinine-based agent, a glucocorticosteroid, an anesthetic, or a combination thereof.

In some embodiments, the ototoxic agent is an aminoglycoside antibiotic.

In some embodiments, the ototoxic agent is gentamicin, neomycin, kanamycin, amikacin, streptomycin, tobramycin, netilmicin, vancomycin, erythromycin, or a combination thereof.

In some embodiments, the ototoxic agent is a platinum-based chemotherapeutic agent.

In some embodiments, the ototoxic agent is cisplatin, carboplatin, oxaliplatin, nitrogen mustard, methotrexate, vincristine, dactinomycin, bleomycin, or any combination thereof.

In some embodiments, the diuretic is furosemide, bumetanide, ethacrynic acid, torsemide, chlor-thalidone, or any combination thereof.

In some embodiments, the non-steroidal anti-inflammatory agent is an acetic acid-based NSAID, a COX-2 inhibitor, a fenamate, an oxicam, a propionic acid, a salicylate, or a miscellaneous NSAID, or any combination thereof.

In some embodiments, the quinine-based agent is chloroquine phosphate, auinacrine hydrochloride, quinine sulfate, or any combination thereof.

In some embodiments, the anti-malarial agent is chloroquine and hydroxychloroquine.

In some embodiments, the cardiac or blood pressure therapeutic agent is celiprolol, flecaninide, lidocaine, metoprolol, procainamide, propranolol, or quinidine.

In some embodiments, the industrial solvent is cyclohexane, dichloromethane, hexane, indane, methyl-chloride, methyl-n-butyl-ketone, precholor-ethylene, styrene, tetrachlor-ethane, toluol, tricholorethylene, or any combination thereof.

In some embodiments, the anesthetic is bupivacaine, tetracaine, lidocaine, or a combination thereof.

In some embodiments, the glucocorticosteroid is prednisone, prednisolone, adrenocorticotrophic hormone, or any combination thereof.

In some embodiments, the psychopharmacologic agent is amitriptyline, a benzodiazepine, bupropion, carbamazepine, diclofensine, doxepin, desipramine, fluoxetine, imipramine, lithium, melitracen, molindone, paroxetine, phenelzine, protriptyline, trazodone, zimeldine, or any combination thereof.

In some embodiments, the chemical ototoxic agent is alcohol, arsenic or arsenic-based compound, caffeine, lead, marijuana, nicotine, mercury, or auranofin, or any combination thereof.

In some embodiments, the AMPK alpha targeting RNAi molecule, the CaMKKβ targeting RNAi molecule, or both is/are administered directly to an ear of a subject.

In some embodiments, the AMPK alpha targeting RNAi molecule, the CaMKKβ targeting RNAi molecule, or both is/are administered via intra-tympanic delivery, intracochlear delivery, semicircular canal delivery, or a combination thereof.

In some embodiments, the AMPK alpha targeting RNAi molecule, the CaMKKβ targeting RNAi molecule, or both is/are administered via intra-tympanic delivery on the round window membrane.

In some embodiments, the AMPK alpha targeting RNAi molecule, the CaMKKβ targeting RNAi molecule, or both is/are administered via posterior semicircular canal delivery.

Described in some embodiments herein are methods of reducing an amount of an AMPK alpha gene product, a CaMKKβ gene product, or both in a subject in need thereof that includes:
a. administering an amount of an AMPK alpha targeting RNAi molecule to the subject in need thereof,
b. administering an amount of a CaMKKβ targeting RNAi molecule to the subject in need thereof, or
c. a combination thereof.

In some embodiments, the AMPK alpha targeting RNAi molecule is capable of specifically binding a target sequence in an AMPK alpha gene product, the CaMKKβ targeting RNAi molecule is capable of specifically binding a CaMKKβ gene product, or both.

In some embodiments, the target sequence in the CaMKKβ gene product is
a. 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

In some embodiments, the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

In some embodiments, the AMPK alpha gene product is an mRNA, wherein the CaMKKβ gene product is an mRNA, or both.

In some embodiments, the AMPK alpha gene product is an AMPK alpha isoform 1 encoding polynucleotide or an AMPK alpha isoform 2 encoding polynucleotide.

In some embodiments, the AMPK alpha targeting RNAi molecule is a polynucleotide having a sequence that
a. is 50-100 percent identical to a sequence selected from the group consisting of SEQ ID NOs: 25-72; or is
b. 50-100 percent identical to a sequence that is complementary to a sequence selected from the group consisting of SEQ ID NOs: 25-72.

In some embodiments, the AMPK alpha targeting RNAi molecule, the CaMKKβ targeting RNAi molecule, or both is/are administered directly to an ear of a subject.

In some embodiments, the AMPK alpha targeting RNAi molecule, the CaMKKβ targeting RNAi molecule, or both is/are administered via intra-tympanic delivery, intracochlear delivery, semicircular canal delivery, or a combination thereof.

In some embodiments, the AMPK alpha targeting RNAi molecule, the CaMKKβ targeting RNAi molecule, or both is/are administered via intra-tympanic delivery on the round window membrane.

In some embodiments, the AMPK alpha targeting RNAi molecule, the CaMKKβ targeting RNAi molecule, or both is/are administered via posterior semicircular canal delivery.

### Co-Therapies

The pharmaceutical formulations provided herein can be administered in combinations with or include one or more other auxiliary agents. Suitable auxiliary agents are described elsewhere herein. In some embodiments, the auxiliary agent is an ototoxic agent. The compound(s), and/or formulation(s), and/or additional therapeutic agent(s) can be administered simultaneously or sequentially by any convenient route in separate or combined pharmaceutical formulations.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Noise Exposure and KM-Furosemide Treatment can Activate CaMKKβ

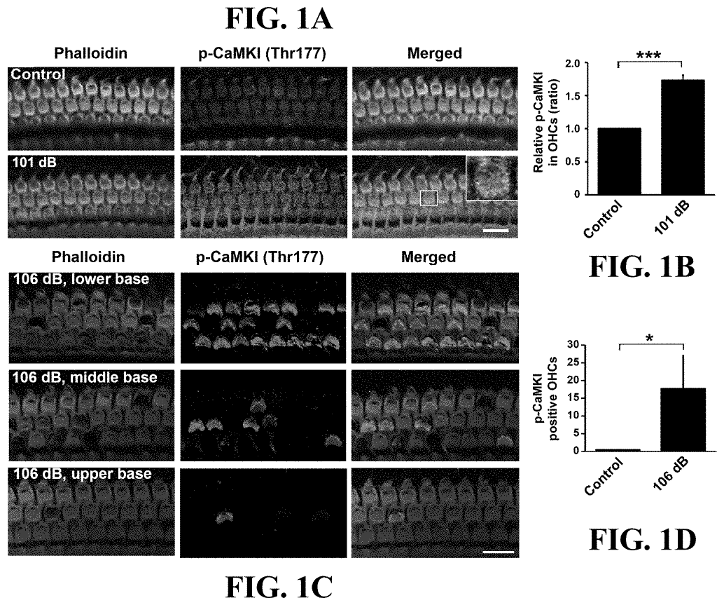

FIGS. 1A-1D can demonstrate that traumatic noise activates CaMKI in OHCs of the basal turn. (FIG. 1A) The p-CaMKI (Thr177, red) immunolabeling in OHCs of the basal turn was stronger examined 1 h after completion of 101-dB broad band noise (BBN) exposure than in unexposed controls. Phalloidin (green) staining for F-actin shows OHC structure. For better visualization of punctate labeling for p-CaMKI, one OHC was enlarged from the merged panel as indicated by the inset white box. These images were taken from the basal turn corresponding to the 22-32 kHz region and are representative of six mice in each group; scale bar=10 μm. (FIG. 1B) Semi-quantification of the p-CaMKI (Thr177) immunolabeling in OHCs in the 22-32 kHz region confirmed a significant increase. Data are presented as means+SD, n=6, ***p<0.001. (FIG. 1C) The basal region of the sensory epithelium displayed a tonotopic gradient with increasing numbers of OHCs positive for p-CaMKI (Thr177, red) immunolabeling farther from the apex 1 h after completion of exposure to 106-dB noise. The representative images were taken from the basal turn (lower base: 45-kHz, middle base: 32-kHz, and upper base: 22-kHz regions) and are representative of four mice for each group; scale bar=10 μm. (FIG. 1D) Counting the number of OHCs displaying p-CaMKI (Thr177) immunolabeling along the basal turn of the cochlear duct (3-5 mm from the apex) confirms a significant increase 1 h after completion of 106-dB noise exposure. Data are presented as means+SD, 106 dB: n=4; Control: n=6, *p<0.05.

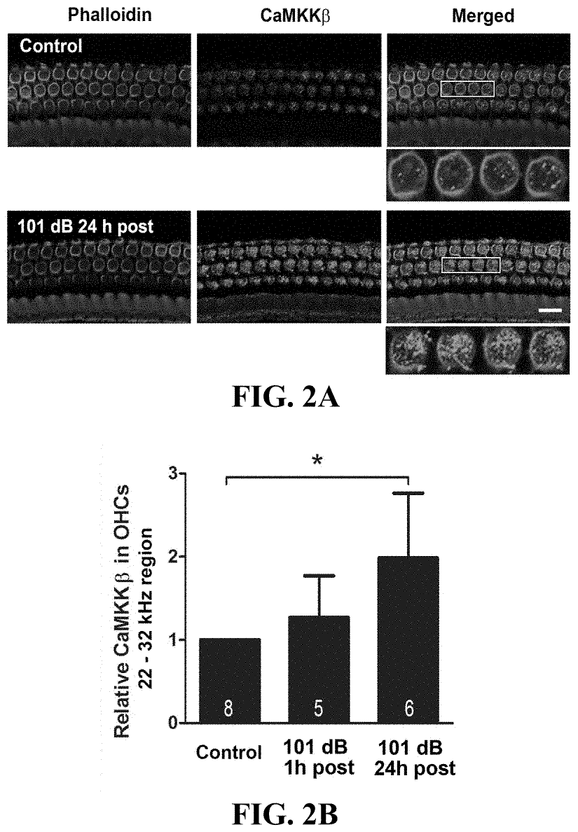

FIGS. 2A-2B can demonstrate that noise increases CaMKKβ expression in the cochlear outer hair cells. (FIG. 2A) Immunolabeling for CaMKKβ (red) in OHCs of the basal turn corresponding to sensitivity to 22-32 kHz was strong when processed 24 h after completion of the noise exposure compared to unexposed controls. Enlarged OHCs are shown for better visualization of punctate labeling for CaMKKβ; green: phalloidin-stained OHCs, scale bar=10 μm. (FIG. 2B) Semi-quantitative analysis of grayscale CaMKKβ labeling in OHCs confirmed a significant increase when examined at 24 h, but not at 1 h after the completion of the exposure. Data are presented as means+SD. The number of animals in each group is indicated in the bar graph, *p<0.05.

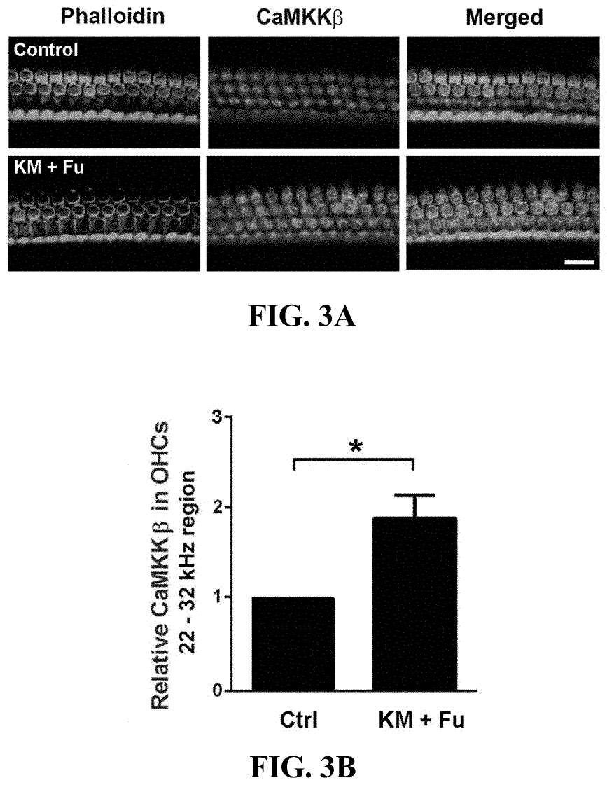

FIGS. 3A-3B can demonstrate that treatment with KM plus furosemide increases CaMKKβ expression in the cochlear outer hair cells. (FIG. 3A) Immunolabeling for CaMKKβ (red) in OHCs of the basal turn corresponding to sensitivity to 22-32 kHz was strong when processed 24 h after KM plus furosemide treatment compared to untreated mice. KM alone or furosemide treatment alone showed similar CaMKKβ immunolabeling as those of control mice. Green: phalloidin-stained OHCs, scale bar=10 μm. (FIG. 3B) Semi-quantitative analysis of grayscale CaMKKβ labeling in OHCs confirmed a significant increase. Data are presented as means+SD; n=5 mice, *p<0.05.

These results can evidence that noise exposure and KM-furosemide treatment induce activation of CaMKKβ in OHCs. As noise exposure and KM-furosemide treatment result in similar cochlear pathology, i.e. loss of sensory hair cells with OHCs more vulnerable than IHCs, these data support that they may have similar mechanisms.

### Example 2—Efficiency of siRNA can be Increased Via Posterior Semi-Circular Canal Delivery

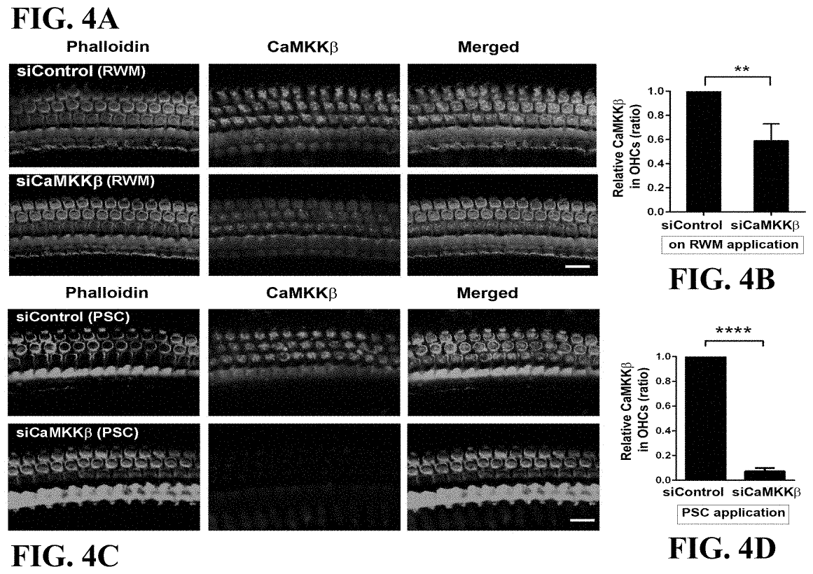

FIGS. 4A-4D can demonstrate that silencing efficiency between siCaMKKβ via intra-tympanic delivery onto the RWM and PSC delivery in OHCs. (FIG. 4A) Representative images show reduction of CaMKKβ-associated immunoreactivity in OHCs of the sensory epithelium 72 h after intra-tympanic delivery of siCaMKKβ onto the RWM of the left ears in mice when compared to those of scrambled RNA (siControl) mice. Images are representative of five individual mice for each condition. (FIG. 4B) Semi-quantification of CaMKKβ immunolabeling in grayscale in OHCs in the basal turn, corresponding to sensitivity to 22-32 kHz, confirms a significant decrease of about 40% in siCaMKKβ-treated cochleae compared to siControls. (FIG. 4C) Representative images show reduction of CaMKKβ-associated immunoreactivity in OHCs of the sensory epithelium 72 h after PSC delivery of siCaMKKβ to the left ears of mice when compared to those of siControl mice. Images are representative of five individual mice for each condition. Red: CaMKKβ; green: phalloidin-stained OHCs; scale bar=10 μm. (FIG. 4D) Semi-quantification of CaMKKβ immunolabeling in grayscale in OHCs in the basal turn, corresponding to sensitivity to 22-32 kHz, confirms a significant decrease of about 90% in siCaMKKβ-treated cochleae compared to siControls; siCaMKKβ: CaMKKβ siRNA, siControl: scrambled siRNA. Data are presented as means+SD, n=5 for each condition; **p<0.01, ****p<0.0001.

In agreement with others, delivery of naked siRNA can silence the protein expression in OHCs (Oishi et al., 2013; Yuan et al., 2015; Hill et al., 2016). Expanding upon these efforts, the results herein can demonstrate that the silencing efficiency of siCaMKKβ can be increased through PSC delivery. These results herein can demonstrate that silencing efficiency is significantly higher when delivered via PSC than via intra-tympanic delivery onto the RWM. Although RWM delivery can be efficacious, an advantage of PSC delivery of siRNA is that the amount reaching the cells of the inner can more easily determined and controlled because siRNA is applied directly to the inner ear endolymph.

### Example 3—Pretreatment with siCaMKKβ Via Intra-Tympanic Delivery can Attenuate Noise-Induced Loss of Synapses and Outer Hair Cells and Hearing Loss

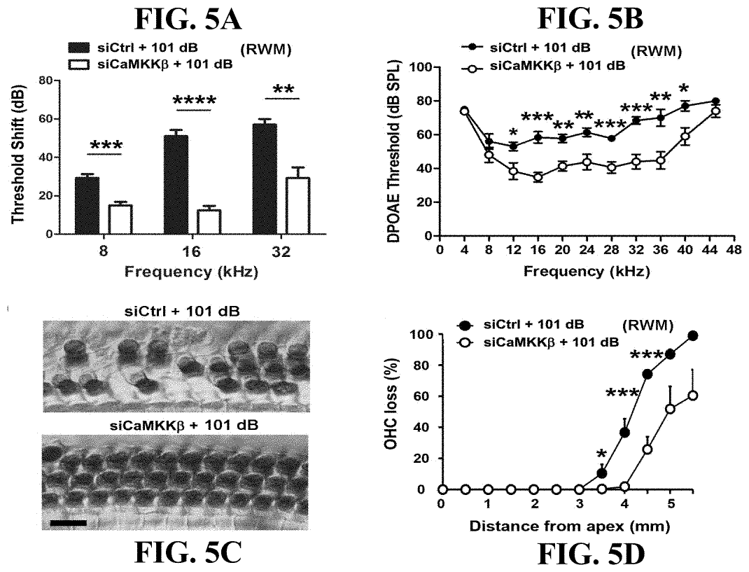

FIGS. 5A-5D can demonstrate that pretreatment with CaMKKβ siRNA via intra-tympanic delivery on RWM reduces NIHL assessed 14 d after noise exposure. (FIG. 5A) Pretreatment with siCaMKKβ significantly decreases threshold shifts induced by exposure to 101-dB noise at 8, 16, and 32 kHz. Data are presented as means+SD, n=10 mice in each group. (FIG. 5B) Pretreatment with siCaMKKβ significantly attenuates 101-dB-noise-increased DPOAE thresholds from 12-40 kHz, n=5 mice in each group. (FIG. 5C) Representative images revealed Myosin-VIIA-immunolabeled OHCs (brown) 14 d after 101-dB-noise exposure in groups pretreated with siControl or siCaMKKβ. Images were taken at 4 mm from the apex. Scale bar=10 μm. (FIG. 5D) Quantification of OHC numbers along the cochlear duct showed that pretreatment with siCaMKKβ significantly reduced noise-induced OHC loss. siControl+101 dB: n=5; siCaMKKβ+101 dB noise: n=7. Data are presented as means±SD, *p<0.05, **p<0.01, ****p<0.001, ****p<0.0001.

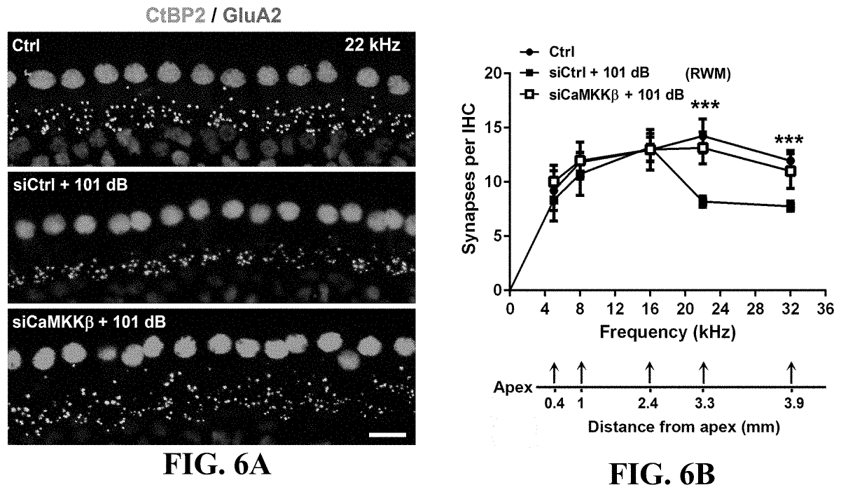

FIGS. 6A-6B can demonstrate that Pretreatment with siCaMKKβ via intra-tympanic delivery onto the RWM attenuates noise-induced loss of synapses. (FIG. 6A) Representative images of immunolabeling for CtBP2 (red)/GluA2 (green) in the 22-kHz region of cochlear epithelia 14 d after completion of 101-dB-noise exposure. Images were compressed from 30 Z-stack projections. Scale bar=10 μm. (FIG. 6B) Quantification of CtBP2/GluA2-immunolabeled synapse particles in IHCs corresponding to 6, 8, 16, 22, and 32 kHz showed that broad band noise exposure reduced loss of synapses at 22 and 32 kHz, while pretreatment with siCaMKKβ prevented noise-induced loss of synapses. The frequency correlation along the cochlear epithelium is indicated at the bottom. Data are presented as means±SD, n=5 in each noise-exposed group with siControl and siCaMKKβ, n=10 control mice without noise exposure, ****p<0.001.

### Example 4—Pretreatment with siCaMKKβ Via PSC Delivery can Prevent KM-Furosemide-Induced Loss of Outer Hair Cells and Hearing Loss

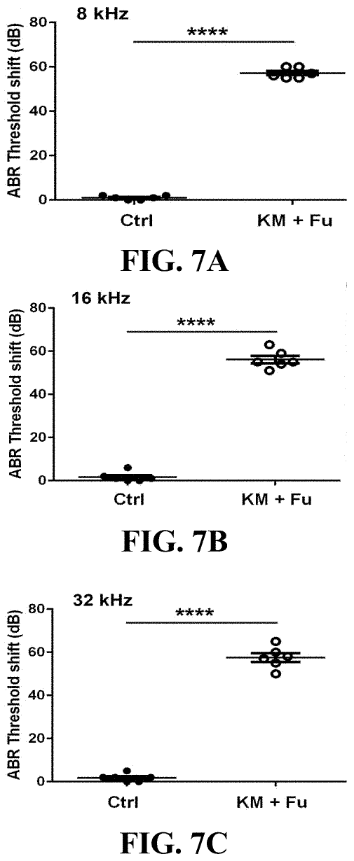

FIGS. 7A-7C can demonstrate a mouse model of kanamycin-plus-furosemide-induced hearing loss in CBA/J mice at the age of 6-7 weeks. A single treatment of kanamycin (KM) at 800 mg/kg subcutaneously (SQ) plus furosemide (Fu) at 200 mg/kg via intraperitoneal (IP) injection resulted in permanent auditory threshold shifts at 8, 16, and 32 kHz when measured 14 d after the injections. Data are presented as mean±SD, ****p<0.0001. One-time injection of KM or Fu alone does not induce hearing loss at any measured frequency.

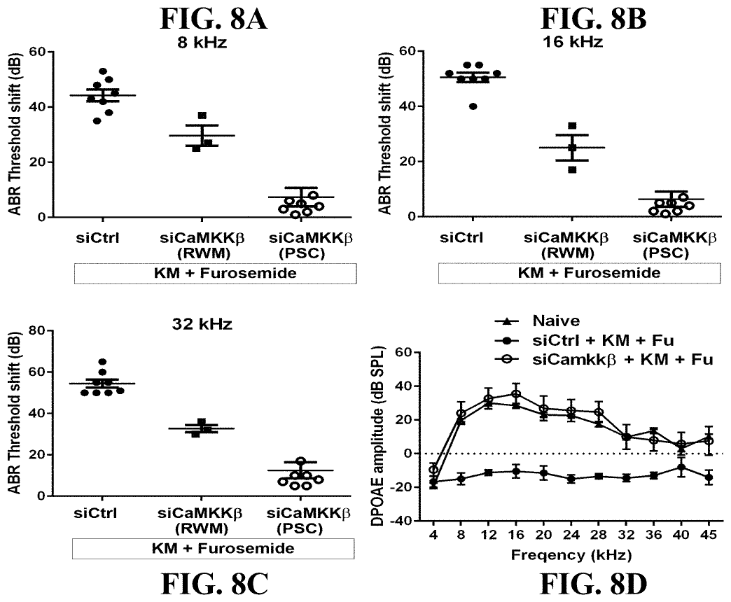

FIGS. 8A-8D can demonstrate that Pretreatment with CaMKKβ siRNA via intra-tympanic delivery onto the RWM attenuates auditory threshold shifted by co-treatment of KM with furosemide; pretreatment via PSC application completely prevents KM-furosemide-induced auditory threshold shifts and damage to OHC function when measured 14 d after the injection. (FIGS. 8A-8C) Pre-application of siCaMKKβ through delivery onto the RWM resulted in significant reduction of KM-furosemide-induced auditory threshold shifts at 8, 16, and 32 kHz. Surprisingly, delivery of siCaMKKβ through the PSC completely prevented KM-furosemide-induced-auditory threshold shifts at all measured frequencies (8, 16, and 32 kHz). (FIG. 8D) OHC functional measurement by DPOAE showed that pretreatment with siCaMKKβ via the PSC also completely prevented reduction of DPOAE by co-treatment of KM-furosemide. Data are presented as means+SEM, Naive: n=13, siControl+KM-furosemide: n=8, siCaMKKβ+KM-furosemide: n=7. All ARBs and DPOAEs were measured on the left ears of mice. Statistical analysis between siCtrl+KM+Fu and siCaMKKβ+KM+Fu were analyzed by repeated measures ANOVA. F 1, 10=87.831, p<0.0001.

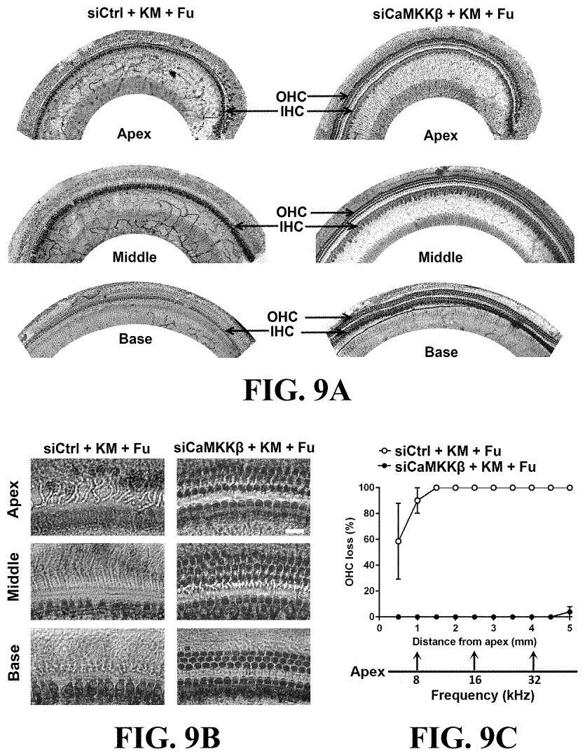

FIGS. 9A-9C can demonstrate that pretreatment with CaMKKβ siRNA via PSC application completely prevents KM-furosemide-induced OHC loss when measured 14 d after the lesions. (FIGS. 9A-9B) Representative images of the apex, middle, and basal turns display myosin-VIIa-labeled sensory hair cells in siCtrl+KM+Fu and siCaMKKβ+KM+Fu groups by 10× magnification (FIG. 9A) or 63× magnifications, scale bar=10 μm (FIG. 9B). (FIG. 9C) Hair cells were counted along the entire length of the cochlear spiral. The distances along the cochlear duct that correlate with the frequencies of 8, 16, and 32 kHz are indicated. Data are presented as means±SEM and analyzed by repeated measures ANOVA. F_{1, 13}=742.84, p<0.0001. One cochlea was used per mouse.

These results can demonstrate that targeting CaMMKβ can prevent outer hair cell loss and/or hearing loss stemming from insults such as aminoglycosides and noise.

### Example 5—Materials, Methods, and References for Examples 1-4

### Animals

Male CBA/J mice at 10 weeks of age for noise experiments and at 4 weeks of age for kanamycin-furosemide experiments were purchased from The Jackson Laboratory. All mice had free access to water and a regular mouse diet (Irradiated Lab Diet #5V75) and were kept at 22±1° C. under a standard 12:12 h light-dark cycle to acclimate for at least 1 week before conducting baseline auditory brainstem response (ABR) measurements. All mice were housed in the animal facility of the Children's Research Institute at the Medical University of South Carolina. All research protocols were approved by the Institutional Animal Care and Use Committee at MUSC. Animal care was under the supervision of the Division of Laboratory Animal Resources at MUSC.

### Noise Exposure

Unrestrained CBA/J male mice at the age of 12 weeks (one mouse per stainless steel wire cage, approximately 9 cm³) were exposed to broad band noise from to 2-20 kHz at 101 dB sound pressure level (SPL) for 2 h to induce permanent threshold shifts with loss of synaptic ribbons and OHCs, but not IHCs, or 106 dB to induce permanent threshold shifts with losses of ribbons, OHCs, and IHCs by 14 d after the noise exposure. The sound exposure chamber was fitted with a loudspeaker (model 2450H; JBL) driven by a power amplifier (model XLS 202D; Crown Audio) fed from a CD player (model CD-200; Tascam TEAC American). Audio CD sound files were created and equalized with audio editing software (Audition 3; Adobe Systems, Inc.). The background sound intensity of the environment surrounding the cages was 65 dB as measured with a sound level meter (model 1200; Quest Technologies). Sound levels for noise exposure are calibrated with a sound level meter at multiple locations within the sound chamber to ensure the uniformity of the sound field before and after exposure. Control mice were kept in silence (without use of the loudspeaker) within the same chamber for 2 h.

### Kanamycin-Furosemide Treatment

Kanamycin sulfate (USB, #25389-94) dissolved in 0.9% saline at dose of 800 mg base/kg was injected SQ to mice 1 h following a single intraperitoneal injection (IP) of furosemide at 200 mg/kg (Baxter, injection, #AIN00218). Each animal received injections at a volume of 200-300 μL each.

Intra-Tympanic Delivery of siRNA onto the RWM or Posterior Semicircular Canal (PSC) Delivery of siRNA

siCaMKKβ (Thermo Fisher, #n437808) (sense: GAGCACAAAUAAUUGUUUUTT SEQ ID NO: 1; antisense AAAACAAUUAUUUGUGCUCTA SEQ ID NO: 2) or siControl (Thermo Fisher, #4390844) was locally delivered via intra-tympanic application onto the RWM as previously described (Oishi et al., 2013; Hill et al., 2016) or via PSC to the endolymph based on reports (Guo, 2018 #3975; Tao, 2018 #3973). Briefly, after anesthesia, a retroauricular incision was made to approach the temporal bone. For intra-tympanic delivery onto the RWM, the otic bulla was identified ventral to the facial nerve and a shallow hole was made in the thin part of the otic bulla with a 30-G needle and enlarged with forceps to a diameter of 2 mm in order to visualize the round window. A customized sterile micro medical tube was inserted into the hole just above the round window niche (RWN) to slowly deliver 10 μL (0.6 μg) of siCaMKKβ or siControl. For PSC delivery, the stemocleidomastoid muscle was separated to expose the PSC. A small hole was made with the tip of a 30-G needle on the PSC and left open for about 1 min (there was no obvious endolymph leakage). Then the tip of the polyimide tube was inserted into the PSC toward the ampulla, and 1 μL of siRNA was slowly microinjected for about 10 minutes. After the siRNA delivery, the hole was covered with surrounding muscle and glued with tissue adhesive. Finally, the skin incision was closed with tissue adhesive and the mouse was kept in the surgical position (less than 1 h) until it had woken up. Seventy-two hours after siRNA delivery, the animals were exposed to noise for 2 hours. Forty-eight hours after siRNA delivery, the animals were treated once with KM and furosemide.

### Auditory Brainstem Response and DPOAE Measurements

ABRs were measured in the left ears of anaesthetized mice before and two weeks after noise exposure. Mice were anesthetized with an intraperitoneal injection of a mixture of ketamine (100 mg/kg) and xylazine (10 mg/kg), and then placed in a sound-isolated and electrically shielded booth (Acoustic Systems). Body temperature was monitored and maintained near 37° C. with a heating pad. Acoustic stimuli were delivered monaurally to a Beyer earphone attached to a customized plastic speculum inserted into the ear canal. Subdermal electrodes were inserted at the vertex of the skull (active), mastoid region under the left ear (reference), and mastoid region under the right ear (ground). ABRs were measured at 8, 16, and 32 kHz. Tucker Davis Technology (TDT) System III hardware and SigGen/Biosig software were used to present the stimuli (15 ms duration tone bursts with 1 ms rise-fall time) and record the response. Up to 1024 responses were averaged for each stimulus level. ABR wave I was used to determine ABR thresholds for each frequency. Thresholds were determined for each frequency by reducing the intensity in 10-dB increments and then in 5-dB steps near threshold until no organized responses were detected. Thresholds were estimated between the lowest stimulus level where a response was observed and the highest level without response. All ABR measurements were conducted by the same experimenter. The ABR values were assigned by an expert who was blinded to the treatment conditions. Distortion product optoacoustic emissions (DPOAE) testing was performed in mice under anesthesia described above for ABRs. The emissions were measured using TDT RZ6 System and SigGen software. The acoustic assembly containing an ER-10B+ microphone connected to two transducers was tightly apposed into the ear canal. Primary tones were presented at fixed intensity levels of L1=65 dB SPL and L2=55 dB SPL with f2/f1 ratio of 1.2. Physiological results of each mouse were analyzed for individual frequencies, and then averaged for each of these frequencies from 4 kHz to 45 kHz.

### Immunocytochemistry for Cochlear Surface Preparations

The temporal bones were removed and perfused locally with a solution of 4% paraformaldehyde in PBS, pH 7.4, and kept in this fixative overnight at 4° C. For immunolabeling of IHC synapses, the cochleae were perfused locally with a solution of 4% paraformaldehyde in PBS, pH 7.4, and kept in this fixative for 1.5 h at room temperature. Between every step, the cochlear samples were washed at least three time with PBS for 5-10 min each wash. After decalcification with 4% sodium EDTA solution (adjusted with HCl to pH 7.4) for 3 days at 4° C., the cochleae were micro-dissected into three turns (apex, middle, and base) and adherened to 10-millimeter round coverslips (Microscopy Products for Science and Industry, cat #260367) with cell-Tak (BD Biosciences, cat #354240). The specimens were first permeabilized in 3% Triton X-100 solution and then blocked with 10% normal goat serum each for 30 min at room temperature, followed by incubation with primary antibodies: polyclonal rabbit CaMKKβ at 1:200 (Invitrogen #PA5-30399, and #PA5-30558) or polyclonal rabbit anti-p-CaMKI (Thr 177) at 1:50 (Santa Cruz #sc-28438-R) at 4° C. for 48 h. The specimens were then incubated with the Alexa Flour 594-conjugated secondary antibody at a concentration of 1:200 at 4° C. overnight and followed with incubation with Alexa Flour 488-phalloidin for 1 h at room temperature in darkness. Control incubations were routinely processed without primary antibody treatments.

For immunolabeling IHC synapses, the specimens were incubated in darkness at 37° C. overnight with primary monoclonal mouse anti-CtBP2 IgG1 at 1:200 (BD Biosciences, #612044) and mouse anti-GluA2 IgG2a at 1:2,000 (Millipore, MAB397) followed with the Alexa Fluor 594 goat anti-mouse IgG1 and Alexa Fluor 488 goat anti-mouse IgG2a (1:1,000) at 37° C. for 1 h in darkness and then the step for secondary antibody labeling was repeated based on previous reports (Hill, 2016 #3963; Wan, 2014 #3819). For visualization of sensory hair cells, the specimens were incubated with primary antibody polyclonal rabbit anti-myosin VIIa (Proteus Biosciences, 25-6790) at 1:200 in darkness at 4° C. overnight, followed by incubation with Alexa Fluor 350 secondary antibody at a concentration of 1:200 at 4° C. overnight in darkness.

All immunolabeling samples on round coverslips, after at least three final washes with PBS, were mounted using Fluoro-gel with Tris buffer (Electron Microscopy Sciences, #17985-10) with another round coverslip to sandwiches the samples and placed on slides. Finally, samples were sealed with nail polish. Immunolabeled images were taken with a 63×-magnification lens under identical Z-stack conditions using a Zeiss LSM 880 microscope.

Unless otherwise specified, all chemicals and reagents used were purchased from Sigma Aldrich.

Semi-Quantification of the Immunofluorescence Signals from Surface Preparations

Immunohistochemistry is well accepted as a semi-quantitative methodology when used with careful consideration of the utility and semi-quantitative nature of these assays (Taylor, 2006 #3978; Walker, 2006 #3979). The specificity of antibodies must be first detected by Western blot analysis. Antibodies showing only a single band with the correct molecular weight were used for immunolabeling on surface preparations and quantification of signaling in OHCs. The regions of interest were outlined within individual OHCs based on the counterstaining. The grayscale value was determined in only the hair cells to quantify the change in fluorescence intensity. This procedure provided quantitative measurements that are not confounded by protein expression in other cell types of the cochlea.

Immunolabeling for p-CaMKI and CaMKKβ was quantified from original confocal images, each taken with a 63×-magnification lens under identical conditions and equal parameter settings for laser gains and PMT gains, using ImageJ software (National Institutes of Health, Bethesda, Md.). The cochleae from the different groups were fixed and stained simultaneously with identical solutions and processed in parallel. All of the surface preparations were counter-stained with Alexa Fluor 488 phalloidin (green) to label hair cell structure in order to identify the comparable parts of the hair cells in confocal images. The regions of interest of individual OHCs were outlined with the circle tool based on phalloidin staining. The immunolabeling for the target proteins were measured in the upper-basal region (corresponding to sensitivity to 22-32 kHz) of cochlear surface preparations in 0.12-mm segments, each containing about 60 OHCs. The intensity of the background was subtracted and average grayscale intensity per cell was then calculated. For each repetition, the relative grayscale value was determined by normalizing the ratio to control.

There were no obvious changes in the levels of p-CaMKI and CaMKKβ protein in OHCs of the apical and middle turns between the ears of control mice and those exposed to BBN. The changes observed occurred only in the basal turn when assessed 1 h or 24 h after the exposure.

Counting the Immunolabeled Ribbons from Z Projections on Surface Preparations

A procedure as previously described by Hill et al., 2016 was followed. Immunofluorescence of CtBP2 on surface preparations was quantified from original confocal images, each taken with a 63×-magnification lens under identical Z-stack conditions in 0.25-mm intervals and equal parameter settings for laser gains and PMT gains. The z-stack images in each 0.12-mm segment (containing about 16 IHCs) were captured from cochlear surface preparations. The number of synaptic ribbons was counted using ImageJ software (National Institutes of Health, Bethesda, Md.). Briefly, the background of the images was subtracted, the noise was despeckled once, and the threshold was set to isolate the immunolabeling of ribbon signals. The image was then converted to a binary file and the number of ribbon particles was counted using the 3D Object Counter and divided by the total number of IHC nuclei within the image.

### Surface Preparations and DAB Staining of Cochlear Epithelia for Hair Cell Counts

Following decalcification and cochlear micro-dissection, the specimens were permeabilized in 3% Triton X-100 solution, blocked with 10% normal goat serum for 30 min at room temperature, incubated with a primary antibody (rabbit polyclonal anti-myosin VII at a 1:100 dilution) overnight at 4° C., and then incubated overnight at 4° C. with biotinylated goat anti-rabbit antibody at a 1:100 dilution. The specimens were washed with PBS three times between each step and then incubated in ABC solution (Vector Laboratories #PK-4001) overnight. Following another washing, the cochleae were incubated in DAB for 3 h, as necessary for sufficient staining intensity, followed by washing to stop the DAB reaction. Finally, the specimens were mounted on slides with mounting media. Images were taken with a Zeiss AxioCam MRc5 camera with Axioplan 2 imaging software under a Zeiss microscope.

Images from the apex through the base of the DAB-stained preparations were captured using a 20× lens on the Zeiss microscope. The lengths of the cochlear epithelia were measured and recorded in millimeters. OHCs were counted from the apex to the base along the entire length of the mouse cochlear epithelium. The percentage of hair cell loss in each 0.5-mm length of epithelium was plotted as a function of the cochlear length as a cytocochleogram (Chen, 2012 #3962; Zheng, 2014 #3980).

Mapping of frequencies as a function of distance was calculated with the equation [d (%)=156.5-82.5×log (f)] from Müller's paper (Muller, 2005 #3981) and agreed with the literature (Viberg, 2004 #3982).

### Statistical Analyses

Data were analyzed using IBM SPSS Statistics Premium V21 and GraphPad software for Windows. Biological sample sizes were determined based on the variability of measurements and the magnitude of the differences between groups, as well as experience from our previous studies, with stringent assessments of difference. Data of OHC loss along the length of the cochlear spiral were analyzed with repeated measures one-way analysis of variance (ANOVA) with Tukey's multiple comparisons using IBM SPSS Statistics. The remaining analysis was done using GraphPad. Differences with multiple comparisons were evaluated by one-way ANOVA with multiple comparisons. Differences for single-pair comparisons were analyzed using two-tailed unpaired Student's t-tests. Data for relative ratios of single-pair comparisons were analyzed with one-sample t-tests. A p-value <0.05 was considered statistically significant. Data are presented as means±SD or SEM based on the sample size and variability within groups. Sample sizes are indicated for each figure.

### Example 6—Silencing siCaMKKβ does not Influence Outer Hair Cell Mechanotransduction Channels

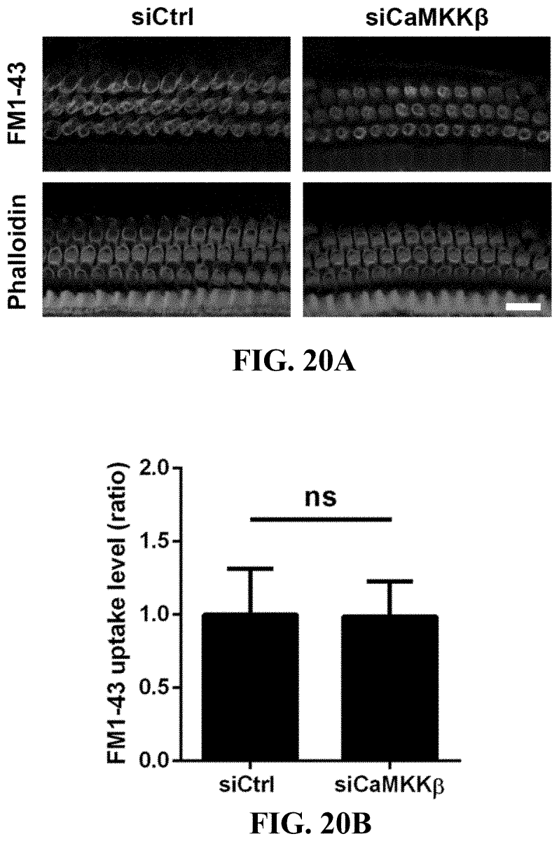

Since aminoglycoside antibiotics are known to enter outer hair cells (OHCs) mainly through mechanotransduction channels (MTCs), to address if protective effects of pretreatment with siCaMKKβ is due to blockade of kanamycin and/or furosemide entering OHCs, we assessed OHC MTCs using FM1-43FX, based on prior literature (Meyers et al., 2003; Taylor et al., 2008; Nakanishi et al., 2018). siCaMKKβ was delivered through the posterior semicircular canal (PSC) 48 h before application of FM1-43 to measure MTC function in OHCs. These results showed that pretreatment with siCaMKKβ via the PSC does not change FM1-43 uptake into OHCs compared with that of the siControl mice. FIG. 20A shows images of green fluorescence that reveal uptake of FM1-43 in OHCs. Scale bar=10 μm. FIG. 20B demonstrates a semi-quantitative analysis of the grayscales of green fluorescence intensity that shows no significant difference between siControl and siCaMKKβ groups. Data are presented as means+SD; ns: not significant. These results demonstrate that pretreatment with siCaMKKβ does not influence OHC mechanotransduction channel function and indicate that the protective effect against kanamycin-furosemide-induced loss of OHCs by siCaMKKβ is not due to inhibition of KM uptake.

### Example 7—Characterization of KM-Furosemide-Treatment Induced Hearing Loss in CBA/J Mice at 6-7 Weeks of Age

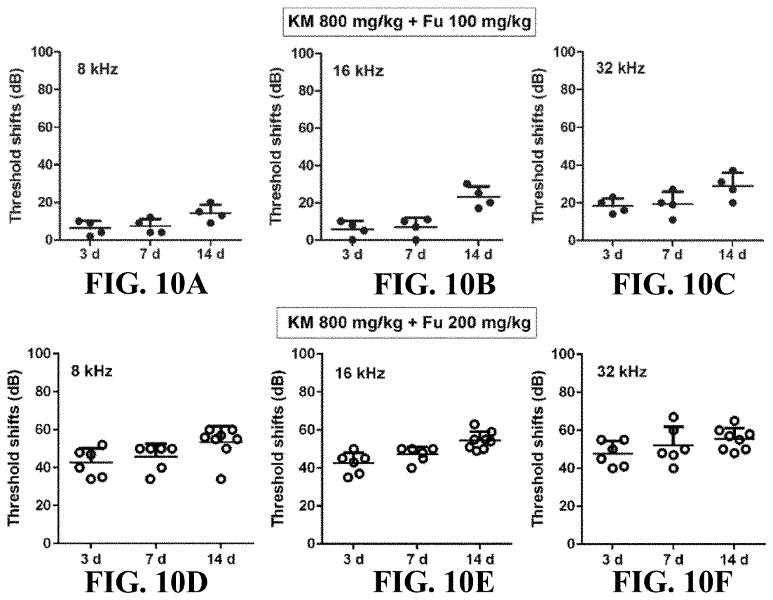

FIGS. 10A-10F can demonstrate a mouse model of kanamycin-plus-furosemide-induced hearing loss in CBA/J mice at the age of 6-7 weeks. FIGS. 10A-10C shows results of a single treatment of kanamycin (KM) at 800 mg/kg SQ plus furosemide (Fu) at 100 mg/kg via IP injection resulted in auditory threshold shifts of 20-30 dB at 8, 16, and 32 kHz when measured 14 d after the injections. FIGS. 10D-10F shows results from a single treatment of KM at 800 mg/kg SQ plus furosemide at 200 mg/kg via IP injection resulted in auditory threshold shifts of 50-60 dB at 8, 16, and 32 kHz when measured 14 d after the treatment. Data are presented as individual points with means+SD. These results can demonstrate that the dose of KM at 800 mg/kg plus furosemide at 200 mg/kg is suitable for our investigation.

### Example 8—KM-Furosemide Treatment Induces Activation of AMPKα in Outer Hair Cells

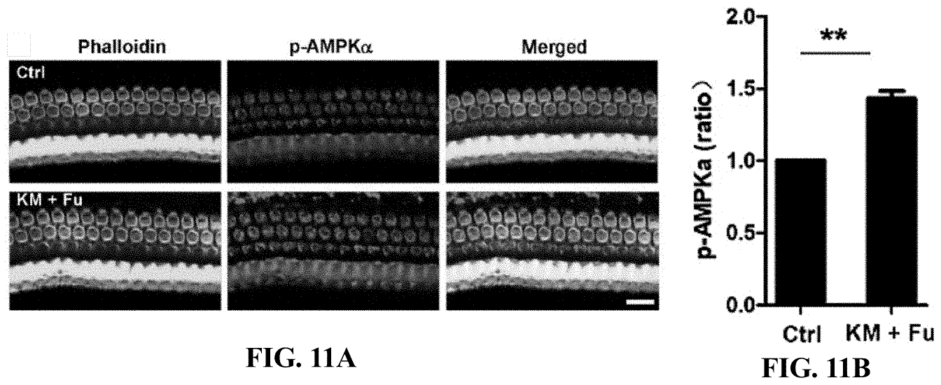

FIGS. 11A-11B can demonstrate that treatment with KM plus furosemide increases activation of AMPKα in cochlear outer hair cells. FIG. 11A shows results from immunolabeling for p-AMPKα in OHCs of the basal turn corresponding to sensitivity to 22-32 kHz was strong when processed 24 h after KM plus furosemide treatment compared to untreated mice in greyscale. Greyscale: phalloidin-stained OHCs, scale bar=10 μm. FIG. 11B shows results of a semi-quantitative analysis of immunolabeling for p-AMPKα (converted in grayscale) in OHCs confirmed a significant increase. Data are presented as means+SD; n=5 mice, **p<0.01. These results indicate that KM-furosemide treatment induces activation of AMPKα in OHCs.

### Example 9—Delivery of siAMPKα can Attenuate and/or Prevent Chemically Induced Cell Damage and/or Auditory Function

This Example can at least demonstrate (1) Intra-tympanic delivery of siAMPKα onto the RWM of the middle ear can attenuate KM-plus-furosemide-induced auditory threshold shifts with large variation; and (2) delivery of siAMPKα via PSC application completely prevents KM-furosemide-induced auditory threshold shifts, damage to OHC function, and loss of OHCs.

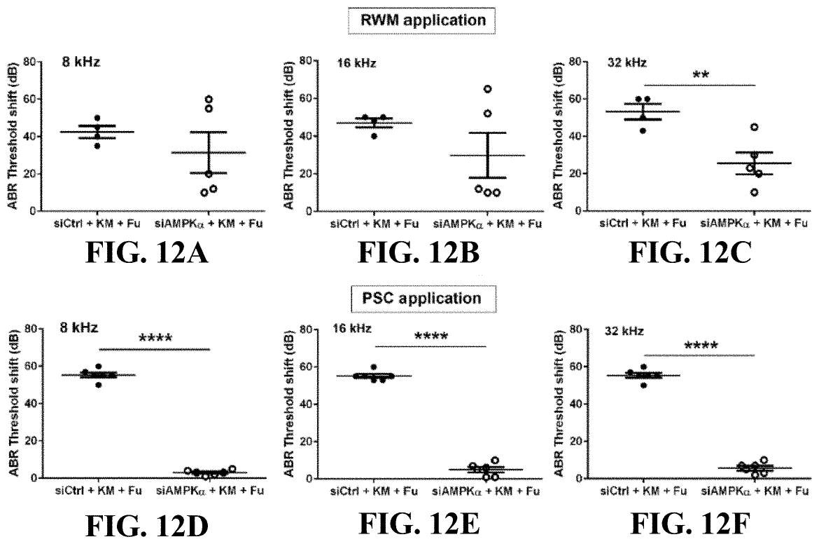

FIGS. 12A-12F can demonstrate that pretreatment with AMPKα siRNA via intra-tympanic delivery onto the RWM attenuates auditory threshold shifts from co-treatment of KM with furosemide with large variation, significant at 32 kHz (FIGS. 12A-12C). Pretreatment with AMPKα siRNA via PSC application completely prevents KM-furosemide-induced auditory threshold shifts at all three measured frequencies when measured 14 d after the injection (FIGS. 12D-12F). All ARBs were measured on the left ears of mice. Data are presented as individual points with means±SD, **p<0.01, ****p<0.0001.

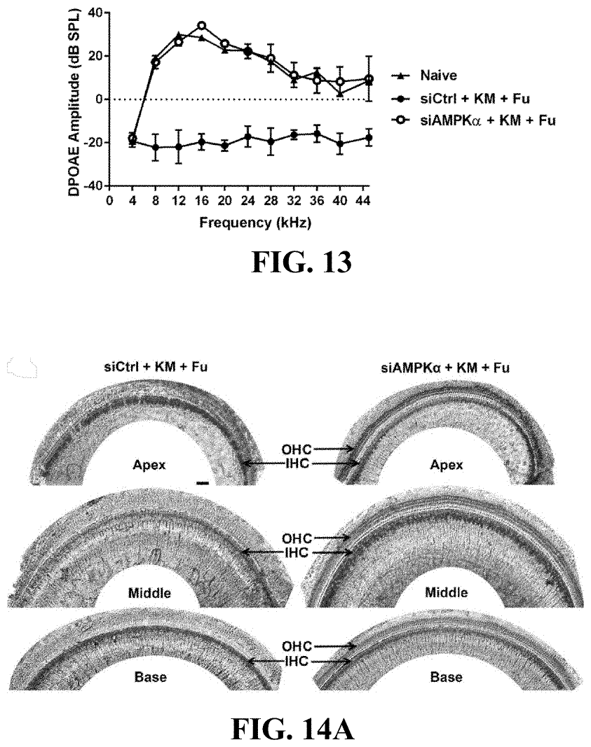

FIG. 13 can demonstrate that delivery of AMPKα siRNA via PSC application completely prevents KM-furosemide-induced damage to OHC function measured by DPOAE 14 d after the injection. Data are presented as means±SD; Naive: n=13, siControl+KM-furosemide: n=6, siAMPKα+KM-furosemide: n=6. DPOAEs were measured on the left ears of the mice. Statistical analysis between siCtrl+KM+Fu and siAMPKα+KM+Fu were performed by repeated measures ANOVA. F 1, 9=389.18, p<0.0001.

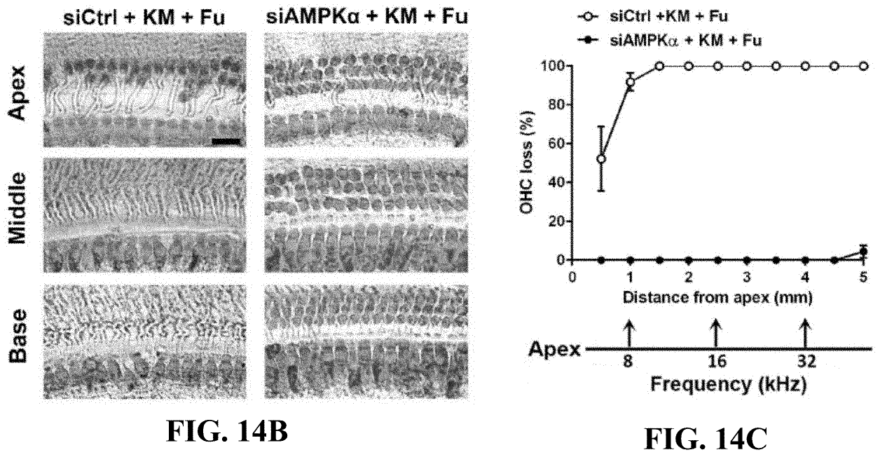

FIGS. 14A-14C can demonstrate that pretreatment with AMPKα siRNA via PSC application completely prevents KM-furosemide-induced OHC loss when measured 14 d after the lesions. FIG. 14A-14B show representative images of the apex, middle, and basal turns display myosin-VIIa-labeled sensory hair cells in siCtrl+KM+Fu and siAMPKα+KM+Fu groups by 10× (FIG. 14A) and 63× magnifications (FIG. 14B). FIG. 14C show results from counting hair cells along the entire length of the cochlear spiral. The distances along the cochlear duct that correlate with the frequencies of 8, 16, and 32 kHz are indicated. Data are presented as means±SD and analyzed by repeated measures ANOVA. F1, 14=1279.06, p<0.0001. One cochlea was used per mouse.

### Example 10—Characterization of Cisplatin/Furosemide-Treatment-Induced Hearing Loss and Hair Cell Loss in CBA/J Mice at 6-7 Weeks of Age

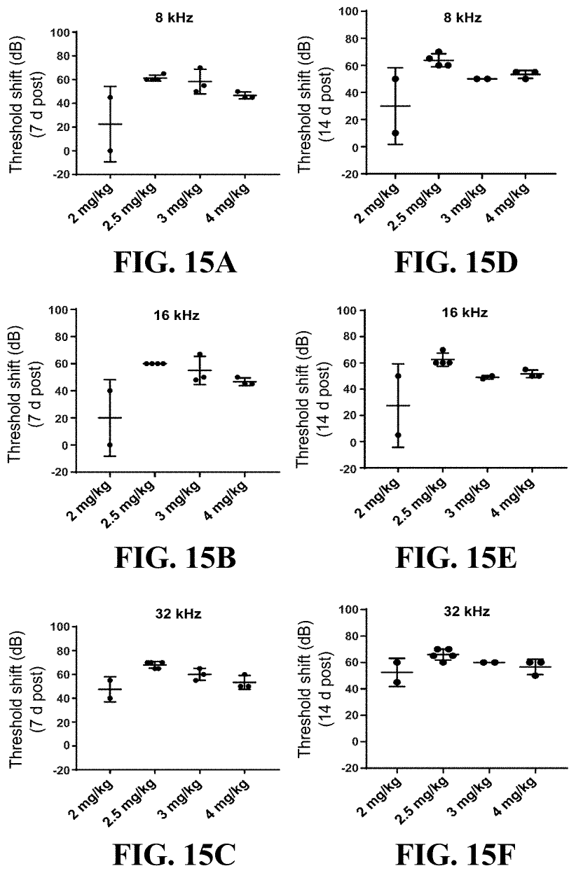

FIGS. 15A-15F demonstrates a mouse model of cisplatin-plus-furosemide-induced hearing loss in CBA/J mice at the age of 6-7 weeks. FIGS. 15A-15C can demonstrate that treatment with cisplatin (CDDP) at doses from 2-4 mg/kg plus furosemide at 200 mg/kg via IP injection for 2 days resulted in auditory threshold shifts measured at 8, 16, 32 kHz when measured on day 7 after treatment. FIGS. 15D-15F can demonstrate that auditory threshold shifts were maintained at 8, 16, 32 kHz when measured on day 14 after the treatment. Data are presented as individual points with means±SD.

These results can at least demonstrate that either concentration of CDDP can cause serious hearing loss, but the variation is larger when the mice are treated with 2 mg/kg CDDP. There is no difference in auditory threshold shifts between CDDP 2.5-4 mg/kg with 55-60 dB shifts when measured on day 7 and on day 14 post treatment. One mouse of the 3 mg/kg CDDP treated group died on day 8 after the treatment. This model was used to test the effect of AMPKα siRNA.

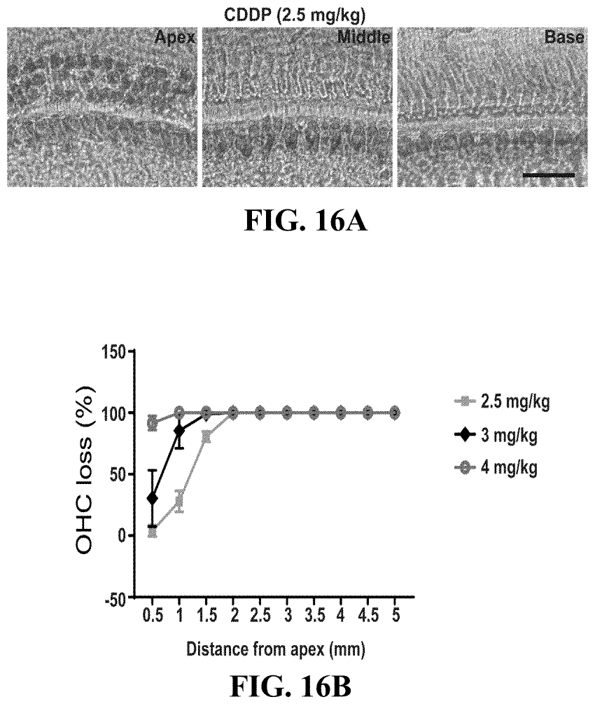

FIGS. 16A-16B can demonstrate that treatment with CDDP/furosemide causes massive loss of outer hair cells (OHCs) in mice when examined 14 d after the treatment. (FIG. 16A) Representative images of myosin-VIIa-labeled and DAB-stained apical, middle, and basal turns of surface preparations treated with 2.5 mg/kg CDDP. Scale bar=20 μm. (FIG. 16B) Quantitative analysis of OHC loss revealed complete loss of OHCs along the entire length of cochlear spiral with CDDP at 4 mg/kg plus furosemide at 200 mg/kg treatment and complete loss of OHCs in middle and basal turns with CDDP doses from 2.5-3 mg/kg. Data are presented as means±SD; CDDP 2.5 mg/kg: n=5, CDDP 3 mg/kg: n=2, CDDP 4 mg/kg: n=3.

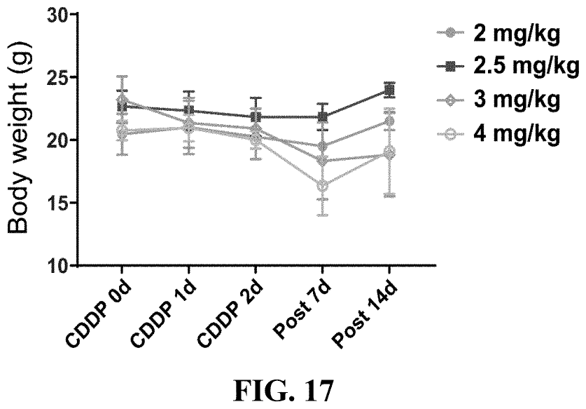

FIG. 17 can demonstrate changes in body weight with difference doses of CDDP treatment. Body weight was measured before treatment, on treatment days, and 7 and 14 d post treatment. There are no significant changes in body weight with treatment of CDDP at doses below or equal 2.5 mg/kg, but loss of body weight appeared on day 7 post CDDP treatment at and above 3 mg/kg doses compared with beginning of the treatment. Data are presented as means±SD. CDDP2 mg/kg: n=2, CDDP 2.5 mg/kg: n=5, CDDP 3 mg/kg: n=2, CDDP 4 mg/kg: n=3.

### Example 11—Delivery of siAMPKα Via PSC Application can Significantly Attenuate CDDP/Furosemide-Induced Auditory Threshold Shifts and Loss of OHCs

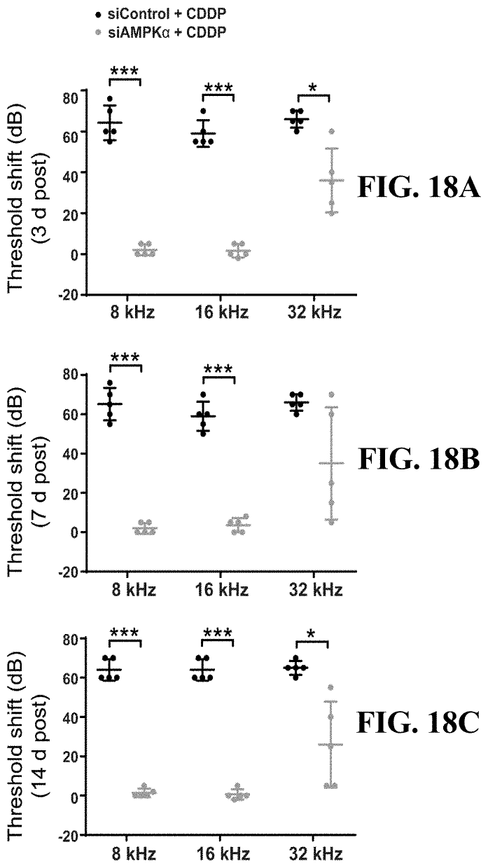

FIGS. 18A-18C can demonstrate that pretreatment with AMPKα siRNA (0.6 μg) via PSC application completely prevented CDDP-induced auditory threshold shifts at 8 and 16 kHz when measured on day 3 post (A) day 7 post (B) as well as day 14 post (C) compared with scrambled (siControl) mice. CDDP-induced auditory threshold shifts were partially attenuated at 32 kHz with huge variations between each mouse. CDDP was used at 2.5 mg/kg plus 200 mg/kg furosemide. Data are presented as individual points with means±SD, **p<0.01, ****p<0.0001.

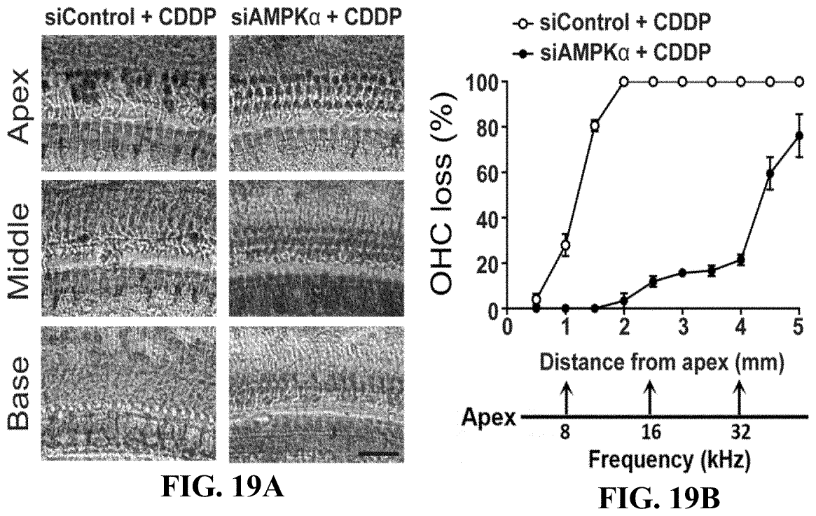

FIGS. 19A-19B can demonstrate that pretreatment with AMPKα siRNA via PSC application significantly attenuated CDDP/furosemide-induced outer hair cell loss when measured 14 d after the treatment. (A) Representative images from basal turn of surface preparations corresponding to sensitivity at 32 kHz region show myosin-VIIa-labeled and DAB-stained sensory hair cells in siControl+CDDP and siAMPKα+CDDP. CDDP: 2.5 mg/kg; furosemide: 200 mg/kg; Scale bar=20 μm. (B) Outer hair cells were counted along the entire length of the cochlear spiral revealing significant protection from hair cell damage with AMPKα siRNA treatment. Data are presented as means±SD, n=5 in each group.

### Example 12—Methods for Examples 7-11

### Animals

Male CBA/J mice at 4 weeks of age for kanamycin-furosemide experiments were purchased from The Jackson Laboratory. All mice had free access to water and a regular mouse diet (Irradiated Lab Diet #5V75) and were kept at 22±1° C. under a standard 12:12 h light-dark cycle to acclimate for at least 1 week before conducting baseline auditory brainstem response (ABR) measurements. All mice were housed in the animal facility of the Children's Research Institute at the Medical University of South Carolina. All research protocols were approved by the Institutional Animal Care and Use Committee at MUSC. Animal care was under the supervision of the Division of Laboratory Animal Resources at MUSC.

### Kanamycin-Furosemide Treatment

Kanamycin sulfate (USB Corporation, #25389-94) dissolved in 0.9% saline at dose of 800 mg base/kg was subcutaneously injected (SQ) to 5-6 week old mice 1 h following a single intraperitoneal injection (IP) of furosemide at 200 mg/kg (Baxter, injection, #AIN00218). Each animal received injections at a volume of 200-300 μL each.

Intra-Tympanic Delivery of siRNA onto the RWM or Posterior Semicircular Canal (PSC) Delivery of siRNA

siAMPKα (Thermo Fisher, #4390771) or siControl (Thermo Fisher, #4390844) was locally delivered via intra-tympanic application onto the RWM as previously described (Oishi et al., 2013; Hill et al., 2016) or via PSC to the endolymph based on reports (Guo et al., 2018; Tao et al., 2018). Briefly, after anesthesia, a retroauricular incision was made to approach the temporal bone. For intra-tympanic delivery onto the RWM, the otic bulla was identified ventral to the facial nerve and a shallow hole was made in the thin part of the otic bulla with a 30-G needle and enlarged with forceps to a diameter of 2 mm in order to visualize the round window. A customized sterile micro medical tube was inserted into the hole just above the round window niche (RWN) to slowly deliver 10 μL (0.6 μg) of siAMPKα or siControl. For PSC delivery, the sternocleidomastoid muscle was separated to expose the PSC. A small hole was made with the tip of a 30-G needle on the PSC and left open for about 1 min (there was no obvious endolymph leakage). Then the tip of the polyimide tube was inserted into the PSC toward the ampulla, and 1 μL of 0.6 μg siRNA was slowly microinjected for about 10 minutes. After the siRNA delivery, the hole was covered with surrounding muscle and glued with tissue adhesive. Finally, the skin incision was closed with tissue adhesive and the mouse was kept in the surgical position (less than 1 h) until it had woken up. Forty-eight hours after siRNA delivery, the animals were treated once with KM and furosemide.

### Auditory Brainstem Response and DPOAE Measurements

ABRs were measured in the left ears of anaesthetized mice before and two weeks after KM treatment. Mice were anesthetized with an intraperitoneal injection of a mixture of ketamine (100 mg/kg) and xylazine (10 mg/kg), and then placed in a sound-isolated and electrically shielded booth (Acoustic Systems). Body temperature was monitored and maintained near 37° C. with a heating pad. Acoustic stimuli were delivered monaurally to a Beyer earphone attached to a customized plastic speculum inserted into the ear canal. Subdermal electrodes were inserted at the vertex of the skull (active), mastoid region under the left ear (reference), and mastoid region under the right ear (ground). ABRs were measured at 8, 16, and 32 kHz. Tucker Davis Technology (TDT) System III hardware and SigGen/Biosig software were used to present the stimuli (15 ms duration tone bursts with 1 ms rise-fall time) and record the response. Up to 1024 responses were averaged for each stimulus level. ABR wave I was used to determine ABR thresholds for each frequency. Thresholds were determined for each frequency by reducing the intensity in 10-dB increments and then in 5-dB steps near threshold until no organized responses were detected. Thresholds were estimated between the lowest stimulus level where a response was observed and the highest level without response. All ABR measurements were conducted by the same experimenter. The ABR values were assigned by an expert who was blinded to the treatment conditions. Distortion product optoacoustic emissions (DPOAE) testing was performed in mice under anesthesia described above for ABRs. The emissions were measured using TDT RZ6 System and SigGen software. The acoustic assembly containing an ER-10B+ microphone connected to two transducers was tightly apposed into the ear canal. Primary tones were presented at fixed intensity levels of L1=65 dB SPL and L2=55 dB SPL with f2/f1 ratio of 1.2. Physiological results of each mouse were analyzed for individual frequencies, and then averaged for each of these frequencies from 4 kHz to 45 kHz.

### Immunolabeling of Cochlear Surface Preparations

The temporal bones were removed and perfused locally with a solution of 4% paraformaldehyde in PBS, pH 7.4, and kept in this fixative overnight at 4° C. For immunolabeling of inner hair cell synapses, the cochleae were perfused locally with a solution of 4% paraformaldehyde in PBS, pH 7.4, and kept in this fixative for 1.5 h at room temperature. Between every step, the cochlear samples were washed at least three time with PBS for 5-10 min each wash. After decalcification with 4% sodium EDTA solution (adjusted with HCl to pH 7.4) for 3 days at 4° C., the cochleae were micro-dissected into three turns (apex, middle, and base) and adhered to with cell-Tak (BD Biosciences, cat #354240). The specimens were first permeabilized in 3% Triton X-100 solution and then blocked with 10% normal goat serum each for 30 min at room temperature, followed by incubation with a monoclonal primary antibody of p-AMPKα at 1:100 (catalog #2535; Cell Signaling Technology) at 4° C. for 48 h. The specimens were then incubated with the Alexa Flour 594-conjugated secondary antibody at a concentration of 1:200 at 4° C. overnight and followed with incubation with Alexa Flour 488-phalloidin for 1 h at room temperature in darkness. Control incubations were routinely processed without primary antibody treatments.

All immunolabeling samples on round coverslips, after at least three final washes with PBS, were mounted using Fluoro-gel with Tris buffer (Electron Microscopy Sciences, #17985-10) with another round coverslip to sandwiches the samples and placed on slides. Finally, samples were sealed with nail polish. Immunolabeled images were taken with a 63×-magnification lens under identical Z-stack conditions using a Zeiss LSM 880 microscope.

Unless otherwise specified, all chemicals and reagents used were purchased from Sigma Aldrich.

Semi-Quantification of the Immunofluorescence Signals from Surface Preparations

Immunohistochemistry is well accepted as a semi-quantitative methodology when used with careful consideration of the utility and semi-quantitative nature of these assays (Taylor & Levenson, 2006; Walker, 2006). (Taylor & Levenson, 2006; Walker, 2006). The specificity of antibodies must be first detected by Western blot analysis. Antibodies showing only a single band with the correct molecular weight were used for immunolabeling on surface preparations and quantification of signaling in OHCs. The regions of interest were outlined within individual OHCs based on the counterstaining. The grayscale value was determined in only the hair cells to quantify the change in fluorescence intensity. This procedure provided quantitative measurements that are not confounded by protein expression in other cell types of the cochlea.

Immunolabeling for p-AMPKα was quantified from original confocal images, each taken with a 63×-magnification lens under identical conditions and equal parameter settings for laser gains and PMT gains, using ImageJ software (National Institutes of Health, Bethesda, Md.). The cochleae from the different groups were fixed and stained simultaneously with identical solutions and processed in parallel. All of the surface preparations were counter-stained with Alexa Fluor 488 phalloidin (green) to label hair cell structure in order to identify the comparable parts of the hair cells in confocal images. The regions of interest of individual OHCs were outlined with the circle tool based on phalloidin staining. The immunolabeling for the target proteins were measured in the upper-basal region (corresponding to sensitivity to 22-32 kHz) of cochlear surface preparations in 0.12-mm segments, each containing about 60 OHCs. The intensity of the background was subtracted and average grayscale intensity per cell was then calculated. For each repetition, the relative grayscale value was determined by normalizing the ratio to control.

### Immunohistochemistry of Cochlear Epithelia for Hair Cell Counts

Following decalcification and cochlear micro-dissection, the specimens were performed immunolabeling procedure as described above. The specimens were washed with PBS three times between each step and incubated with the primary antibody of rabbit polyclonal anti-myosin VII at a 1:200 dilution overnight at 4° C., and then incubated with biotinylated goat anti-rabbit antibody at a 1:1000 dilution overnight at 4° C. Subsequently, the specimens were incubated in ABC solution (Vector Laboratories #PK-4001) overnight and stained in DAB for 1-5 min, as necessary for sufficient staining intensity, followed by washing to stop the DAB reaction. Finally, the specimens were mounted on slides with mounting media. Images were taken with a Zeiss AxioCam MRc5 camera with Axioplan 2 imaging software under a Zeiss microscope.

Images from the apex through the base of the DAB-stained preparations were captured using a 20× lens on the Zeiss microscope. The lengths of the cochlear epithelia were measured and recorded in millimeters. OHCs were counted from the apex to the base along the entire length of the mouse cochlear epithelium. The percentage of hair cell loss in each 0.5-mm length of epithelium was plotted as a function of the cochlear length as a cytocochleogram (Chen et al., 2012; Zheng et al., 2014).

Mapping of frequencies as a function of distance was calculated with the equation [d (%)=156.5−82.5×log(f)] from Miller's paper (Muller et al., 2005) and agreed with the literature (Viberg & Canlon, 2004).

### Statistical Analyses

Data were analyzed using IBM SPSS Statistics Premium V21 and GraphPad software for Windows. Biological sample sizes were determined based on the variability of measurements and the magnitude of the differences between groups, as well as experience from our previous studies, with stringent assessments of difference. Data of OHC loss along the length of the cochlear spiral were analyzed with repeated measures one-way analysis of variance (ANOVA) with Tukey's multiple comparisons using IBM SPSS Statistics. The remaining analysis was done using GraphPad. Differences with multiple comparisons were evaluated by one-way ANOVA with multiple comparisons. Differences for single-pair comparisons were analyzed using two-tailed unpaired Student's t-tests. Data for relative ratios of single-pair comparisons were analyzed with one-sample t-tests. A p-value <0.05 was considered statistically significant. Data are presented as means±SD or SEM based on the sample size and variability within groups. Sample sizes are indicated for each figure.

### Example 13—Silencing siAMPKα1 does not Influence Outer Hair Cell Mechanotransduction Channels

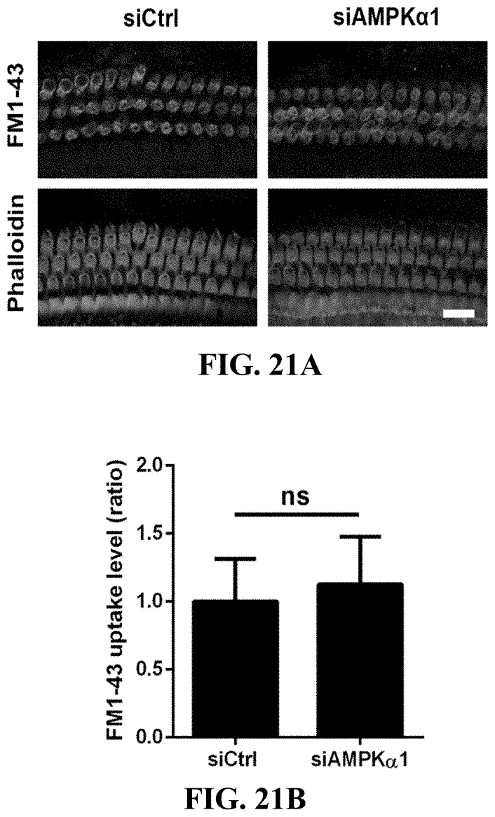

Since aminoglycoside antibiotics are known to enter outer hair cells (OHCs) mainly through mechanotransduction channels (MTCs), to address if protective effects of pretreatment with siAMPKα1 is due to blockade of kanamycin and/or furosemide entering OHCs, we assessed OHC MTCs using FM1-43FX, based on prior literature (Meyers et al., 2003; Taylor et al., 2008; Nakanishi et al., 2018). siAMPKα was delivered through the posterior semicircular canal (PSC) 48 h before application of FM1-43 to measure MTC function in OHCs. These results showed that pretreatment with siAMPKα1 via the PSC does not change FM1-43 uptake into OHCs compared with that of the siControl mice. FIG. 21A shows Images of green fluorescence reveal uptake of FM1-43 in OHCs. Scale bar=10 μm. FIG. 21B shows a semi-quantitative analysis of the grayscales of green fluorescence intensity shows no significant difference between siControl and siAMPKα groups. Data are presented as means+SD; ns: not significant. These results demonstrate that pretreatment with siAMPKα1 does not influence OHC mechanotransduction channel function and indicate that the protective effect against kanamycin-furosemide-induced loss of OHCs by siAMPKα1 is not due to inhibition of KM uptake.

### REFERENCES FOR EXAMPLES 1-6

Chen, F. Q., Zheng, H. W., Hill, K. & Sha, S. H. (2012) Traumatic Noise Activates Rho-Family GTPases through Transient Cellular Energy Depletion. J Neurosci, 32, 12421-12430.
Forge, A. & Schacht, J. (2000) Aminoglycoside antibiotics. Audiol Neurootol, 5, 3-22.
Guo, J. Y., He, L., Qu, T. F., Liu, Y. Y., Liu, K., Wang, G. P. & Gong, S. S. (2018) Canalostomy As a Surgical Approach to Local Drug Delivery into the Inner Ears of Adult and Neonatal Mice. J Vis Exp.
Hill, K., Yuan, H., Wang, X. & Sha, S. H. (2016) Noise-Induced Loss of Hair Cells and Cochlear Synaptopathy Are Mediated by the Activation of AMPK. J Neurosci, 36, 7497-7510.
Muller, M., von Hunerbein, K., Hoidis, S. & Smolders, J. W. (2005) A physiological place-frequency map of the cochlea in the CBA/J mouse. Hear Res, 202, 63-73.
Meyers et al., (2003) Lighting up the senses: FM1-43 loading of sensory cells through nonselective ion channels, J. Neurosci, 23, 4054-65.
Nakanishi et al., (2018) Tmc2 expression partially restores auditory function in a mouse model of DFNB7/B11 deafness caused by loss of Tmc1 function. Sci Rep, 8, 12125.
Oishi, N., Chen, F. Q., Zheng, H. W. & Sha, S. H. (2013) Intra-tympanic delivery of short interfering RNA into the adult mouse cochlea. Hear Res, 296, 36-41.
Ruhl, D., Du, T. T., Wagner, E. L., Choi, J. H., Li, S., Reed, R., Kim, K., Freeman, M., Hashisaki, G., Lukens, J. R. & Shin, J. B. (2019) Necroptosis and Apoptosis Contribute to Cisplatin and Aminoglycoside Ototoxicity. J Neurosci, 39, 2951-2964.
Sha, S. H. & Schacht, J. (2017) Emerging therapeutic interventions against noise-induced hearing loss. Expert Opin Investig Drugs, 26, 85-96.
Tao, Y., Huang, M., Shu, Y., Ruprecht, A., Wang, H., Tang, Y., Vandenberghe, L. H., Wang, Q., Gao, G., Kong, W. J. & Chen, Z. Y. (2018) Delivery of Adeno-Associated Virus Vectors in Adult Mammalian Inner-Ear Cell Subtypes Without Auditory Dysfunction. Hum Gene Ther, 29, 492-506.
Taylor, C. R. & Levenson, R. M. (2006) Quantification of immunohistochemistry—issues concerning methods, utility and semiquantitative assessment II. Histopathology, 49, 411-424.
Taylor, R. R., Nevill, G. & Forge, A. (2008) Rapid hair cell loss: a mouse model for cochlear lesions. J Assoc Res Otolaryngol, 9, 44-64.
Viberg, A. & Canlon, B. (2004) The guide to plotting a cochleogram. Hear Res, 197, 1-10.
Walker, R. A. (2006) Quantification of immunohistochemistry—issues concerning methods, utility and semiquantitative assessment I. Histopathology, 49, 406-410.
Wan, G., Gomez-Casati, M. E., Gigliello, A. R., Liberman, M. C. & Corfas, G. (2014) Neurotrophin-3 regulates ribbon synapse density in the cochlea and induces synapse regeneration after acoustic trauma. eLife, 3.
Yuan, H., Wang, X., Hill, K., Chen, J., Lemasters, J., Yang, S. M. & Sha, S. H. (2015) Autophagy attenuates noise-induced hearing loss by reducing oxidative stress. Antioxid Redox Signal, 22, 1308-1324.

### REFERENCES FOR EXAMPLES 7-13

Chen, F. Q., Zheng, H. W., Hill, K. & Sha, S. H. (2012) Traumatic Noise Activates Rho-Family GTPases through Transient Cellular Energy Depletion. J Neurosci, 32, 12421-12430.
Forge, A. & Schacht, J. (2000) Aminoglycoside antibiotics. Audiol Neurootol, 5, 3-22.
Guo, J. Y., He, L., Qu, T. F., Liu, Y. Y., Liu, K., Wang, G. P. & Gong, S. S. (2018) Canalostomy As a Surgical Approach to Local Drug Delivery into the Inner Ears of Adult and Neonatal Mice. J Vis Exp.
Hill, K., Yuan, H., Wang, X. & Sha, S. H. (2016) Noise-Induced Loss of Hair Cells and Cochlear Synaptopathy Are Mediated by the Activation of AMPK. J Neurosci, 36, 7497-7510.
Muller, M., von Hunerbein, K., Hoidis, S. & Smolders, J. W. (2005) A physiological place-frequency map of the cochlea in the CBA/J mouse. Hear Res, 202, 63-73.
Meyers et al., (2003) Lighting up the senses: FM1-43 loading of sensory cells through nonselective ion channels, J. Neurosci, 23, 4054-65.
Nakanishi et al., (2018) Tmc2 expression partially restores auditory function in a mouse model of DFNB7/B11 deafness caused by loss of Tmc1 function. Sci Rep, 8, 12125.
Oishi, N., Chen, F. Q., Zheng, H. W. & Sha, S. H. (2013) Intra-tympanic delivery of short interfering RNA into the adult mouse cochlea. Hear Res, 296, 36-41.
Ruhl, D., Du, T. T., Wagner, E. L., Choi, J. H., Li, S., Reed, R., Kim, K., Freeman, M., Hashisaki, G., Lukens, J. R. & Shin, J. B. (2019) Necroptosis and Apoptosis Contribute to Cisplatin and Aminoglycoside Ototoxicity. J Neurosci, 39, 2951-2964.
Sha, S. H. & Schacht, J. (2017) Emerging therapeutic interventions against noise-induced hearing loss. Expert Opin. Investig Drugs, 26, 85-96.
Tao, Y., Huang, M., Shu, Y., Ruprecht, A., Wang, H., Tang, Y., Vandenberghe, L. H., Wang, Q., Gao, G., Kong, W. J. & Chen, Z. Y. (2018) Delivery of Adeno-Associated Virus Vectors in Adult Mammalian Inner-Ear Cell Subtypes Without Auditory Dysfunction. Hum Gene Ther, 29, 492-506.
Taylor, C. R. & Levenson, R. M. (2006) Quantification of immunohistochemistry—issues concerning methods utility and semiquantitative assessment II. Histopathology, 49, 411-424.
Taylor, R. R., Nevill, G. & Forge, A. (2008) Rapid hair cell loss: a mouse model for cochlear lesions. J Assoc Res. Otolaryngol, 9, 44-64.
Viberg, A. & Canlon, B. (2004) The guide to plotting a cochleogram. Hear Res, 197, 1-10.
Walker, R. A. (2006) Quantification of immunohistochemistry—issues concerning methods, utility and semiquantitative assessment I. Histopathology, 49, 406-410.
Wu, W. J., Sha, S. H., McLaren, J. D., Kawamoto, K., Raphael, Y. & Schacht, J. (2001) Aminoglycoside ototoxicity in adult CBA, C57BL and BALB mice and the Sprague-Dawley rat. Hear Res, 158, 165-178.
Zheng, H. W., Chen, J. & Sha, S. H. (2014) Receptor-interacting protein kinases modulate noise-induced sensory hair cell death. Cell Death Dis, 5, e1262.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A method of treating or preventing an acquired (i) hearing loss, (ii) ototoxicity, (iii) outer hair cell damage or death, (iv) loss of an outer hair cell function, or a combination thereof comprising:
   a. administering an amount of an AMPK alpha targeting RNAi molecule to the subject in need thereof,
   b. administering an amount of a CaMKKβ targeting RNAi molecule to the subject in need thereof, or
   c. a combination thereof.
2. The method of aspect 1, wherein the AMPK alpha targeting RNAi molecule is capable of specifically binding a target sequence in an AMPK alpha gene product, the CaMKKβ targeting RNAi molecule is capable of specifically binding a CaMKKβ gene product, or both.
3. The method of aspect 2, wherein the target sequence in the CaMKKβ gene product
   a. is 50-100 percent identical to one or more sequences selected from the group consisting of SEQ ID NOs: 1-24; or
   b. is 50-100 percent identical to one or more sequences that are complementary to one or more sequences selected from the group consisting of SEQ ID NOs: 1-24.
4. The method of any one of aspects 2-3, wherein the target sequence in the AMPK alpha gene product
   a. is 50-100 percent identical to one or more sequences selected from the group consisting of SEQ ID NOs: 25-72; or
   b. is 50-100 percent identical to one or more sequences that are complementary to one or more sequences selected from the group consisting of SEQ ID NOs: 25-72.
5. The method of any one of aspects 2-4, wherein the AMPK alpha gene product is an mRNA, wherein the CaMKKβ gene product is an mRNA, or both.
6. The method of any one of aspects 2-5, wherein the AMPK alpha gene product is an AMPK alpha isoform 1 encoding polynucleotide or an AMPK alpha isoform 2 encoding polynucleotide.
7. The method of any one of aspects 1-6, wherein the AMPK alpha targeting RNAi molecule is a polynucleotide having a sequence that
   a. is 50-100 percent identical to a sequence selected from the group consisting of SEQ ID NOs: 25-72; or
   b. is 50-100 percent identical to a sequence that is complementary to a sequence selected from the group consisting of SEQ ID NOs: 25-72.
8. The method of any one of aspects 1-7, wherein administering the AMPK alpha targeting RNAi molecule, the CaMKKβ targeting RNAi molecule, or both occurs
   a. prior to exposure of the subject to an ototoxic agent;
   b. after exposure of the subject to an ototoxic agent; or
   c. both.
9. The method of aspect 8, wherein the ototoxic agent is a selected from the group consisting of: an antibiotic, a chemotherapeutic agent, a diuretic, a non-steroidal anti-inflammatory agent, an anti-malarial agent, an industrial solvent, an anticonvulsant agent, a psychopharmacologic agent, a cardiac or blood pressure therapeutic agent, an anti-allergy agent, a quinine-based agent, a glucocorticosteroid, an anesthetic, or a combination thereof.
10. The method of any one of aspects 8-9, wherein the ototoxic agent is an aminoglycoside antibiotic.
11. The method of any one of aspects 8-10, wherein the ototoxic agent is gentamicin, neomycin, kanamycin, amikacin, streptomycin, tobramycin, netilmicin, vancomycin, erythromycin, or a combination thereof.
12. The method of any one of aspects 8-11, wherein the ototoxic agent is a platinum-based chemotherapeutic agent.
13. The method of any one of aspects 8-12, wherein the ototoxic agent is cisplatin, carboplatin, oxaliplatin, nitrogen mustard, methotrexate, vincristine, dactinomycin, bleomycin, or any combination thereof.
14. The method of any one of aspects 9-13, wherein the diuretic is furosemide, bumetanide, ethacrynic acid, torsemide, chlor-thalidone, or any combination thereof.
15. The method of any one of aspects 9-14, wherein the non-steroidal anti-inflammatory agent is an acetic acid-based NSAID, a COX-2 inhibitor, a fenamate, an oxicam, a propionic acid, a salicylate, or a miscellaneous NSAID, or any combination thereof.
16. The method of any one of aspects 9-15, wherein the quinine-based agent is chloroquine phosphate, auinacrine hydrochloride, quinine sulfate, or any combination thereof.
17. The method of any one of aspects 9-16, wherein the anti-malarial agent is chloroquine and hydroxychloroquine.
18. The method of any one of aspects 9-17, wherein the cardiac or blood pressure therapeutic agent is celiprolol, flecaninide, lidocaine, metoprolol, procainamide, propranolol, or quinidine.
19. The method of any one of aspects 9-18, wherein the industrial solvent is cyclohexane, dichloromethane, hexane, indane, methyl-chloride, methyl-n-butyl-ketone, precholor-ethylene, styrene, tetrachlor-ethane, toluol, tricholorethylene, or any combination thereof.
20. The method of any one of aspects 9-19, wherein the anesthetic is bupivacaine, tetracaine, lidocaine, or a combination thereof.
21. The method of any one of aspects 9-20, wherein the glucocorticosteroid is prednisone, prednisolone, adrenocorticotrophic hormone, or any combination thereof.
22. The method of any one of aspects 9-21, wherein the psychopharmacologic agent is amitriptyline, a benzodiazepine, bupropion, carbamazepine, diclofensine, doxepin, desipramine, fluoxetine, imipramine, lithium, melitracen, molindone, paroxetine, phenelzine, protriptyline, trazodone, zimeldine, or any combination thereof.
23. The method of any one of aspects 9-22, the chemical ototoxic agent is alcohol, arsenic or arsenic-based compound, caffeine, lead, marijuana, nicotine, mercury, or auranofin, or any combination thereof.
24. The method of any one of aspects 9-13, wherein the AMPK alpha targeting RNAi molecule, the CaMKKβ targeting RNAi molecule, or both is/are administered directly to an ear of a subject.
25. The method of any one of aspects 9-24, wherein the AMPK alpha targeting RNAi molecule, the CaMKKβ targeting RNAi molecule, or both is/are administered via intra-tympanic delivery, intracochlear delivery, semicircular canal delivery, or a combination thereof.
26. The method of any one of aspects 9-25, wherein the AMPK alpha targeting RNAi molecule, the CaMKKβ targeting RNAi molecule, or both is/are administered via intra-tympanic delivery on the round window membrane.
27. The method of any one of aspects 9-16, wherein the AMPK alpha targeting RNAi molecule, the CaMKKβ targeting RNAi molecule, or both is/are administered via posterior semicircular canal delivery.
28. A method of reducing an amount of an AMPK alpha gene product, a CaMKKβ gene product, or both in a subject in need thereof, the method comprising:
   a. administering an amount of an AMPK alpha targeting RNAi molecule to the subject in need thereof,
   b. administering an amount of a CaMKKβ targeting RNAi molecule to the subject in need thereof, or
   c. a combination thereof.
29. The method of aspect 28, wherein the AMPK alpha targeting RNAi molecule is capable of specifically binding a target sequence in an AMPK alpha gene product, the CaMKKβ targeting RNAi molecule is capable of specifically binding a CaMKKβ gene product, or both.
30. The method of any one of aspects 28-29, wherein that target sequence in the CaMKKβ gene product
   a. is 50-100 percent identical to one or more sequences selected from the group consisting of SEQ ID NOs: 1-24; or
   b. is 50-100 percent identical to one or more sequences that are complementary to one or more sequences selected from the group consisting of SEQ ID NOs: 1-24.
31. The method of any one of aspects 29-30, wherein the target sequence in the AMPK alpha gene product
   a. is 50-100 percent identical to one or more sequences selected from the group consisting of SEQ ID NOs: 25-72; or
   b. is 50-100 percent identical to one or more sequences that are complementary to one or more sequences selected from the group consisting of SEQ ID NOs: 25-72.
32. The method of any one of aspects 29-31, wherein the AMPK alpha gene product is an mRNA, wherein the CaMKKβ gene product is an mRNA, or both.
33. The method of any one of aspects 29-32, wherein the AMPK alpha gene product is an AMPK alpha isoform 1 encoding polynucleotide or an AMPK alpha isoform 2 encoding polynucleotide.
34. The method of any one of aspects 28-32, wherein the AMPK alpha targeting RNAi molecule is a polynucleotide having a sequence that
   a. is 50-100 percent identical to a sequence selected from the group consisting of SEQ ID NOs: 25-72; or
   b. is 50-100 percent identical to a sequence that is complementary to a sequence selected from the group consisting of SEQ ID NOs: 25-72.
35. The method of any one of aspects 28-34, wherein the AMPK alpha targeting RNAi molecule, the CaMKKβ targeting RNAi molecule, or both is/are administered directly to an ear of a subject.
36. The method of any one of aspects 28-35, wherein the AMPK alpha targeting RNAi molecule, the CaMKKβ targeting RNAi molecule, or both is/are administered via intra-tympanic delivery, intracochlear delivery, semicircular canal delivery, or a combination thereof.
37. The method of any one of aspects 28-36, wherein the AMPK alpha targeting RNAi molecule, the CaMKKβ targeting RNAi molecule, or both is/are administered via intra-tympanic delivery on the round window membrane.
38. The method of any one of aspects 28-37, wherein the AMPK alpha targeting RNAi molecule, the CaMKKβ targeting RNAi molecule, or both is/are administered via posterior semicircular canal delivery.
39. A pharmaceutical formulation capable of treating or preventing an acquired (i) hearing loss, (ii) ototoxicity, (iii) outer hair cell damage or death, (iv) loss of an outer hair cell function, or a combination thereof comprising:
   a therapeutically effective amount of
      a. an AMPK alpha targeting RNAi molecule;
      b. a CaMKKβ targeting RNAi molecule; or
      c. both; and
   a pharmaceutically acceptable carrier.
40. The pharmaceutical formulation of aspect 39, wherein the AMPK alpha targeting RNAi molecule is capable of specifically binding a target sequence in an AMPK alpha gene product, the CaMKKβ targeting RNAi molecule is capable of specifically binding a CaMKKβ gene product, or both.
41. The pharmaceutical formulation of any one of aspects 39-40, wherein that target sequence in the CaMKKβ gene product
   a. is 50-100 percent identical to one or more sequences selected from the group consisting of SEQ ID NOs: 1-24; or
   b. is 50-100 percent identical to one or more sequences that are complementary to one or more sequences selected from the group consisting of SEQ ID NOs: 1-24.
42. The pharmaceutical formulation of any one of aspects 39-41, wherein the target sequence in the AMPK alpha gene product
   a. is 50-100 percent identical to one or more sequences selected from the group consisting of SEQ ID NOs: 25-72; or
   b. is 50-100 percent identical to one or more sequences that are complementary to one or more sequences selected from the group consisting of SEQ ID NOs: 25-72.
43. The pharmaceutical formulation of any one of aspects 39-40, wherein the AMPK alpha gene product is an mRNA, wherein the CaMKKβ gene product is an mRNA, or both.
44. The pharmaceutical formulation of any one of aspects 40-43, wherein the AMPK alpha gene product is an AMPK alpha isoform 1 encoding polynucleotide or an AMPK alpha isoform 2 encoding polynucleotide.
45. The pharmaceutical formulation of any one of aspects 39-44, wherein the AMPK alpha targeting RNAi molecule is a polynucleotide having a sequence that
   a. is 50-100 percent identical to a sequence selected from the group consisting of SEQ ID NOs: 25-72; or is
   b. 50-100 percent identical to a sequence that is complementary to a sequence selected from the group consisting of SEQ ID NOs: 25-72.
46. The pharmaceutical formulation of any one of aspects 39-45, wherein the AMPK alpha targeting RNAi molecule, the CaMKKβ targeting RNAi molecule, or both are contained in one or more vectors of a vector system.
47. The pharmaceutical formulation of any one of aspects 39-46, wherein AMPK alpha targeting RNAi molecule, the CaMKKβ targeting RNAi molecule, or both are effective to decrease or eliminate
   a. the amount of an AMPK alpha gene product;
   b. the amount of an CaMKKβ gene product;
   c. gene expression, protein expression, or both of AMPK alpha isoform 1, AMPK alpha isoform 2, or both;
   d. gene expression, protein expression, or both of CaMKKβ; or
   e. a combination thereof.
48. The pharmaceutical formulation of any one of aspects 29-47, wherein the pharmaceutical formulation is adapted for direct delivery to an ear of the subject.
49. The pharmaceutical formulation of any one of aspects 29-48, wherein the pharmaceutical formulation is adapted for intra-tympanic delivery, intracochlear delivery, semicircular canal delivery, or a combination thereof.
50. The pharmaceutical formulation of any one of aspects 29-49, wherein the pharmaceutical formulation is adapted for intra-tympanic delivery on the round window membrane, posterior semicircular canal delivery, or both.
51. A kit comprising:
   an AMPK alpha targeting RNAi molecule or a pharmaceutical formulation thereof, a CaMKKβ targeting RNAi molecule or a pharmaceutical formulation thereof; or both; and
   instructions fixed in a tangible medium of expression, wherein the instructions provide direction to administer the AMPK alpha targeting RNAi molecule or a pharmaceutical formulation thereof, the CaMKKβ targeting RNAi molecule or a pharmaceutical formulation thereof, or both to a subject in need thereof to
   a. treat or prevent an acquired
      i. hearing loss;
      ii. ototoxicity;
      iii. outer hair cell damage or death;
      iv loss of an outer hair cell; or
      v. a combination thereof;
   b. decrease or eliminate
      i. the amount of an AMPK alpha gene product;
      ii. the amount of an CaMKKβ gene product;
      iii gene expression, protein expression, or both of AMPK alpha isoform 1, AMPK alpha isoform 2, or both;
      iv. gene expression, protein expression, or both of CaMKKβ; or
      v. a combination thereof; or
   c. both
   in the subject.

What is claimed is:

1. A method of treating or preventing an acquired (i) hearing loss, (ii) ototoxicity, (iii) outer hair cell damage or death, (iv) loss of an outer hair cell function, or a combination thereof comprising:
a. administering an amount of an AMPK alpha targeting RNAi molecule to the subject in need thereof,
b. administering an amount of a CaMKKβ targeting RNAi molecule to the subject in need thereof, or
c. a combination thereof.

2. The method of claim 1, wherein the AMPK alpha targeting RNAi molecule is capable of specifically binding a target sequence in an AMPK alpha gene product, the CaMKKβ targeting RNAi molecule is capable of specifically binding a CaMKKβ gene product, or both.

3. The method of claim 2, wherein the target sequence in the CaMKKβ gene product
a. is 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

4. The method of claim 2, wherein the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

5. The method of claim 2, wherein the AMPK alpha gene product is an mRNA, wherein the CaMKKβ gene product is an mRNA, or both.

6. The method of claim 2, wherein the AMPK alpha gene product is an AMPK alpha isoform 1 encoding polynucleotide or an AMPK alpha isoform 2 encoding polynucleotide.

7. The method of claim 1, wherein the AMPK alpha targeting RNAi molecule is a polynucleotide having a sequence that
a. is 50-100 percent identical to a sequence selected from the group consisting of SEQ ID NOs: 25-72; or
b. is 50-100 percent identical to a sequence that is complementary to a sequence selected from the group consisting of SEQ ID NOs: 25-72.

8. The method of claim 1, wherein administering the AMPK alpha targeting RNAi molecule, the CaMKKβ targeting RNAi molecule, or both occurs
a. prior to exposure of the subject to an ototoxic agent;
b. after exposure of the subject to an ototoxic agent; or
c. both.

9. The method of claim 8, wherein the ototoxic agent is a selected from the group consisting of: an antibiotic, a chemotherapeutic agent, a diuretic, a non-steroidal anti-inflammatory agent, an anti-malarial agent, an industrial solvent, an anticonvulsant agent, a psychopharmacologic agent, a cardiac or blood pressure therapeutic agent, an anti-allergy agent, a quinine-based agent, a glucocorticosteroid, an anesthetic, or a combination thereof.

10. The method of claim 9, wherein the ototoxic agent is an aminoglycoside antibiotic.

11. The method of claim 9, wherein the ototoxic agent is gentamicin, neomycin, kanamycin, amikacin, streptomycin, tobramycin, netilmicin, vancomycin, erythromycin, or a combination thereof.

12. The method of claim 9, wherein the ototoxic agent is a platinum-based chemotherapeutic agent.

13. The method of claim 9, wherein the ototoxic agent is cisplatin, carboplatin, oxaliplatin, nitrogen mustard, methotrexate, vincristine, dactinomycin, bleomycin, or any combination thereof.

14. The method of claim 9, wherein the diuretic is furosemide, bumetanide, ethacrynic acid, torsemide, chlor-thalidone, or any combination thereof.

15. The method of claim 9, wherein the non-steroidal anti-inflammatory agent is an acetic acid-based NSAID, a COX-2 inhibitor, a fenamate, an oxicam, a propionic acid, a salicylate, or a miscellaneous NSAID, or any combination thereof.

16. The method of claim 9, wherein the quinine-based agent is chloroquine phosphate, auinacrine hydrochloride, quinine sulfate, or any combination thereof.

17. The method of claim 9, wherein the anti-malarial agent is chloroquine and hydroxychloroquine.

18. The method of claim 9, wherein the cardiac or blood pressure therapeutic agent is celiprolol, flecaninide, lidocaine, metoprolol, procainamide, propranolol, or quinidine.

19. The method of claim 9, wherein the industrial solvent is cyclohexane, dichloromethane, hexane, indane, methyl-chloride, methyl-n-butyl-ketone, precholor-ethylene, styrene, tetrachlor-ethane, toluol, tricholorethylene, or any combination thereof.

20. The method of claim 9, wherein the anesthetic is bupivacaine, tetracaine, lidocaine, or a combination thereof.

21. The method of claim 9, wherein the glucocorticosteroid is prednisone, prednisolone, adrenocorticotrophic hormone, or any combination thereof.

22. The method of claim 9, wherein the psychopharmacologic agent is amitriptyline, a benzodiazepine, bupropion, carbamazepine, diclofensine, doxepin, desipramine, fluoxetine, imipramine, lithium, melitracen, molindone, paroxetine, phenelzine, protriptyline, trazodone, zimeldine, or any combination thereof.

23. The method of claim 9, the chemical ototoxic agent is alcohol, arsenic or arsenic-based compound, caffeine, lead, marijuana, nicotine, mercury, or auranofin, or any combination thereof.

24. The method of claim 1, wherein the AMPK alpha targeting RNAi molecule, the CaMKKβ targeting RNAi molecule, or both is/are administered directly to an ear of a subject.

25. The method of claim 1, wherein the AMPK alpha targeting RNAi molecule, the CaMKKβ targeting RNAi molecule, or both is/are administered via intra-tympanic delivery, intracochlear delivery, semicircular canal delivery, or a combination thereof.

26. The method of claim 25, wherein the AMPK alpha targeting RNAi molecule, the CaMKKβ targeting RNAi molecule, or both is/are administered via intra-tympanic delivery on the round window membrane.

27. The method of claim 25, wherein the AMPK alpha targeting RNAi molecule, the CaMKKβ targeting RNAi molecule, or both is/are administered via posterior semicircular canal delivery.

28. A method of reducing an amount of an AMPK alpha gene product, a CaMKKβ gene product, or both in a subject in need thereof, the method comprising:
a. administering an amount of an AMPK alpha targeting RNAi molecule to the subject in need thereof,
b. administering an amount of a CaMKKβ targeting RNAi molecule to the subject in need thereof, or
c. a combination thereof.

29. The method of claim 28, wherein the AMPK alpha targeting RNAi molecule is capable of specifically binding a target sequence in an AMPK alpha gene product, the CaMKKβ targeting RNAi molecule is capable of specifically binding a CaMKKβ gene product, or both.

30. The method of claim 29, wherein that target sequence in the CaMKKβ gene product is
a. 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

31. The method of claim 29, wherein the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

32. The method of claim 29, wherein the AMPK alpha gene product is an mRNA, wherein the CaMKKβ gene product is an mRNA, or both.

33. The method of claim 29, wherein the AMPK alpha gene product is an AMPK alpha isoform 1 encoding polynucleotide or an AMPK alpha isoform 2 encoding polynucleotide.

34. The method of claim 28, wherein the AMPK alpha targeting RNAi molecule is a polynucleotide having a sequence that
a. is 50-100 percent identical to a sequence selected from the group consisting of SEQ ID NOs: 25-72; or
b. is 50-100 percent identical to a sequence that is complementary to a sequence selected from the group consisting of SEQ ID NOs: 25-72.

35. The method of claim 28, wherein the AMPK alpha targeting RNAi molecule, the CaMKKβ targeting RNAi molecule, or both is/are administered directly to an ear of a subject.

36. The method of claim 28, wherein the AMPK alpha targeting RNAi molecule, the CaMKKβ targeting RNAi molecule, or both is/are administered via intra-tympanic delivery, intracochlear delivery, semicircular canal delivery, or a combination thereof.

37. The method of claim 36, wherein the AMPK alpha targeting RNAi molecule, the CaMKKβ targeting RNAi molecule, or both is/are administered via intra-tympanic delivery on the round window membrane.

38. The method of claim 36, wherein the AMPK alpha targeting RNAi molecule, the CaMKKβ targeting RNAi molecule, or both is/are administered via posterior semicircular canal delivery.

39. A pharmaceutical formulation capable of treating or preventing an acquired (i) hearing loss, (ii) ototoxicity, (iii) outer hair cell damage or death, (iv) loss of an outer hair cell function, or a combination thereof comprising:
a therapeutically effective amount of
a. an AMPK alpha targeting RNAi molecule;
b. a CaMKKβ targeting RNAi molecule; or
c. both; and
a pharmaceutically acceptable carrier.

40. The pharmaceutical formulation of claim 39, wherein the AMPK alpha targeting RNAi molecule is capable of specifically binding a target sequence in an AMPK alpha gene product, the CaMKKβ targeting RNAi molecule is capable of specifically binding a CaMKKβ gene product, or both.

41. The pharmaceutical formulation of claim 40, wherein that target sequence in the CaMKKβ gene product
a. is 50-100 percent identical to one or more sequences selected from the group consisting of SEQ ID NOs: 1-24; or
b. is 50-100 percent identical to one or more sequences that are complementary to one or more sequences selected from the group consisting of SEQ ID NOs: 1-24.

42. The pharmaceutical formulation of claim 40, wherein the target sequence in the AMPK alpha gene product
a. is 50-100 percent identical to one or more sequences selected from the group consisting of SEQ ID NOs: 25-72; or
b. is 50-100 percent identical to one or more sequences that are complementary to one or more sequences selected from the group consisting of SEQ ID NOs: 25-72.

43. The pharmaceutical formulation of claim 40, wherein the AMPK alpha gene product is an mRNA, wherein the CaMKKβ gene product is an mRNA, or both.

44. The pharmaceutical formulation of claim 40, wherein the AMPK alpha gene product is an AMPK alpha isoform 1 encoding polynucleotide or an AMPK alpha isoform 2 encoding polynucleotide.

45. The pharmaceutical formulation of claim 39, wherein the AMPK alpha targeting RNAi molecule is a polynucleotide having a sequence that
a. is 50-100 percent identical to a sequence selected from the group consisting of SEQ ID NOs: 25-72; or is
b. 50-100 percent identical to a sequence that is complementary to a sequence selected from the group consisting of SEQ ID NOs: 25-72.

46. The pharmaceutical formulation of any one of claims 39-45, wherein the AMPK alpha targeting RNAi molecule, the CaMKKβ targeting RNAi molecule, or both are contained in one or more vectors of a vector system.

47. The pharmaceutical formulation of any one of claims 39-45, wherein AMPK alpha targeting RNAi molecule, the CaMKKβ targeting RNAi molecule, or both are effective to decrease or eliminate
a. the amount of an AMPK alpha gene product;
b. the amount of an CaMKKβ gene product;
c. gene expression, protein expression, or both of AMPK alpha isoform 1, AMPK alpha isoform 2, or both;
d. gene expression, protein expression, or both of CaMKKβ; or
e. a combination thereof.

48. The pharmaceutical formulation of claim 47, wherein the pharmaceutical formulation is adapted for direct delivery to an ear of the subject.

49. The pharmaceutical formulation of claim 48, wherein the pharmaceutical formulation is adapted for intra-tympanic delivery, intracochlear delivery, semicircular canal delivery, or a combination thereof.

50. The pharmaceutical formulation of claim 49, wherein the pharmaceutical formulation is adapted for intra-tympanic delivery on the round window membrane, posterior semicircular canal delivery, or both.

51. A kit comprising:
an AMPK alpha targeting RNAi molecule or a pharmaceutical formulation thereof, a CaMKKβ targeting RNAi molecule or a pharmaceutical formulation thereof; or both; and
instructions fixed in a tangible medium of expression, wherein the instructions provide direction to administer the AMPK alpha targeting RNAi molecule or a pharmaceutical formulation thereof, the CaMKKβ targeting RNAi molecule or a pharmaceutical formulation thereof, or both to a subject in need thereof to
a. treat or prevent an acquired
i. hearing loss;
ii. ototoxicity;
iii. outer hair cell damage or death;
iv loss of an outer hair cell; or
v. a combination thereof;
b. decrease or eliminate
i. the amount of an AMPK alpha gene product;
ii. the amount of an CaMKKβ gene product;
iii gene expression, protein expression, or both of AMPK alpha isoform 1, AMPK alpha isoform 2, or both;
iv. gene expression, protein expression, or both of CaMKKβ; or
v. a combination thereof; or
c. both
in the subject.
